(19) United States
(12) Patent Application Publication
(10) Pub. No.: US 2011/0319283 A1
(43) Pub. Date: Dec. 29, 2011

(54) OLIGONUCLEOTIDES CAPABLE OF DISCRIMINATING BETWEEN NUCLEIC ACID SEQUENCES THAT COMPRISE A CONSERVED SEQUENCE

(75) Inventor: Andrew Thompson, Cambridgeshire (GB)

(73) Assignee: Trillion Genomics Limited, Cambridge, Cambridgeshire (GB)

(21) Appl. No.: 13/130,946

(22) PCT Filed: Nov. 24, 2009
(86) PCT No.: PCT/GB09/02748

§ 371 Date: Sep. 15, 2011

(30) Foreign Application Priority Data
Nov. 24, 2008 (GB) 0821457.9

(51) Int. Cl. C40B 30/04; C12Q 1/68; C07H 21/00; C40B 40/06; B82Y 15/00

(52) U.S. Cl. 506/9; 536/24.3; 506/16; 435/6.11; 977/773; 977/920

(57) ABSTRACT

The invention provides inter alia an oligonucleotide (or “probe”) able to discriminate between a nucleic acid target and a nucleic acid variant thereof (for example, mRNA splice variants, or chimeric gene and corresponding parent gene mRNAs) in which the target and variant that share at least one domain of conserved or identical sequence. The oligonucleotide in one aspect has a first portion and a second portion flanking a portion junction, wherein the first portion comprises at least a first discontinuity relative to the first domain of a target sequence and the second portion comprises at least a second discontinuity relative to a second domain of a target sequence, each discontinuity comprising or consisting of a sequence mismatch and/or a non-nucleotide spacer.

The present invention relates to the field of molecular biology and in particular to oligonucleotides for use in nucleic acid hybridisation, and to methods and reagents for distinguishing between nucleic acid sequences that comprise a conserved sequence.

The discovery of the complementary double-stranded helix structure of DNA and the development of methods to chemically synthesize oligonucleotides have made it possible to develop techniques for detecting a target nucleic acid sequence by hybridisation of a labelled synthetic oligonucleotide probe sequence complementary to at least a part of the sequence of the target nucleic acid.

The reliability of the hybridisation process depends inter alia upon specific recognition of the target sequence by the probe sequence. When a probe strand binds to a target strand having similar but not identical complementary sequence, the affinity of the two strands for one another is reduced but cross-hybridisation is still possible. If two targets with similar sequences are present in a biological sample, it may be difficult to discriminate between them where both targets bind to the same probe.

This is a particular problem in gene expression profiling, where the objective is to determine the relative or absolute quantities of different species of messenger RNA (mRNA) in a sample. In a process called splicing (or cis-splicing), non-coding introns or other sequences are removed from pre-mRNA (also known as heterogeneous nuclear RNA [hnRNA] or immature RNA), and the remaining exons which usually encode for polypeptides are joined together to produce mature mRNA. Another splicing process called trans-splicing involves ligation of two or more exons from different genes. Differential splicing by extending or skipping of one or more exons, or retaining of one or more introns, creates alternative mRNA splice variants of the same gene. Many genes which are subject to splicing have a modular structure in which domains or “blocks” of a gene sequence can be recombined in different ways to give alternative mRNA splice variants. The presence of such conserved or shared domains makes it difficult to distinguish different splice variants in hybridisation-dependent gene expression profiling assays.

Additionally, chromosomal translocations or deletions may result in two “parent” genes being fused to form a “chimeric” gene (also referred to as a “gene fusion”) and a mutant chimeric gene product may be expressed. The chimeric gene comprises part of the sequence of one parent gene and part of the sequence of another parent gene. Chimeric genes are found in certain cancers such as leukemia, where the presence of particular translocations or deletions is associated with the severity of the disease. One characteristic fusion found in leukemia is a BCR-ABL gene fusion (“B2A2”; see Bohlander, 2000, Cytogenet Cell Genet. 91(1-4):52-6). The best mode of treatment by targeting and killing cancer cells which express chimeric genes may be determined following identification of the presence of these fused sequences. It would be useful to be able to reliably detect a chimeric gene (or its mRNA) in a simple assay.

To enable comprehensive analysis of gene expression in the human genome, it would in theory be useful to functionally resolve each mRNA species in a sample with a specific oligonucleotide probe. However, to achieve this it would be necessary to generate specific probes for every alternative splice variant. It is estimated that 40-60% of the genes in the human genome have at least one splice variant (Modrek & Lee, 2002, Nat Genet. 30: 13-19). Designing probes which distinguish splice variants is a challenging task.

Consider for example a gene comprising the exons A, B and C, with two splice variants “A-C” and “A-B-C”. Detecting the presence of both variants can be achieved with probes for exon A or C. The presence of the second variant could be determined using a probe for exon B, while the presence of the first variant could be identified by the presence of A or C but not B. However, if both variants are present in a sample, the presence of the variant “A-C” could not be ascertained with exon-specific probes for exon A and exon C only, as these exons are shared by the variants.

It has been proposed that boundary-spanning oligonucleotide probes complementary to sequences flanking juxtaposed domain sequences can be used to detect splice variants or gene fusion mRNAs (Kane et al., 2000, Nucleic Acids Res. 28: 4552-4557). The experimental results for that approach using 50-mer oligonucleotides showed significant cross-hybridisation of different splice variants to the boundary-spanning probes. This may not be unexpected since splice variants that share one exon spanned by a boundary-spanning probe can hybridise to part of the sequence of the probe. Although cross-hybridisation may in theory be less of a problem for shorter oligonucleotides as the melting temperature (Tₘ) of a shorter sequence will tend to be lower than for a longer boundary-spanning probe, the sensitivity of shorter probes is considerably lower than for longer probes (Religion et al., 2002, Nucleic Acids Res. 30: e51). As used herein, Tₘ is defined as the temperature at which 50% of an equimolar solution of an oligonucleotide and its perfect complement are hybridised in a duplex.

The problem of detecting a splice variant or a chimeric gene mRNA is compounded when attempting to perform in situ hybridisation, as corresponding chromosomal gene sequence(s) and pre-mRNA may additionally be present. Also, amplification of the target may be difficult or impossible, so any probe used should be highly sensitive to detect. Furthermore, generally speaking it is necessary to have high affinity probes for in situ hybridisation to assist binding in the presence of extensive secondary structure and auxiliary proteins bound to RNA and also to ensure that the probe remains bound to the target after extensive wash steps to remove unbound probe. The use of short probes may thus not be a suitable option for improving detection of alternative splice variants or chimeric genes in situ.

Recently, micro-fabrication techniques have enabled the development of oligonucleotide arrays that allows the simultaneous detection of multiple sequences present in a sample. Typically, nucleic acids in a sample are labelled with a fluorescent dye and the labelled material is hybridised to oligonucleotide probes on an array. The array may be designed such that individual probes are present at discrete locations on the array, allowing determination of the presence of a particular target sequence by measuring the amount of fluorescence that is emitted from each probe location on the array. Various techniques have been developed for labelling of large populations of nucleic acids in a single reaction, particularly techniques for labelling populations of poly-adenylated messenger RNA molecules. This combination of mRNA labelling techniques and the availability of oligonucleotide array technology has enabled researchers to perform comprehensive gene expression profiling studies on numerous biological systems. However, in the simplest and most convenient formats, this technology is dependent on being able to reliably distinguish different target sequences based on the specificity of hybridisation of oligonucleotide probes to labelled target nucleic acids.

Antisense oligonucleotides have been proposed as a method for inhibiting the expression of particular genes (Crooke, 1998, Antisense Nucleic Acid Drug Dev. 8(2): 133-4) or for changing patterns of alternative splicing (Wilton & Fletcher, 2005, Gene Ther. 5(5): 467-83). For the purposes of detecting splice variants or chimeric gene mRNAs, highly specific antisense reagents are required.

The present invention provides inter alia an oligonucleotide (also referred to herein as a “probe”) able to discriminate between a nucleic acid target and a nucleic acid variant thereof (for example, mRNA splice variants, or chimeric gene and corresponding parent gene mRNAs) in which the target and variant that share at least one domain of conserved or identical sequence.

The present invention also provides an array of two or more oligonucleotides able to discriminate between a nucleic acid target and a variant thereof that shares at least one domain of conserved or identical sequence.

Also provided according to the invention is an antisense oligonucleotide able to specifically hybridise with a target sequence comprising a domain structure, for example, a target sequence which shares at least one domain of conserved or identical sequence with a variant of the target sequence.

The oligonucleotides of the invention are particularly suitable for in situ hybridisation, in vivo applications like antisense therapy, and also for other applications like microarray detection of alternative splice variants or chromosomal translocations in cancer.

According to the present invention, there is provided in one aspect an oligonucleotide (such as an oligonucleotide probe, also referred to herein as a “probe”) which is hybridisable with greater affinity to a target nucleic acid than to a nucleic acid variant of the target nucleic acid, the target nucleic acid comprising a first domain and a second domain flanking a domain junction, the first domain having a sequence which is conserved with a first sequence in the nucleic acid variant, wherein the oligonucleotide has a first portion and a second portion flanking a portion junction, the first portion being complementary in part to the first domain and the second portion being complementary in part to the second domain, but wherein the first portion comprises at least a first discontinuity relative to the first domain and the second portion comprises at least a second discontinuity relative to the second domain, each discontinuity comprising or consisting of a sequence mismatch and/or a non-nucleotide spacer.

As elaborated below, the oligonucleotides utilise discontinuities in their sequence to enhance specificity (also referred to herein as “discrimination”). Oligonucleotide probes having discontinuities are known in the prior art, but not with the structure and uses of the presently described oligonucleotides.

As illustrated in specific examples, the oligonucleotide of the invention comprising at least two discontinuities will typically bind to a target nucleic acid (also referred to herein as a “target” or “target sequence”) with a lower affinity and consequently with a lower Tₘ than a corresponding oligonucleotide that is fully complementary to the same target. Thus, the oligonucleotide of the present invention will be slightly less sensitive than a corresponding fully complementary oligonucleotide of the same length. However, the difference between the Tₘ of a duplex formed by the oligonucleotide of the invention and its target compared to the Tₘ of the duplex formed by the oligonucleotide of the invention and a nucleic acid variant comprising only one domain of conserved or identical sequence will be larger than that for a corresponding fully complementary oligonucleotide which lacks a discontinuity. This feature of the oligonucleotide of the invention allows enhanced detection of the target vs variant nucleic acids.

Introduction of an “artificial mismatch” into an oligonucleotide probe sequence has been shown to enhance the specificity of the recognition of single nucleotide polymorphisms (“SNPs”; see Guo et al., 1997, Nat Biotechnol. 15(4): 331-5; U.S. Pat. No. 5,780,233). Guo et al. describe probes that can act as polymerase chain reaction (PCR) primers in SNP discrimination reactions and as simple hybridisation probes. However, Guo et al. do not teach that artificial mismatches in oligonucleotide probes could be used to enhance discrimination of sequences with conserved or identical domains.

It has also been shown that “tethered oligonucleotide probes” comprising two sequences linked by a single non-nucleic acid linker can have enhanced specificity for mRNA targets with extensive secondary structure, compared to single sequences of the same length (Cload & Schepartz, 1994, J. Am. Chem. Soc. 116: 437-442). However, again such tethered probes do not have the structure or uses of the presently described oligonucleotides.

The oligonucleotide of the present invention may have first and second portions of substantially equal length, for example lengths which do not differ by more than 0-10% or 0-5% in terms of numbers of monomers (such as nucleotides and discontinuities). The first and second portions may be structurally bilaterally symmetrical about the portion junction. Thus the first and second portions may have the same number of monomers on each side of the portion junction, although the sequence of the first and second portions will usually not be the same.

The first and second portions of the oligonucleotide may have substantially the same Tₘ. By “substantially the same” is meant that the Tₘ of the first portion is identical with, or up to 10% different from, for example up to 9%, 8%, 7%, 6%, 5%, 4%, 3%, 2% or 1% different from, the Tₘ of the second portion. As exemplified below, it has been found that discrimination of the oligonucleotide between the target nucleic acid and its target nucleic acid variant(s) may be enhanced by using such an oligonucleotide.

The Tₘ of the first and second portions may be calculated theoretically, for example using the Nearest Neighbour method (see Breslauer et al., 1986, PNAS USA 83(11): 3746-50; SantaLucia et al., 1996, Biochemistry 35(11): 3555-62; Xia et al., 1998, Biochemistry 37(42):14719-35; Kierzek et al., 2006, Nucleic Acids Res. 34(13): 3609-14; McTigue et al., 2004, Biochemistry. 43(18): 5388-405).

Where the first and second portions have substantially the same Tₘ, these portions will typically, but not necessarily, be of unequal length.

Each of the first and second discontinuities may be positioned adjacent nucleotide 2 to 20 (i.e. adjacent nucleotide 2, 3, 4, 5, 6, 7, 8, 9, 10, 11, 12, 13, 14, 15, 16, 17, 18, 19 or 20), for example adjacent nucleotide 3 or nucleotide 4 or adjacent nucleotide 8, 9, 10, 11 or 12, of the oligonucleotide, relative to the portion junction.

Each discontinuity may be of a length equivalent to 1 to 5 nucleotides, for example 1, 2, 3, 4, or 5 nucleotides, such as 1 or 2 nucleotides.

Each sequence mismatch may comprise a natural nucleotide which is non-complementary to a base at a corresponding position in the target nucleic acid. Alternatively, the sequence mismatch may comprise an artificial mismatch. The oligonucleotide may comprise a mixture of one or more natural nucleotide mismatches and one or more artificial mismatches. The artificial mismatch may comprise a universal base analogue or an abasic mismatch.

Each discontinuity may be a non-nucleotide spacer, for example polyethylene glycol, a phosphoramidite spacer such as a C3 phosphoramidite spacer, or an amino acid.

The oligonucleotide may comprise a natural nucleotide or a nucleotide analogue, for example a 2-O-methyl analogue, a bridged nucleic acid monomer (such as a locked nucleic acid [LNA] monomer), a peptide nucleic acid (PNA) monomer, a universal nucleoside, or a combination of any of these. Further suitable nucleotide analogues are described below.

Nucleotide analogues suitable for use in the oligonucleotide may have enhanced binding affinity compared with their native DNA or RNA counterparts. For example, for the purposes of binding to RNA, the analogue may have an enhanced affinity for RNA such as a 2′-modified RNA analogue (for example, 2-O-Methyl RNA, 2′ Fluoro-RNA and 2-O-ethyl RNA). Bridged nucleic acid monomers comprise a linkage from the 2′ position in the ribose ring to the 4′-position. Bridged nucleic acid monomers (for example, LNA monomers) are suitable analogues for oligonucleotides intended for binding and recognition of DNA targets. Oligonucleotides comprising both one or more 2′-modified RNA analogues and one or more bridged nucleic acid monomers are also encompassed.

An oligonucleotide comprising PNA, and a chimeric oligonucleotide comprising PNA and DNA, are suitable for binding to a DNA target. Due to lower solubility than other analogues, PNA oligonucleotides longer than 20 bases are currently difficult to manufacture so PNA probes may be shorter and the discontinuities may be position closer to the portion junction (for example, 3 to 4 bases rather than 8 to 12 bases, as described further below). Chimeric DNA-PNA oligonucleotides may be longer.

The oligonucleotide may comprise a label, for example a fluorescent tag, a mass tag, biotin, an enzyme, and/or a nanoparticle. The label may include a sequence which is non-complementary to the target nucleic acid.

The oligonucleotide may be in the form of a molecular beacon. As used herein, the term “molecular beacon” refers to a single-stranded oligonucleotide that form a stem-and-loop (also known as “hairpin”) structure under suitable conditions known in the art. The loop contains a probe sequence that is complementary to a target nucleic acid sequence, and the stem is formed by annealing—in the absence of target—of complementary arm sequences located at either side of the probe sequence. A fluorescent tag is linked (for example, covalently linked) at or near the end of one arm and a corresponding non-fluorescent quencher is linked (for example, covalently linked) to the end of the other arm. Examples of suitable corresponding fluorophore—non-fluorescent quencher partners include fluorescein as a fluorophore and DABCYL or Methyl Red as the quencher, ROX as the fluorophore and DABCYL or Methyl Red as the quencher. For a review, see Marras et al. (2002, Nucleic Acids Res. 30(21): e122).

A molecular beacon does not fluoresce when it is free in solution because the stem places the fluorophore sufficiently close to the non-fluorescent quencher that they transiently share electrons, eliminating the ability of the fluorophore to fluoresce. When the molecular beacon hybridises to a nucleic acid strand containing the target nucleic acid sequence, it undergoes a spontaneous conformational reorganisation that forces the stem to dissociate and the fluorophore and the quencher to move away from each other, thus allowing the molecular beacon to fluorescence (see Tyagi & Kramer, 1996, Nat. Biotech. 14: 303-308; Tyagi et al., 1998, Nat. Biotech. 16: 49-53; Kostrikis et al., 1998, Science 279: 1228-1229). Molecular beacons may comprise nucleotide analogues such as LNA or 2′-O-methyl RNA (Wang et al., 2005, J Am Chem Soc. 127(45): 15664-15665; Tsourkas et al., 2003, Nucleic Acids Res. 31(6): 5168-5174).

The oligonucleotide may have a 5′ to 3′ structure comprising the first portion, the portion junction and the second portion, where the first portion has a 5′ to 3′ structure: 1 to 45 nucleotides (for example, 5 to 12 nucleotides), a discontinuity, and 2 to 12 nucleotides, and the second portion has a 5′ to 3′ structure: 2 to 12 nucleotides, a discontinuity, and 1 to 45 nucleotides (for example, 5 to 12 nucleotides).

The oligonucleotide may be up to about 100 nucleotides in length, for example about 40 to 70 nucleotides, less than 40 nucleotides, about 30 nucleotides, less than 20 nucleotides, or at least about 16 nucleotides in length.

The target nucleic acid of the invention may be an mRNA molecule.

For example, the mRNA molecule may be a splice variant of a gene, the nucleic acid variant being an alternative splice variant of the same gene.

The portion junction of the oligonucleotide may correspond in position with the domain junction in the target nucleic acid. Where the target nucleic acid is a splice variant, the domain junction corresponds in position with a splice boundary.

Alternatively, the target nucleic acid may be a chimeric gene or its expressed mRNA.

Typically, although the first domain of the target nucleic acid has a sequence which is conserved with the first sequence in the nucleic acid variant, the first domain and second domain of the target nucleic acid in combination provide a unique sequence which may be detected specifically by an oligonucleotide of the present invention. For example, the first domain sequence and the first sequence in the nucleic acid variant may be a shared exon or a shared gene sequence or part thereof. The second domain adjacent the first domain renders the combined sequence formed in the target nucleic acid unique. The second domain may be a sequence (for example, an intron) which is not conserved between the target nucleic acid and its variant. Alternatively, the sequence of the second domain may itself be conserved between the target nucleic acid and its variant, but here the target nucleic acid has a unique sequence due to the positioning of the first domain adjacent the second domain in the target nucleic acid; a conserved second domain is positioned elsewhere in the variant.

The target nucleic acid in another aspect of the invention is a chromosome or any portion thereof.

The oligonucleotide may in one aspect be for use in detection of the target nucleic acid, for example by in situ hybridisation.

The oligonucleotide may additionally or alternatively be for use in an array.

Particularly when used for the detection of a target nucleic acid, and/or in an array, the oligonucleotide of the invention is also referred to herein as a “probe” or an “oligonucleotide probe”.

In another aspect, the oligonucleotide may be an antisense oligonucleotide.

The antisense oligonucleotide may comprise nucleotides with enhanced nuclease resistance and enhanced binding affinity such as 2′-O-methyl analogues, 2′-O-methoxyethoxy (2′-MOE) analogues or 2′-deoxy-2′-fluoro-D-arabinose analogues.

The antisense oligonucleotide may comprise, in addition to the first and second portions, an RNase H-recruiting portion that can recruit RNase H to a duplex of the antisense oligonucleotide with RNA. The RNase H-recruiting portion the antisense oligonucleotide may for example have a length of between 5 and 7 nucleotides. The antisense oligonucleotide may additionally comprise stretches of nuclease resistant nucleotides with enhanced binding affinity flanking the RNase H-recruiting portion, for example on both the 3′ and 5′ sides of the RNase H-recruiting portion.

According to a further aspect of the invention, there is provided a set of oligonucleotides comprising two or more oligonucleotides, in which a first oligonucleotide is as defined herein and a second or further oligonucleotide is as defined herein except that the second or further oligonucleotide is hybridisable with greater affinity to one or more nucleic acid variants of the target nucleic acid.

The set of oligonucleotides may be for use in simultaneous or sequential detection of the target nucleic acid and the one or more nucleic acid variants.

The set of oligonucleotides of the invention may be attached to a solid surface such as an array (or microarray) or a magnetic bead.

Also provided according to the invention is an array (or microarray) comprising an oligonucleotide or a set of oligonucleotides as defined herein.

In another aspect, there is provided a method for detecting the presence or absence of a target nucleic acid in a sample, the target nucleic acid comprising a first domain adjacent a second domain, the first domain having a sequence which is conserved with a first sequence in a nucleic acid variant of the target nucleic acid, comprising the steps of:

(i) labelling nucleic acids in the sample;

(ii) contacted nucleic acids labelled in step (i) with an oligonucleotide of the invention as defined herein under conditions which allow hybridisation of the oligonucleotide to the target nucleic acid to form a duplex molecule;

(iii) optionally washing the duplex molecule; and

(iv) detecting the presence or absence of the target nucleic target nucleic acid by determining the presence of absence of label bound to the oligonucleotide.

In a further aspect, there is provided a method for detecting the presence or absence of a target nucleic acid and one or more nucleic acid variants of the target nucleic acid in a sample, the target nucleic acid comprising a first domain adjacent a second domain, the first domain having a sequence which is conserved with a first sequence in the or each nucleic acid variant, comprising the steps of:

(i) labelling nucleic acids in the sample;

(ii) contacted nucleic acids labelled in step (i) with a set of oligonucleotides of the invention as defined herein under conditions which allow hybridisation of the oligonucleotides to form duplex molecules with the target nucleic acid and the nucleic acid variants;

(iii) optionally washing the duplex molecules; and

(iv) detecting the presence or absence of the target nucleic acid and the nucleic acid variants by determining the presence of absence of label bound to the oligonucleotides.

In these methods, the or each oligonucleotide may be immobilised on a solid surface (for example a matrix, a planar surface, a bead such as a magnetic bead or a fluorescently encoded microparticle).

The invention may allow the simultaneous or sequential detection of multiple target nucleic acids and their variants. In one aspect, there is provided multiplexed detection of multiple targets using the oligonucleotide probes of this invention.

Also provided is a method for detecting the presence or absence of a target nucleic acid in a sample, the target nucleic acid comprising a first domain adjacent a second domain, the first domain having a sequence which is conserved with a first sequence in a nucleic acid variant of the target nucleic acid, comprising the steps of:

(i) labelling an oligonucleotide of the invention as defined herein (or providing an oligonucleotide as defined herein which has been labelled);

(ii) contacting the sample with the oligonucleotide labelled in step (i) under conditions which allow hybridisation of the oligonucleotide to the target nucleic acid to form a duplex molecule;

(iii) optionally washing the duplex molecule; and

(iv) detecting the presence or absence of the target nucleic target nucleic acid by determining the presence of absence of label bound to the oligonucleotide.

Further provided is a method for detecting the presence or absence of a target nucleic acid and one or more nucleic acid variants of the target nucleic acid in a sample, the target nucleic acid comprising a first domain adjacent a second domain, the first domain having a sequence which is conserved with a first sequence in the or each nucleic acid variant, comprising the steps of:

(i) labelling a set of oligonucleotides of the invention as defined herein (or providing a set of oligonucleotides as defined herein which has been labelled);

(ii) contacting the sample with oligonucleotides labelled in step (i) under conditions which allow hybridisation of the oligonucleotides to form duplex molecules with the target nucleic acid and the nucleic acid variants;

(iii) optionally washing the duplex molecules; and

(iv) detecting the presence or absence of the target nucleic acid and the nucleic acid variants by determining the presence of absence of label bound to the target nucleic acid and the nucleic acid variants.

The methods defined above may be in situ hybridisation methods, in which case the or each oligonucleotide may be labelled with a fluorescent tag, a mass tag, biotin, an enzyme, and/or a nanoparticle.

The method of this invention may further comprise the step of quantifying the amount of target nucleic acid or nucleic acid variant by measuring the amount of bound label.

The optional washing steps of the methods of the invention serve to destabilise and remove incorrectly hybridised duplexes or non-specifically adsorbed labelled nucleic acid. Alternatively, detection methods such as surface-sensitive methods that can discriminate between the presence and absence of a duplex formed between the oligonucleotide and target may be used. Suitable detection methods that do not require a wash step after hybridisation include surface plasmon resonance and evanescent wave fluorescence detection.

In certain embodiments of the methods of the invention, the nucleic acids in the sample may be amplified prior to hybridisation. Suitable methods of amplification include PCR, Strand Displacement Amplification (SDA) and Rolling Circle Amplification (RCA). Methods for amplification of mRNA, such as in vitro transcription (IVT), may be used.

Conditions in step (ii) of the methods of the invention may be optimised for salt concentration and/or temperature such that the or each oligonucleotide binds with enhanced specificity to its target nucleic acid. As elaborated in the experimental section below, appropriate selection of the salt concentration and/or specific temperature or range of temperatures for formation of duplex molecules can allow enhanced discrimination of the oligonucleotide between a target nucleic acid and one or more target nucleic acid variants. In one embodiment, hybridisation between the or each oligonucleotide and its target nucleic acid may be simulated using appropriate software to determine optimal salt concentration and/or temperature to enhance specificity. For example, software such as DINAMelt (Markham & Zuker, 2005, Nucleic Acids Res. 33(Web Server issue): W577-81) may be used. DINAMelt is a web application that exploits the open source software tool, UNAFoId (Markham, N. R. & Zuker, M. (2008) In Keith, J. M., editor, Bioinformatics, Volume II. Structure, Functions and Applications, No. 453, in Methods in Molecular Biology, chapter 1, pages 3-31. Humana Press, Totowa, N.J. ISBN 978-1-60327-428-9).

Additionally provided according to the invention is a kit comprising one or more oligonucleotides of the invention as defined herein or a set of oligonucleotides of the invention as defined herein. The oligonucleotides of the kit may for example be provided in an unbound form (for example, in solution or as a lyophilised powder) for use in the hybridisation to targets in solution or to tissue sections or to cellular samples immobilised on a surface such as a glass slide. In an alternative embodiment, the oligonucleotides of the kit may be provided as an array of immobilised oligonucleotides in which, for example, the oligonucleotides are immobilised at pre-determined discrete locations on an array surface.

In a further aspect of the invention, there is provided an oligonucleotide (such as an oligonucleotide probe) which is hybridisable with greater affinity to a target nucleic acid than to a nucleic acid variant of the target nucleic acid, the target nucleic acid comprising a first domain and a second domain flanking a domain junction, the first domain having a sequence which is conserved with a first sequence in the nucleic acid variant, wherein the oligonucleotide has a first portion and a second portion flanking a portion junction, the first portion being complementary or complementary in part to the first domain and the second portion being complementary or complementary in part to the second domain, and wherein the first portion and the second portion have substantially the same Tₘ.

As noted above, by “substantially the same” is meant that the Tₘ of the first portion is identical with, or up to 10% different from, for example up to 9%, 8%, 7%, 6%, 5%, 4%, 3%, 2% or 1% different from, the Tₘ of the second portion.

In this further aspect of the invention, the first and/or second portions of the oligonucleotide may be completely complementary to the first and second domains, respectively. Alternatively, the first portion may comprise at least a first discontinuity relative to the first domain and/or the second portion may comprise at least a second discontinuity relative to the second domain, each discontinuity comprising or consisting of a sequence mismatch and/or a non-nucleotide spacer.

It has been found that discrimination of the oligonucleotide between the target nucleic acid and its target nucleic acid variant(s) may be enhanced by using an oligonucleotide where the first and second portions have substantially the same Tₘ, irrespective of whether or not the oligonucleotide comprises one or more discontinuities relative to the target nucleic acid.

Other features of the oligonucleotide according to this further aspect of the invention, as well as kits and methods utilising the oligonucleotide, are as described herein for other aspects of the invention, mutatis mutandis.

The invention will now be described in further detail and with reference to particular non-limiting examples and the following drawings, in which:

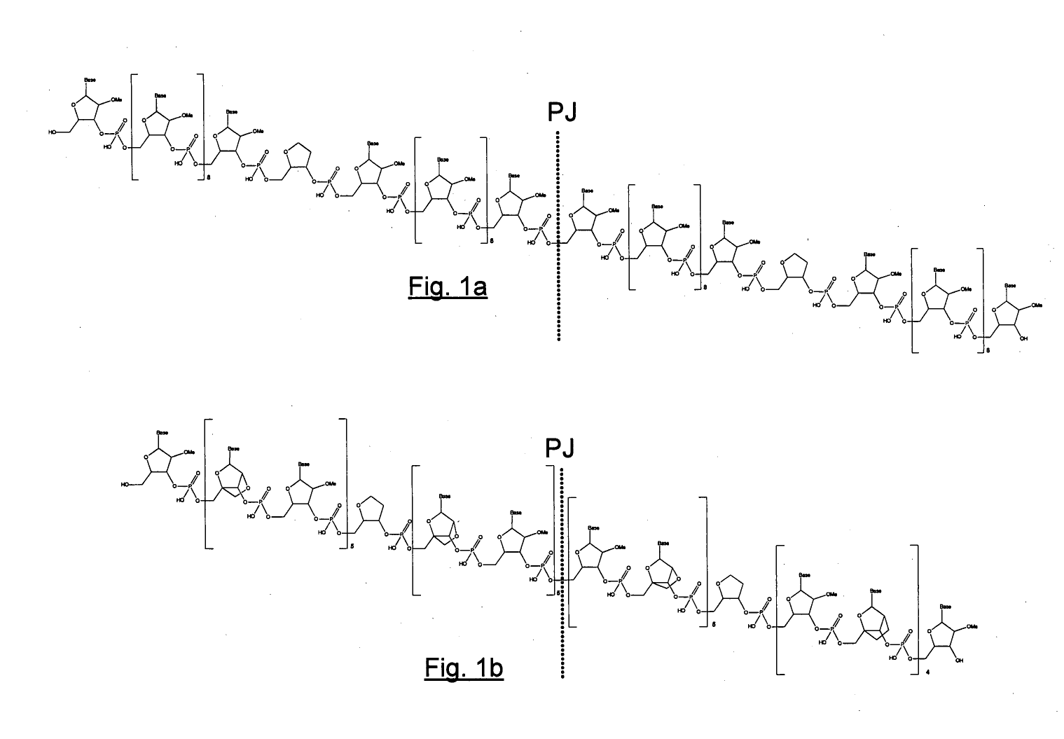

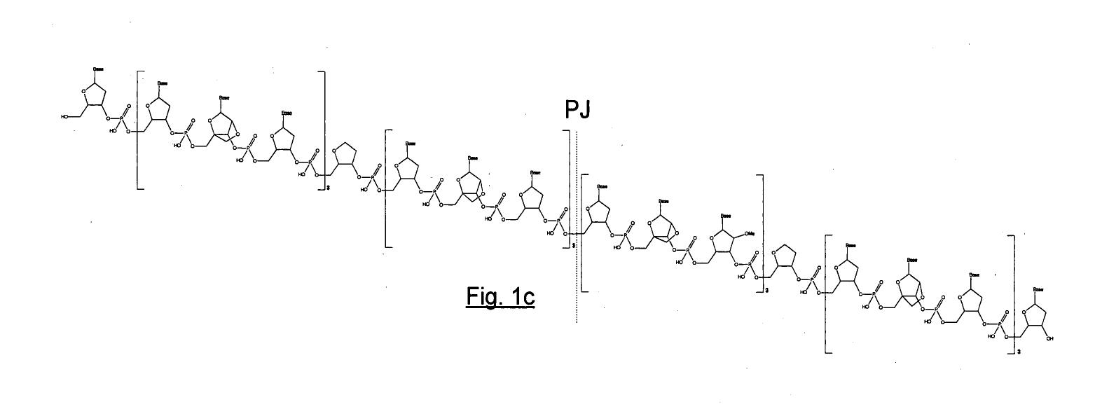

FIGS. 1a-c illustrate three forms of oligonucleotides according to the invention, employing nucleic acid analogues and comprising abasic spacer discontinuities. FIG. 1a shows an oligonucleotide comprising 2-O-methyl modified nucleotides, FIG. 1b shows an oligonucleotide comprising 2-O-methyl modified nucleotides interspersed with LNA nucleotides, and FIG. 1c shows an oligonucleotide comprising DNA nucleotides interspersed with LNA nucleotides;

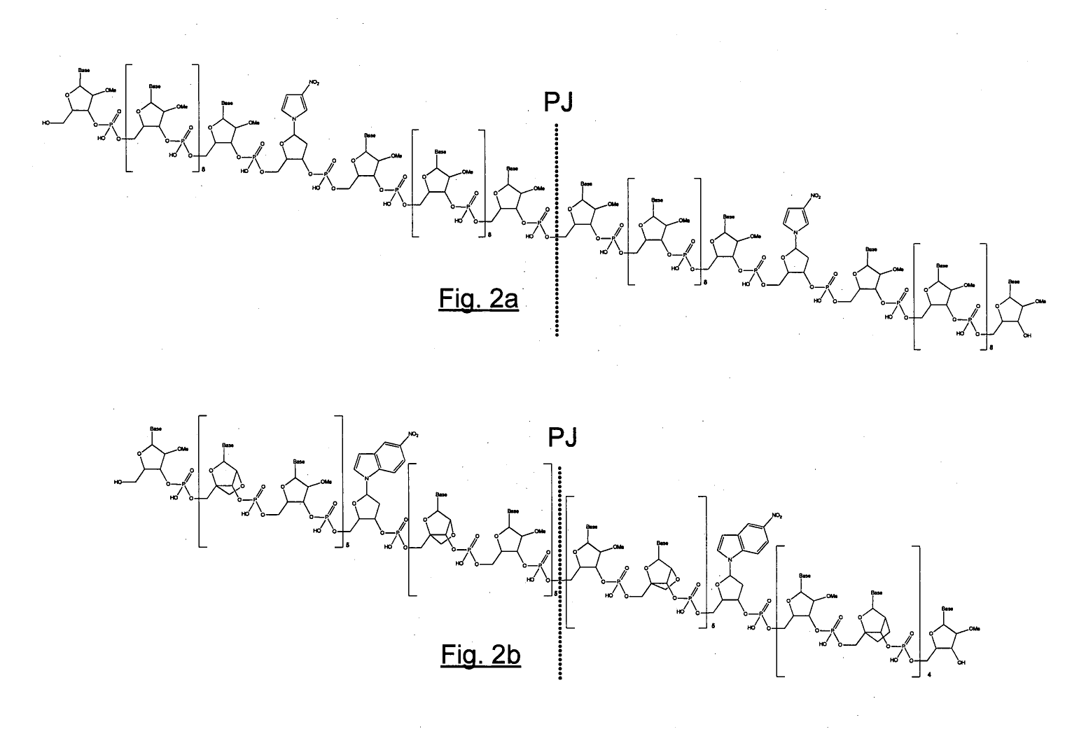

FIGS. 2a-b illustrate two further forms of oligonucleotides, where each discontinuity comprises a universal nucleotide. FIG. 2a shows an oligonucleotide comprising 2-O-methyl modified nucleotides, while FIG. 2b shows an oligonucleotide comprising 2-O-methyl modified nucleotides interspersed with LNA nucleotides;

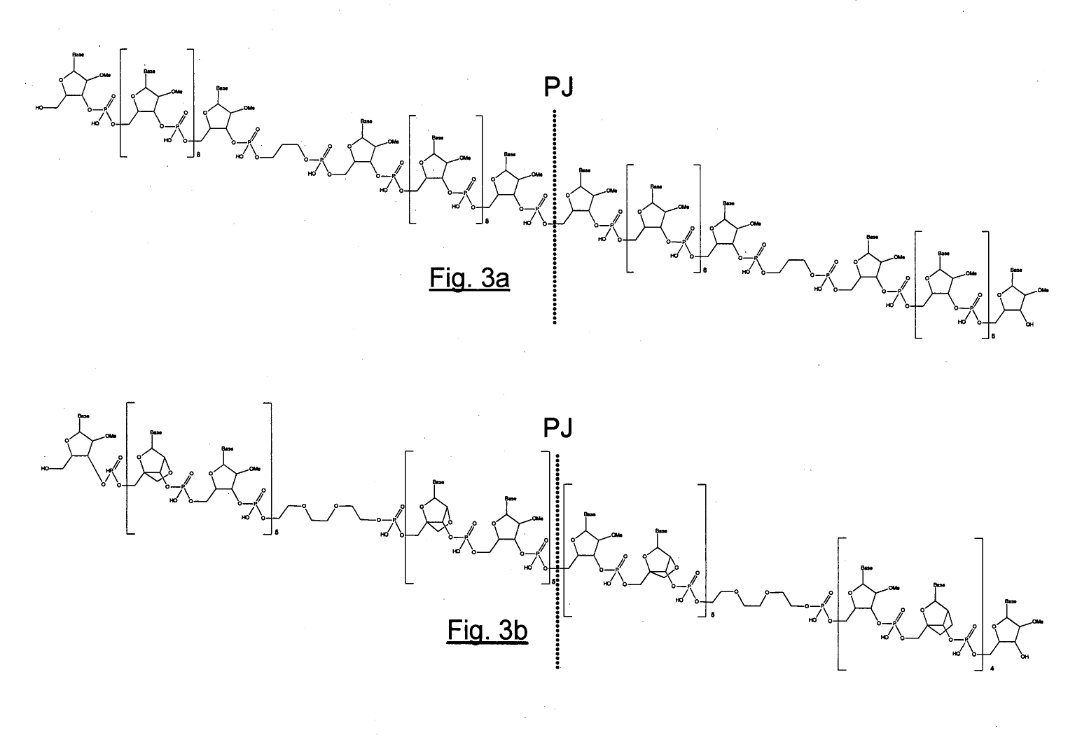

FIGS. 3a-c illustrate another three forms of oligonucleotides according to the invention, employing nucleic acid analogues and comprising simple linker discontinuities. FIG. 3a shows an oligonucleotide comprising 2-O-methyl modified nucleotides and with a discontinuity that comprises a 3-carbon spacer (C3 spacer), FIG. 3b shows an oligonucleotide comprising 2-O-methyl modified nucleotides interspersed with LNA nucleotides and with a discontinuity that comprises a 12-atom ethylene glycol linker, and FIG. 3c shows an oligonucleotide comprising DNA nucleotides interspersed with LNA nucleotides and with a discontinuity comprising an 18-atom ethylene glycol linker;

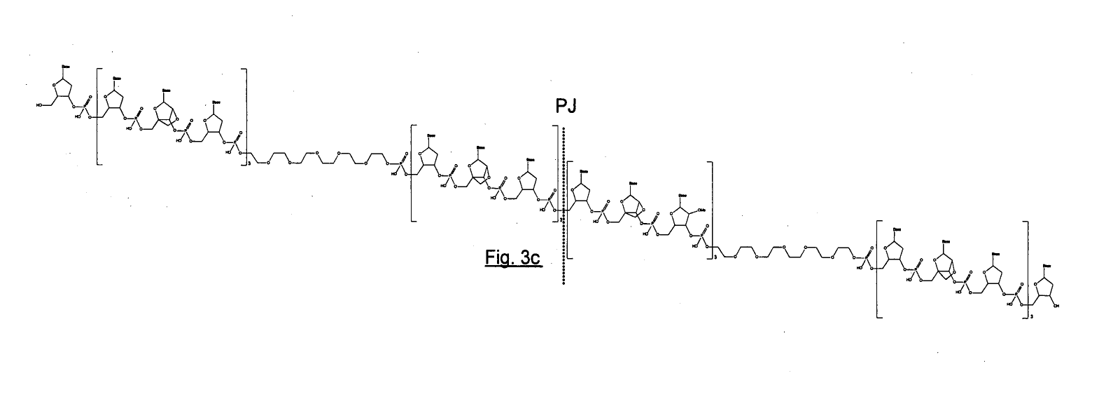

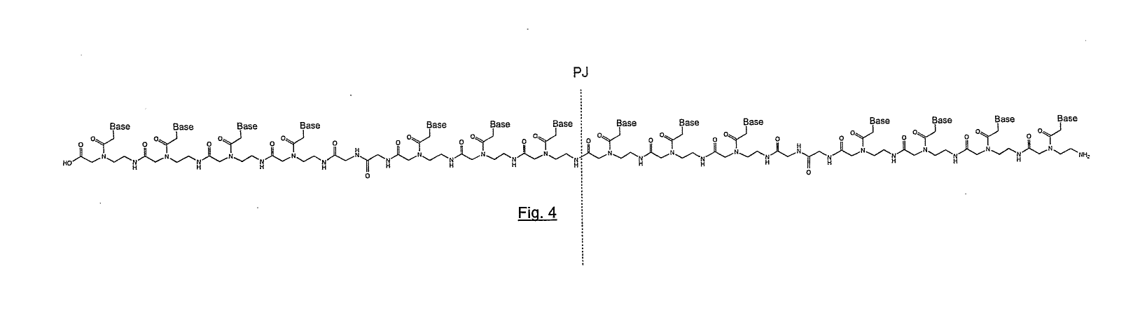

FIG. 4 illustrates a PNA oligonucleotide probe design with a discontinuity comprising an alpha amino acid linker;

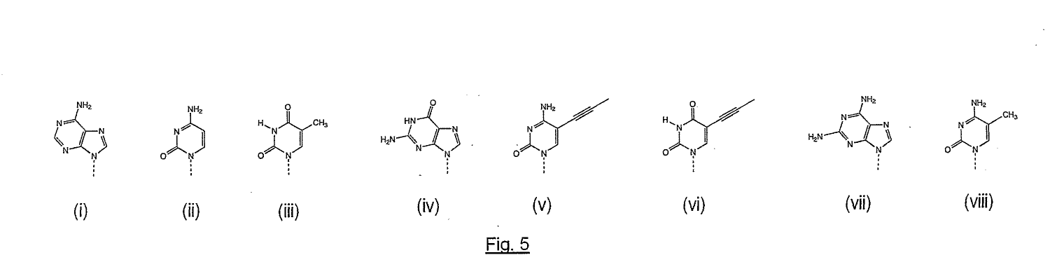

FIGS. 5(i)-(viii) illustrate different base options suitable for introduction into oligonucleotides of the invention;

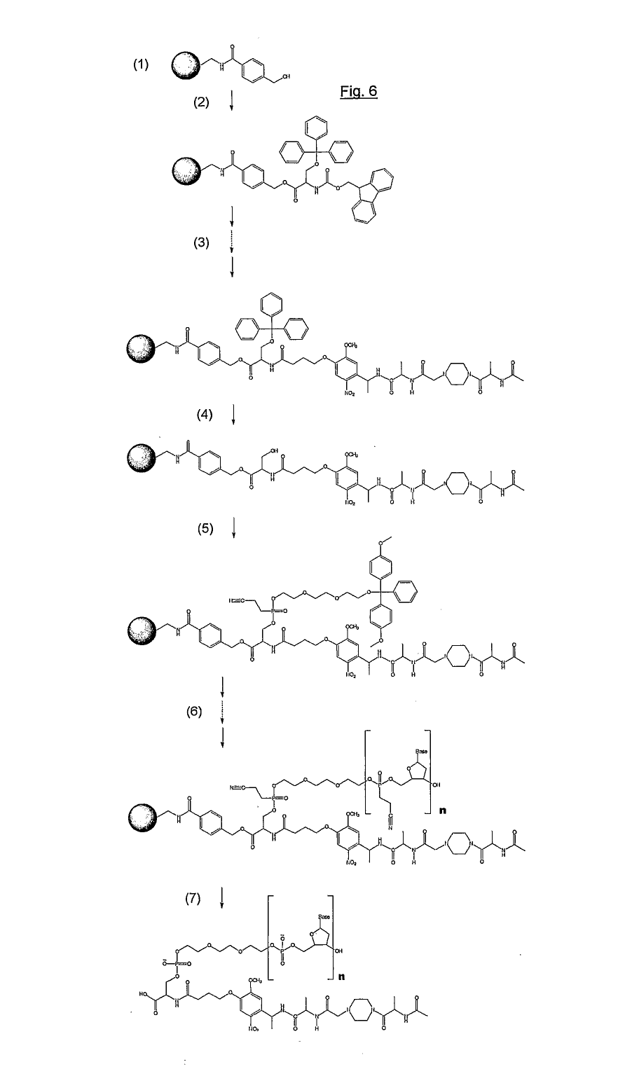

FIG. 6 is a schematic representation of a synthesis method for the preparation of singly labelled oligonucleotides;

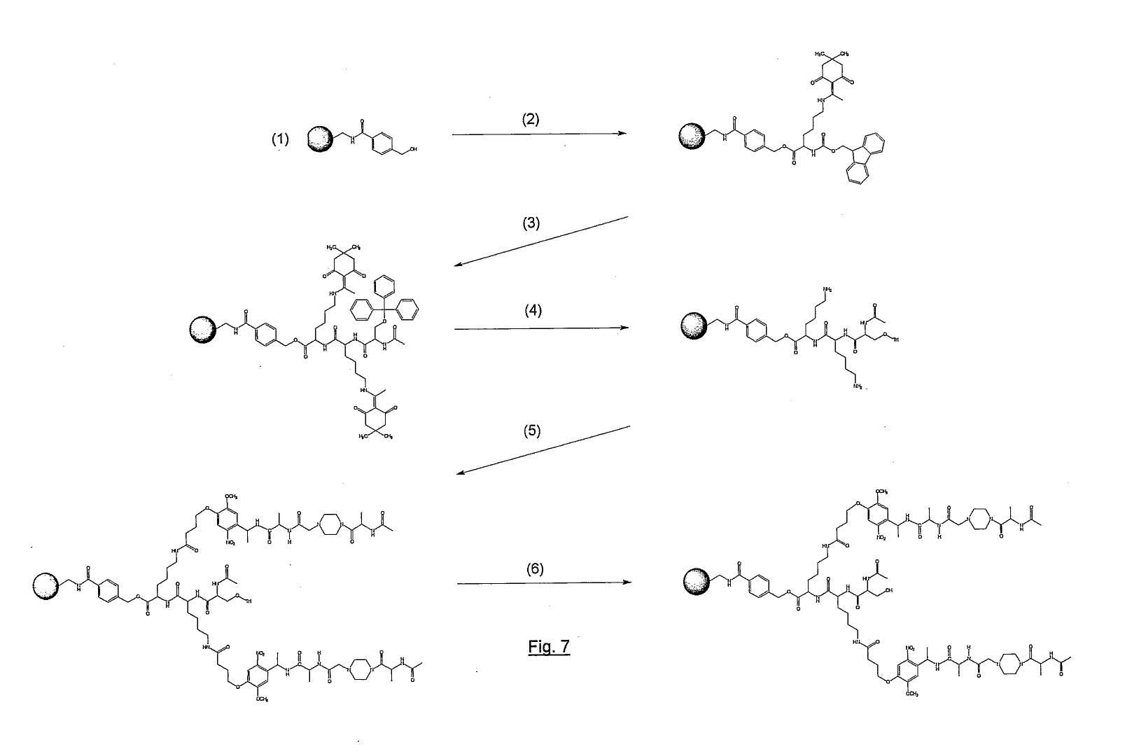

FIG. 7 (in two parts) is a schematic representation of a synthesis method for the preparation of multiply labelled oligonucleotides;

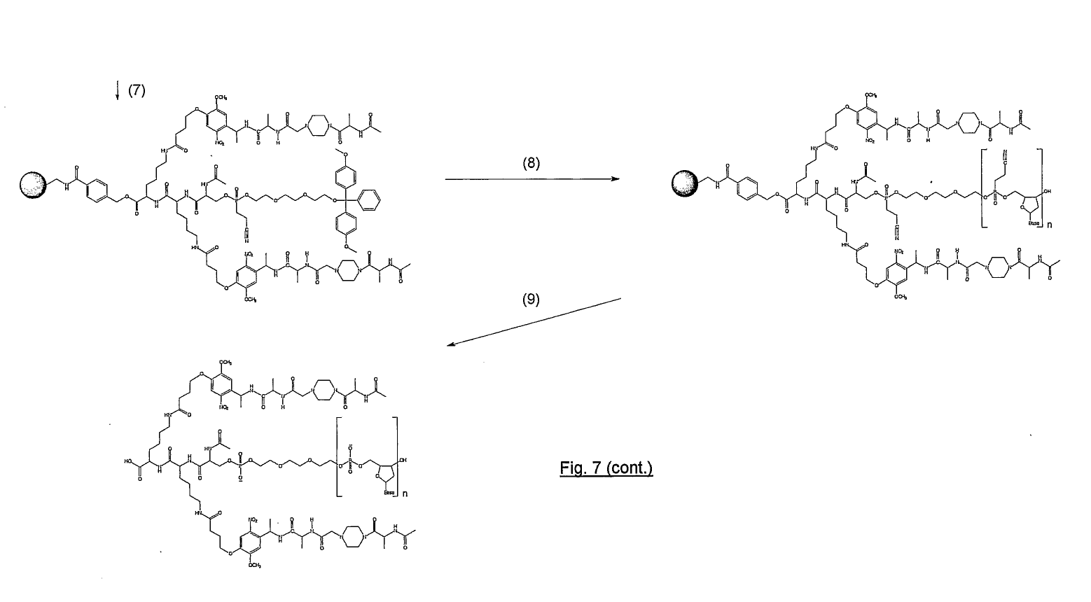

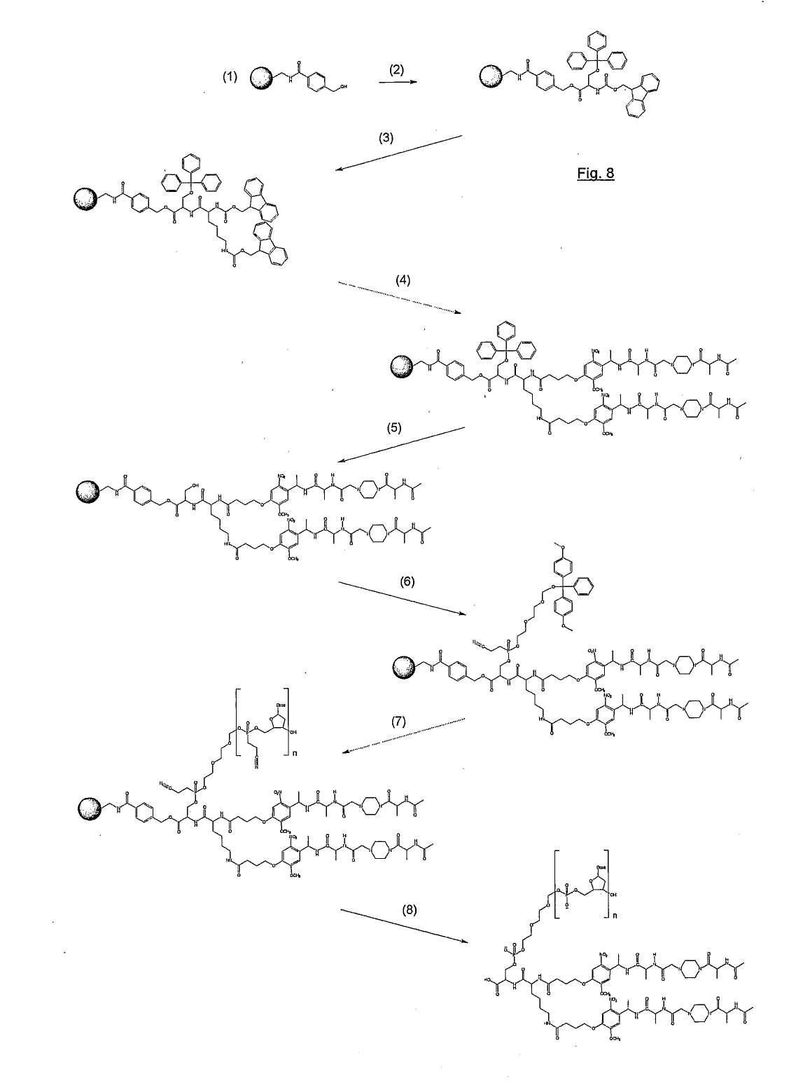

FIG. 8 is a schematic representation of an alternative synthesis method (compared to that shown in FIG. 7) for the preparation of multiply labelled oligonucleotides;

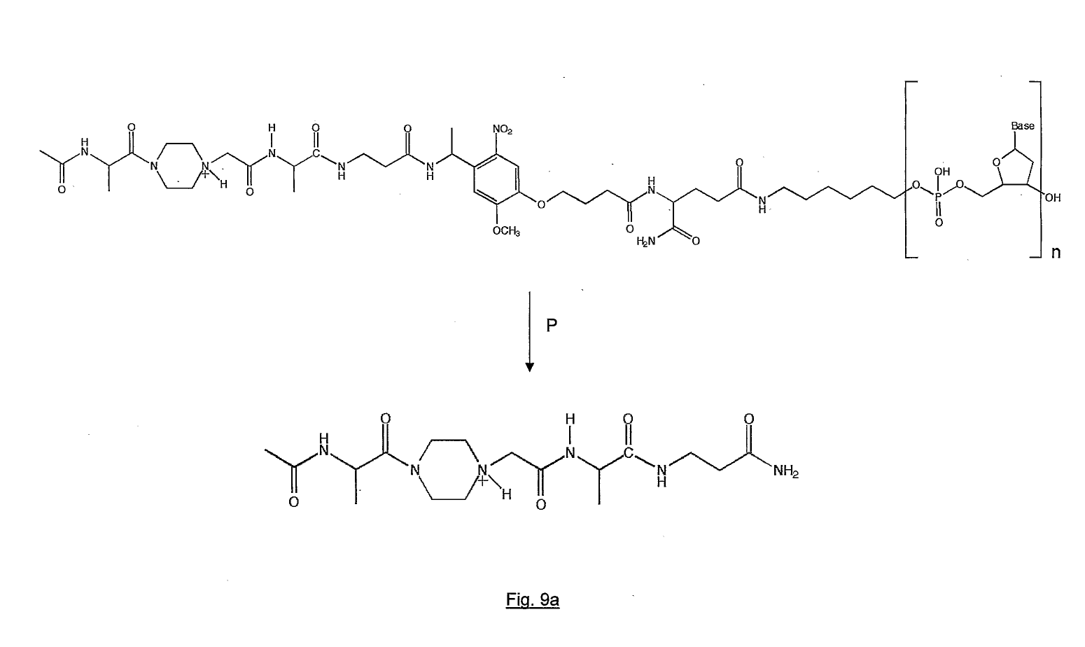

FIGS. 9a-b show a series of isobaric mass tag sets. FIG. 9a illustrates a mass tagged oligonucleotide probe based on the peptide (N)—Acetate—Valine—Piperazin-1-ylacetic acid—Alanine—Beta-alanine—(C), while FIG. 9b illustrates a set of 9 isobaric tags constructed by combinatorial rearrangement of the mass modifier amino acids, beta-alanine, alanine and valine, and isotopes of acetic acid in the tags;

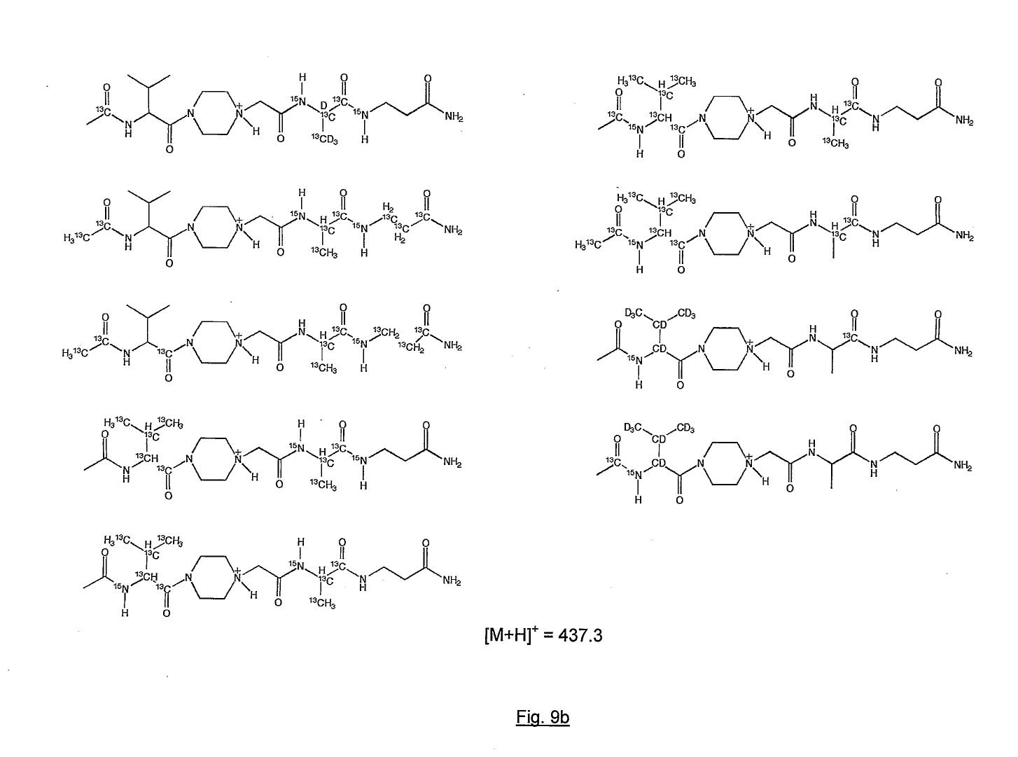

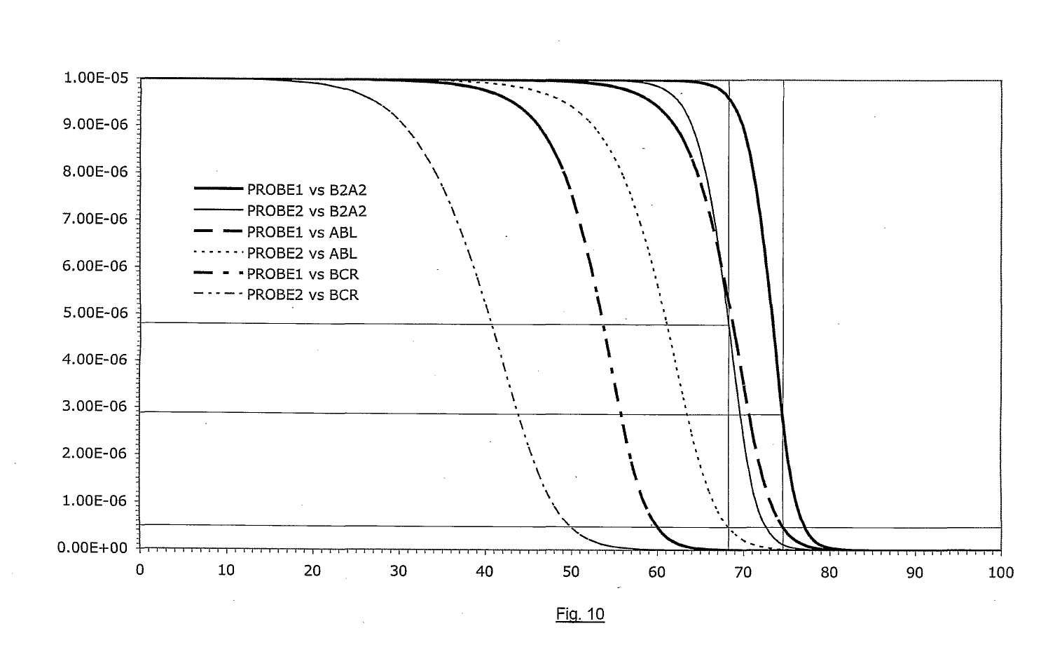

FIG. 10 is a graph showing melting curves of exemplar Probes 1 and 2 against a target nucleic acid B2A2 and variant nucleic acids ABL and BCR. The x-axis represents temperature in ° C. while the y-axis represents mole fraction;

FIG. 11 is a graph showing melting curves of exemplar Probes 3 and 4 against a target nucleic acid B2A2 and variant nucleic acids ABL and BCR. The x-axis represents temperature in ° C. while the y-axis represents mole fraction;

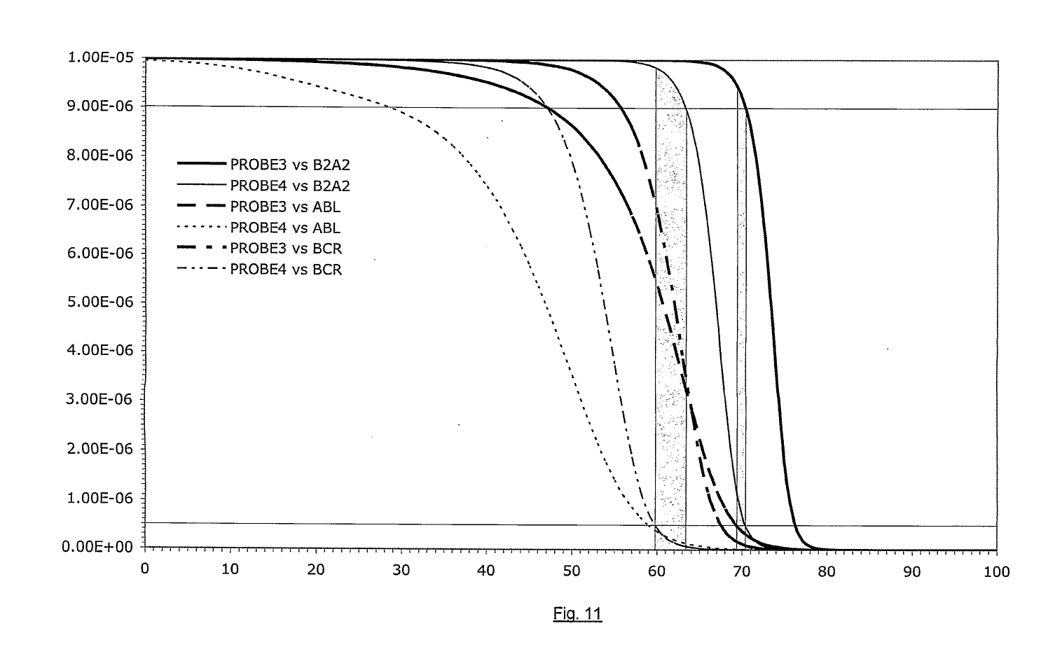

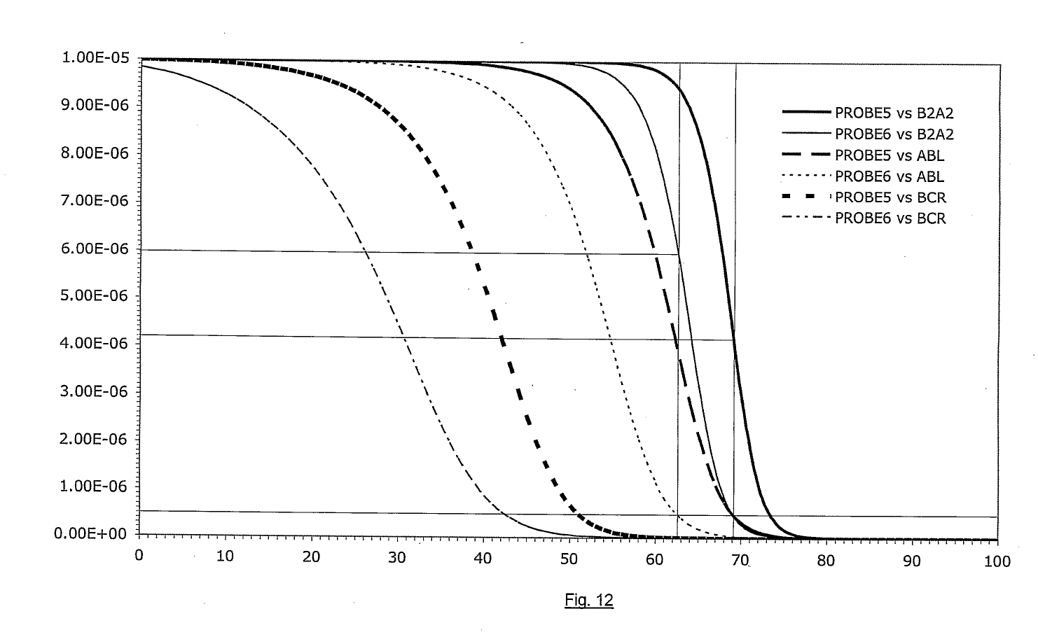

FIG. 12 is a graph showing melting curves of exemplar Probes 5 and 6 against a target nucleic acid B2A2 and variant nucleic acids ABL and BCR. The x-axis represents temperature in ° C. while the y-axis represents mole fraction;

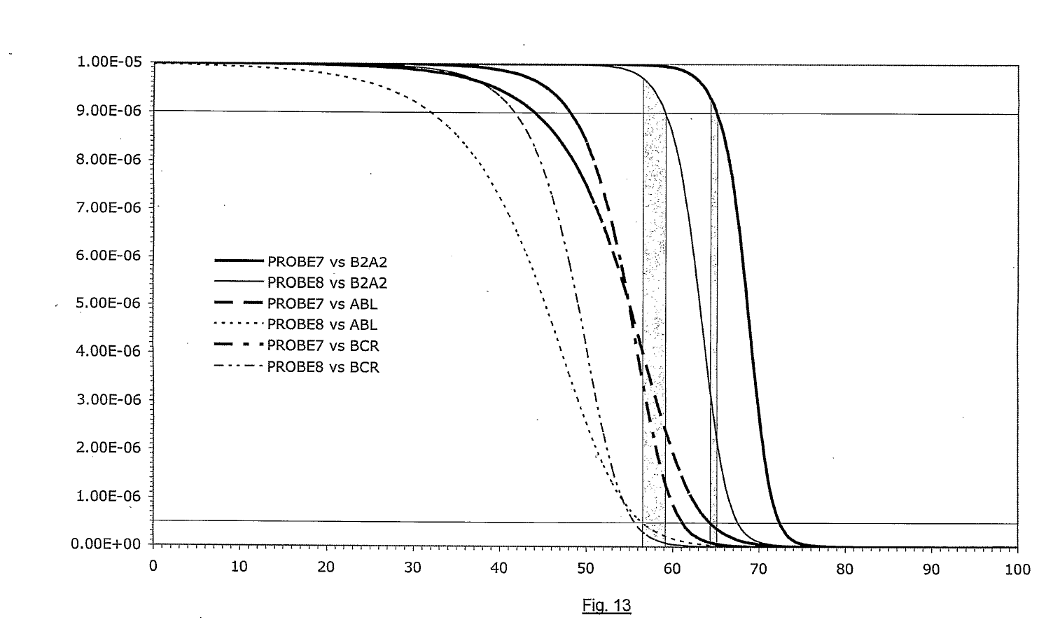

FIG. 13 is a graph showing melting curves of exemplar Probes 7 and 8 against a target nucleic acid B2A2 and variant nucleic acids ABL and BCR using 50 mM salt. The x-axis represents temperature in ° C. while the y-axis represents mole fraction;

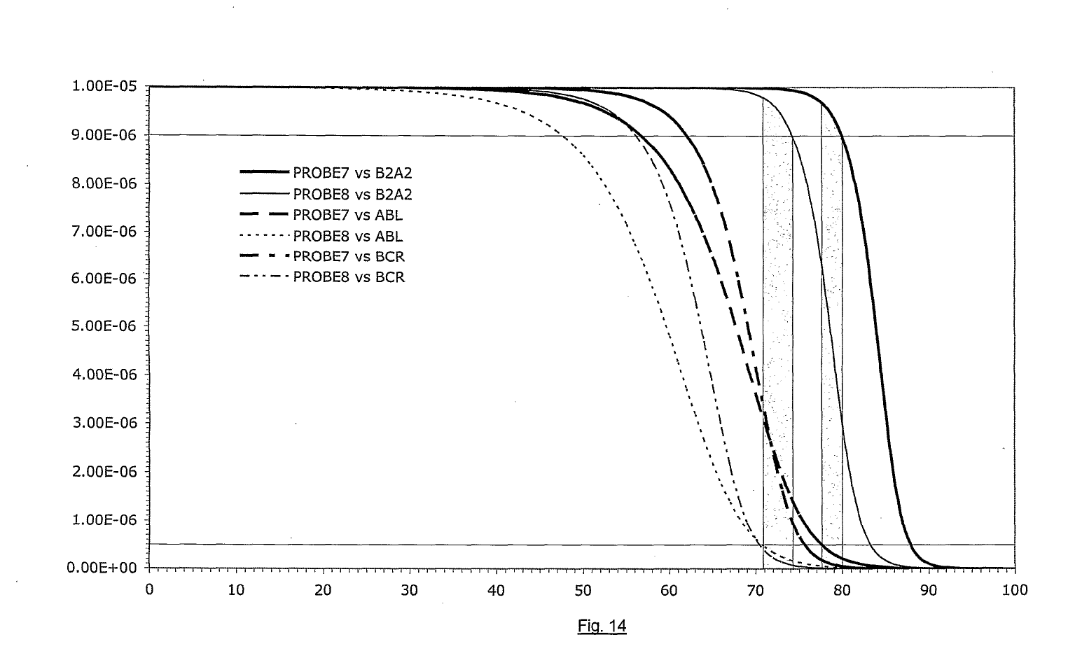

FIG. 14 is a graph showing melting curves of exemplar Probes 7 and 8 against a target nucleic acid B2A2 and variant nucleic acids ABL and BCR using 1 M salt. The x-axis represents temperature in ° C. while the y-axis represents mole fraction;

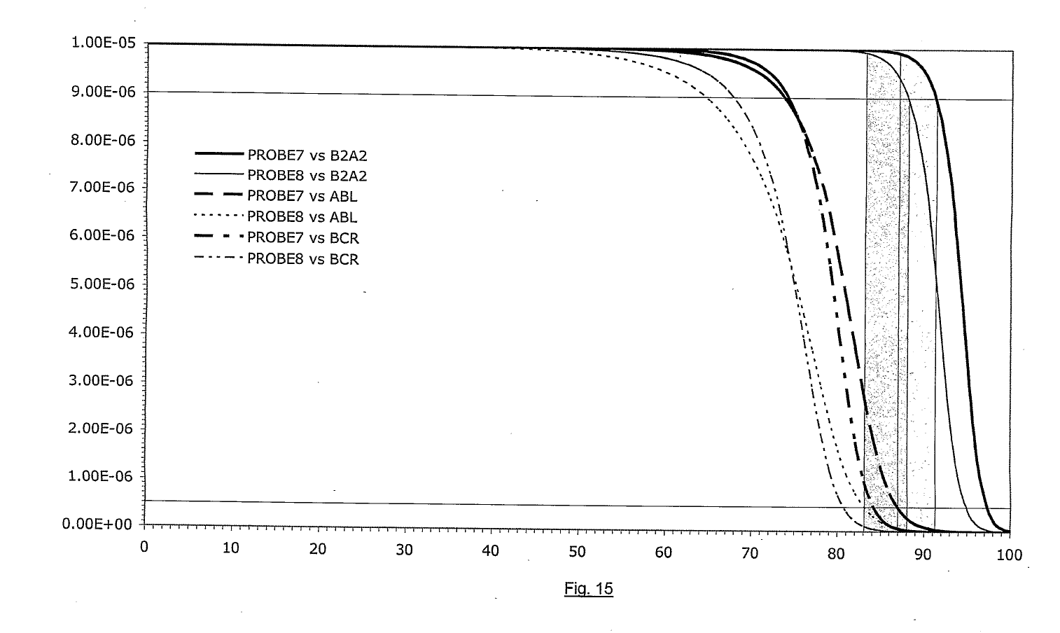

FIG. 15 is a graph showing melting curves of exemplar Probes 7 and 8 against a target nucleic acid B2A2 and variant nucleic acids ABL and BCR using 1 M salt but where the probes, target and variant nucleic acids are RNA rather than DNA. The x-axis represents temperature in ° C. while the y-axis represents mole fraction;

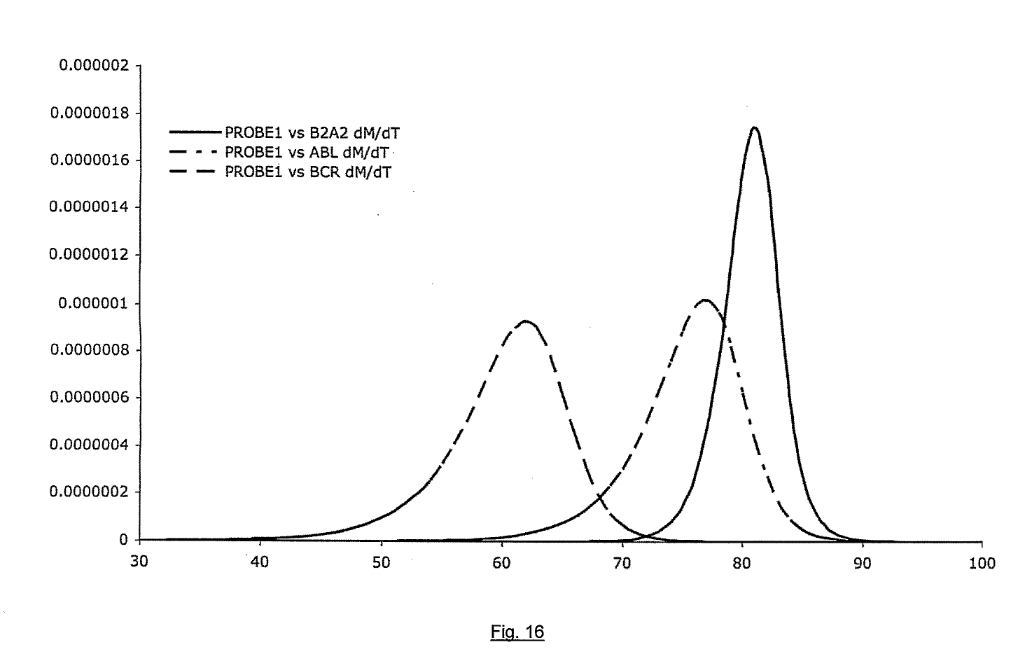

FIG. 16 is a graph showing a mole fraction difference curve (dM/dT curve) that corresponds to the melting curves for PROBEI shown in FIG. 10. The x-axis represents temperature in ° C. while the y-axis represents mole fraction difference (dM/dT); and

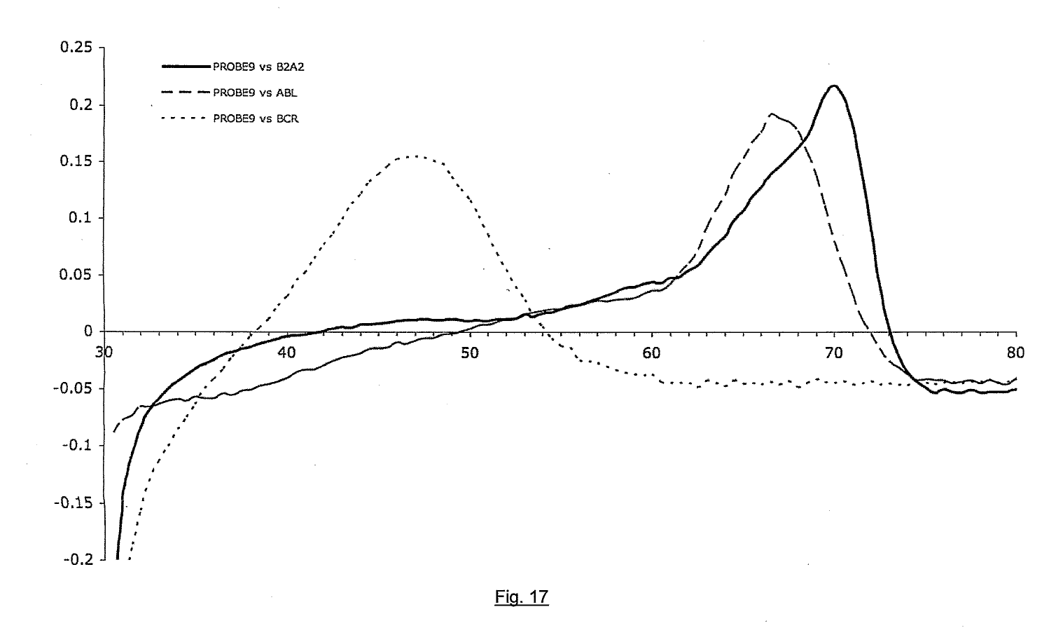

FIG. 17 is a graph showing a typical set of fluorescence difference curves (dF/dT curves) for the hybridization of PROBE9 to its intended target B2A2_FRET and corresponding hybridizations to ABL_FRET and BCR_FRET targets. The x-axis represents temperature in ° C. while the y-axis represents fluorescence difference (dF/dT).

As used herein, the terms “nucleic acid”, “oligonucleotide”, “oligonucleotide probe”, “probe” or similar wording refer to polymers composed of naturally occurring nucleotides, polymers composed of synthetic or modified nucleotides (i.e. nucleotide analogues), or a combination of natural, synthetic and/or modified nucleotides. Furthermore, these terms encompass polymers including non-nucleotide structures such as linkers. A polynucleotide that is a RNA or DNA may include naturally occurring moieties such as the naturally occurring bases and ribose or deoxyribose rings, or they may be composed of synthetic or modified moieties as described elsewhere herein. The linkage between nucleotides is commonly the 3′-5′ phosphate linkage, which may be a natural phosphodiester linkage, a phosphothioester linkage, and other synthetic linkages. Examples of modified backbones include phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates. Additional linkages include phosphotriester, siloxane, carbonate, carboxymethylester, acetamidate, carbamate, thioether, bridged phosphoramidate, bridged methylene phosphonate, bridged phosphorothioate and sulfone internucleotide linkages. Other polymeric linkages include 2′-5′ linked analogs of these (see for example U.S. Pat. No. 6,503,754 and U.S. Pat. No. 6,506,735). The monosaccharide may be modified by being, for example, a pentose or a hexose other than a ribose or a deoxyribose. The monosaccharide may also be modified by substituting hydryoxyl groups with hydro or amino groups, by esterifying additional hydroxyl groups, and so on. Further backbone modifications suitable for use in the invention are described below.

Unless otherwise evident from the context, as used herein the terms nucleic acid and nucleotide are synonymous and interchangeable.

As used herein, a target may be a nucleic acid target such as a chromosome or any portion thereof, an expressed nucleic acid, particularly mRNA, a recombinant nucleic acid molecule such as a plasmid or oligonucleotide, or another nucleic acid fragment. A nucleic acid target may be naturally occurring or synthetic or a combination of the two. The target may be of any length provided that it is able to complement a probe of this invention. The nucleic acid target may be DNA or RNA. When the target is DNA, it is understood that the DNA is hybridisable in the methods of the invention. For example, the DNA may be provided for use in the methods in a denatured or single-stranded form capable of hybridising to a single-stranded oligonucleotide probe.

As used herein, the term “complementary” refers to Watson-Crick or Hoogsteen base pairing between nucleotides units of a nucleic acid molecule. The term “complementary” and similar words relate to the ability of a first nucleic acid base in one strand of a nucleic acid or oligonucleotide to interact specifically only with a particular second nucleic acid base in a second strand of a nucleic acid or oligonucleotide. By way of non-limiting example, if the naturally occurring bases are considered, A and T or U interact with each other, and G and C interact with each other.

The terms “hybridise”, “hybridisation” and similar words relate to a process of forming a nucleic acid or oligonucleotide duplex by causing strands with complementary sequences to interact with each other. The interaction occurs by virtue of complementary bases on each of the strands specifically interacting to form a pair. The ability of strands to hybridise to each other depends on a variety of conditions, as known in the art. Nucleic acid or oligonucleotide strands may be stated to hybridise with each other when a sufficient number of corresponding positions in each strand are occupied by nucleotides that can interact with each other. For reasons described herein, the sequences of strands forming a duplex need not be 100% complementary (or identical) to each other to be specifically hybridisable.

As used herein, an oligonucleotide sequence which is “conserved” with another oligonucleotide sequence indicates that the sequences may share at least 60% sequence identity with each other over a given domain or portion or region of the sequences, for example up to 65%, 70%, 75%, 80%, 85%, 90%, 95%, 99 or 100% sequence identity.

Sequence identity between oligonucleotide sequences can be determined by comparing an alignment of the sequences. When an equivalent position in the compared sequences is occupied by the same base, then the molecules are identical at that position. Scoring an alignment as a percentage of identity is a function of the number of identical bases at positions shared by the compared sequences. When comparing sequences, in one aspect no gaps are introduced during the determination of sequence identity. In another aspect, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include the MatGat program (Campanella et al., 2003, BMC Bioinformatics 4: 29), the Gap program (Needleman & Wunsch, 1970, J. Mol. Biol. 48: 443-453) and the FASTA program (Altschul et al., 1990, J. Mol. Biol. 215: 403-410). MatGAT v2.03 is freely available from the site “http://bitincka.com/ledion/matgat/” and has also been submitted for public distribution to the Indiana University Biology Archive (IUBIO Archive). Gap and FASTA are available as part of the Accelrys GCG Package Version 11.1 (Accelrys, Cambridge, UK), formerly known as the GCG Wisconsin Package. The FASTA program can alternatively be accessed publically from the European Bioinformatics Institute (http://www.eblac.uk/fasta) and the University of Virginia (http://fasta.biotech.virginia.edu/fasta_www/cgi). FASTA may be used to search a sequence database with a given sequence or to compare two given sequences (see http://fasta.bioch.virginia.edu/fasta_www/cgi/search_frm2.cgi). Typically, default parameters set by the computer programs may be used when comparing sequences. The default parameters may change depending on the type and length of sequences being compared. A sequence comparison using the MatGAT program may use default parameters of Scoring Matrix=Blosum50, First Gap=16, Extending Gap=4 for DNA. A comparison using the FASTA program may use default parameters of Ktup=2, Scoring matrix=Blosum50, gap=−10 and ext=−2.

Generally, oligonucleotide sequences which are conserved with each other may hybridise under high stringency conditions. Typically, high stringency conditions may involve a wash in a 0.0165-0.0330 M NaCl buffer solution at a temperature of about 5-10° C. below the calculated or actual Tₘ of the probe (for example, about 65° C.). The buffer solution may, for example, be SSC buffer (0.15M NaCl and 0.015M tri-sodium citrate), with the high stringency wash taking place in 0.1× SSC buffer. Steps involved in hybridisation of nucleic acid sequences have been described for example in Sambrook et al. (1989; Molecular Cloning, Cold Spring Harbor Laboratory Press, Cold Spring Harbor).

The term “splice junction” refers to a site in mRNA where splicing to join an intron and an exon (or to join two exons in the case of trans-splicing) has taken place.

The oligonucleotide need not correspond to the full length of the target. The oligonucleotide may include sequences which are not generally complementary to the target.

Particularly in the context of oligonucleotides comprising sugar/phosphate backbones, including modified native dexoxyribose/phosphodiester backbones, a “discontinuity” is herein defined as an interruption in the chemical sequence of nucleotides with one or more non-base-pairing nucleotides (“mismatches”) and/or non-nucleotide “spacers”.

Various parameters relating to the oligonucleotides of the invention, including their preparation, structure and uses, are described further below.

### Synthesis of Oligonucleotides

Oligonucleotides of the invention may be produced by chemical synthesis using standard oligonucleotide synthesis methods known in the art. Methods may be purely synthetic, for example, the cyanoethyl phosphoramidite method (Beaucage & Caruthers, 1981, Tetrahedron Lett. 22: 1859-1862; McBride & Caruthers, Tetrahedron Lett. 24: 245-248). Synthetic methods useful for making oligonucleotides are also described by Ikuta et al. (1984, Ann. Rev. Biochem. 53: 323-356) (phosphotriester and phosphite-triester methods), and Narang et al. (1980, Methods Enzymol. 65: 610-620) (phosphotriester method). PNA molecules may be made using known methods such as those described by Nielsen et al. (1994, Bioconjug. Chem. 5: 3-7).

The H-phosphonate method for oligonucleotide synthesis may also be used. This method was first reported by Hale et al. (1957, J. Chem. Soc. 3291) and revisited later by Sekine and Hata (1975, Tetrahedron Lett. 16: 1711), Sekine et al. (1979, Tetrahedron Lett. 20: 1145), Garegg et al. (1985, Chemica Scripta 25: 280), and Garegg et al. (1986, Chemica Scripta 26: 59). The H-phosphonate method involves condensing the 5′ hydroxyl group of the nascent oligonucleotide with a nucleoside having a 3′ phosphonate moiety. Once the entire chain is constructed, the phosphite diester linkages are oxidized with t-butyl hydroperoxide or iodine to yield the corresponding phosphotriester. See, for example, Froehler (1993, “Oligodeoxynucleotide Synthesis”, Methods Mol. Biol. Vol. 20, Protocols for Oligonucleotides and Analogs, p. 63-80, S. Agrawal, Ed., Humana Press), and Uhlmann & Peyman (1990, Chem. Rev. 90: 543). The H-phosphonate method allows the production of modified backbones such as a phosphorothioate backbone (Stawinski & Strömberg, 2005, Methods Mol Biol. 288: 81-100) which is suitable for the present invention.

Since the oligonucleotides of this invention comprise different portions or regions, smaller subsequences of the oligonucleotides may be synthesised and then assembled by ligation (Borodina et al., 2003, Anal Biochem. 318(2): 309-313).

### Synthesis of Oligonucleotides Comprising Artificial Mismatch Discontinuities

Artificial mismatches may comprise natural nucleotides that are known to be non-complementary to the base at the appropriate position in the target sequence. Alternatively, universal nucleoside analogues may used such as 1-(2′-Deoxy-.beta.-D-ribofuranosyl)-3-nitropyrrole, which maximizes stacking interactions while minimizing hydrogen-bonding interactions without sterically disrupting a DNA duplex (Nichols et al., 1994, Nature 369: 492; and Bergstrom et al., 1995, J.A.C.S. 117: 1201). Similarly, the analogues 1-(2′-Deoxy-beta-D-ribofuranosyl)-5-nitroindole and 1-(2′-deoxy-beta-D-ribofuranosyl)-4-nitroimidazole may be used. However, nitropyrrole and nitroindole analogues are reported to be most favorable as universal nucleotides as they show the least discrimination for base pairing with natural nucleotides (Bergstrom et al., 1997, Nucleic Acids Res. 25(10): 1935-1942).

Abasic spacers may also be used such as the “D-spacer” in which deoxyribose residues without a base are introduced into the sequence of the probe oligonucleotide (see for example Takeshita et al., 1987, J Biol Chem. 262(21): 10171-10179).

The abasic spacer and the universal nucleotides are available as deoxyribose nucleotides but other sugar modifications, in particular 2′-modified sugars, are also envisaged.

### Synthesis of Oligonucleotides Comprising Non-Nucleic Acid Discontinuities

A 3-carbon spacer may be introduced into oligonucleotides as a discontinuity by employing a ‘C3 phosphoramidite spacer’. A C3 spacer will separate a pair of phosphodiester groups by approximately the same distance as ribose in terms of the backbone configuration.

Longer discontinuities may be introduced, for example as 9-atom, 12-atom and 18-atom spacers which are commercially available as phosphoramidites (for example from Glen Research Corporation, Sterling, Va., USA).

Several possibilities exist to introduce discontinuities in PNA oligonucleotides. Most FMOC amino acids may be used. Preferred spacers include glycine, alanine, beta-alanine, serine and lysine. Two alpha amino acids together will have approximately the same internucleotide distance as a single PNA nucleotide. Longer spacings can be introduced using more alpha amino acids or by introducing longer amino acids such as amino hexanoic acid.

### Nucleic Acid Analogues

The oligonucleotides of the invention may incorporate “artificial mismatches”, so the overall binding affinity of the oligonucleotides is reduced compared to unmodified probes. It may therefore be useful to use nucleic acid analogues with enhanced binding affinity compared to natural phosphodiester deoxyribosenucleic acids. It is known that RNA analogues with certain modifications at the 2′ position of the ribose ring show enhanced binding affinity for RNA targets compared to corresponding DNA/RNA hybrids (see Cummins et al., 1995, Nucleic Acids Res. 23(11): 2019-24). These RNA analogues also show reduced binding affinity for DNA compared to DNA/DNA hybrids (Tsourkas et al., 2003, Nucleic Acids Res. 31(6): 5168-74). The ability to bind preferentially to RNA over DNA with enhanced melting temperature makes 2′-modified analogues particularly useful for in situ hybridisation applications for detection of alternatively spliced RNA in a background of genomic DNA.

2′-O-methyl analogues in particular are readily available as phosphoramidite monomers for automated synthesis and are suitable for use with this invention. Additionally or alternatively, 2′-fluoro-modified analogues may be used.

Other nucleic acid analogues for use with this invention are ‘bridged’ analogues such as locked nucleic acids (“LNA”; Thomsen et al., 2005, RNA 11(11):1745-8) and 2′-4′-BNA(NC) (Rahman et al., 2008, J Am Chem Soc. 130(14): 4886-96). Bridged nucleic acid analogues show enhanced binding affinity for RNA compared with their natural nucleic acid counterparts, and are thus suitable for in situ hybridisation applications. Bridged analogues also show enhanced binding affinity for DNA compared with their natural nucleic acid counterparts, and are therefore useful for detection of chromosomal targets such as chromosomal translocations and for the detection of labelled cDNAs.

It is not normally desirable to synthesize oligonucleotides that are comprised entirely of bridged analogues. Hence LNA monomers are typically introduced every third base into DNA oligonucleotides (Válóczi et al., 2004, Nucleic Acids Res. 32(22): e175; Obernosterer et al., 2007, Nat Protoc. 2(6): 1508-14). LNA monomers may be introduced into 2’-O-methyl oligonucleotide sequences to enhance binding affinity of the resultant oligonucleotide (Kierzek et al., 2005, Nucleic Acids Res. 33(16): 5082-93). When LNA monomers are introduced into 2′-O-methyl oligonucleotides, the LNA monomers may be positioned every second base but in one embodiment not at the 5′ end of an oligonucleotide.

PNA is another analogue for use with this invention (Nielsen et al., 1994, Bioconjug Chem. 5(1): 3-7). PNA has enhanced binding affinity for both DNA and RNA targets compared to DNA oligonucleotides. PNA is less soluble than other DNA analogues and it is currently difficult to produce usable PNA oligonucleotides with a length greater than 20 bases. Hence PNA oligonucleotides may be shorter than oligonucleotides made with sugar/phosphate backbones. The invention encompasses oligonucleotides comprising lengths of PNA and DNA (see Uhlmann, 1998, Biol Chem. 379(8-9): 1045-52). These “mixed” oligonucleotides may be longer than PNA-only probes. Linkages between PNA and DNA can be useful points for introducing discontinuities in such mixed oligonucleotides.

### Oligonucleotide Characterisation

For several applications of the oligonucleotides, it may be useful to determine the Tₘ. Higher Tₘ values correspond to more stable duplexes. The stability of DNA duplexes can be calculated using known methods for prediction of melting temperatures (Breslauer et al., 1986, PNAS USA 83(11): 3746-3750; Lesnick & Freier, 1995, Biochemistry 34: 10807-10815, 1995; McGraw et al., 1990, Biotechniques 8: 674-678, 1990; and Rychlik et al., 1990, Nucleic Acids Res. 18: 6409-6412).

### Exemplar Oligonucleotides Designs

FIGS. 1a-c illustrate three different forms of oligonucleotides according to the invention. FIG. 1a shows an oligonucleotide comprising 2-O-methyl modified nucleotides. The oligonucleotide comprises two adjacent portions complementary in part to adjacent target nucleic acid domains. The two oligonucleotides portions are symmetrical about a portion junction (“PJ”; shown as a dashed line), which separates the portions in the oligonucleotide. The portion junction thus corresponds with a domain junction (for example, a splice junction or a gene fusion junction) in the target nucleic acid. Each of the oligonucleotide portions has a sequence of 21 bases. Starting from the portion junction, there are ten bases of 2-O-methyl sequence complementary to the first ten bases of the target nucleic acid domain adjacent the domain junction. The oligonucleotide sequence then comprises a discontinuity (at a position corresponding to base 11 of the target sequence downstream from the domain junction) formed from an abasic deoxyribose sugar residue. (In an alternative embodiment, ribose may be used instead of deoxyribose. Further alternatives are an abasic 2′-O-methylribose sugar or an abasic bridged sugar.) This is followed by a further ten bases of 2-O-methyl sequence complementary to the target nucleic sequence commencing at base 12 downstream from the domain junction. This portion structure is mirrored across the portion junction giving a total oligonucleotide length of 42 residues of which two are abasic residues acting as mismatches.

Different artificial mismatches could be inserted as discussed below with reference to further figures.

The discontinuity in the oligonucleotide shown in FIG. 1a is at a position corresponding to base 11 downstream from the domain junction in the target sequence but in this oligonucleotide and in other embodiments of the invention (for example, as described herein), the discontinuity could be located closer to the portion junction if desired. The overall length of the oligonucleotide can also be varied extensively while retaining enhanced specificity. In addition, two or more spacers could be introduced to provide a longer discontinuity. The flanking regions of complementary sequence would then start at a correspondingly increased distance from the domain junction.

FIG. 1b shows an oligonucleotide comprising 2-O-methyl modified nucleotides interspersed with LNA nucleotides. The oligonucleotide differs in structure from that shown in FIG. 1a in that the LNA nucleotides in FIG. 1b are positioned every second base, avoiding the 5′ end.

The oligonucleotide in FIG. 1b is symmetrical about the portion junction and as such the alternating pattern of LNA insertions is not continued across the junction (which is flanked instead of two adjacent 2-O-methyl nucleotides). In an alternative symmetric Tₘ design, continuous runs of alternating 2′-O-methyl and LNA nucleotides may be used, for example an oligonucleotide having the sequence: 5′-(OMe-LNA)₅-dSpacer-(OMe-LNA)₁₀-dSpacer-(OMe-LNA)₅-3′.

FIG. 1c shows an oligonucleotide comprising DNA nucleotides interspersed with LNA nucleotides. The oligonucleotide differs in structure to that shown in FIG. 1b in that LNA nucleotides are positioned every third base in FIG. 1c.

As an alternative to the example shown in FIG. 1c, LNA nucleotides may be positioned differently in the oligonucleotide sequence. An alternative oligonucleotide design may for example have the following structure: 5′-LNA-(DNA-DNA-LNA)₃-dSpacer-(DNA-DNA-LNA)₆-dSpacer-(LNA-DNA-DNA)₃-LNA-3′.

FIGS. 2a-b illustrate two further forms of oligonucleotides according to the invention. FIG. 2a shows an oligonucleotide comprising 2-O-methyl modified nucleotides. The oligonucleotide differs in structure from that shown in FIG. 1a in that the discontinuities in the oligonucleotide of FIG. 2a are artificial mismatches formed by the universal base nitropyrrole. In variations of this oligonucleotide, universal nucleotides such as nitroindole or nitroimidazole could be used to create artificial mismatches.

FIG. 2b shows an oligonucleotide comprising 2-O-methyl modified nucleotides interspersed with LNA nucleotides. The oligonucleotide differs in structure from that shown in FIG. 1b in that the discontinuities in FIG. 2b comprise artificial mismatches using the universal base nitroindole. In variations of this oligonucleotide, universal nucleotides such as nitropyrrole or nitroimidazole could be used to create artificial mismatches.

FIGS. 3a-c illustrate three further forms of oligonucleotides according to the invention. FIG. 3a shows an oligonucleotide comprising 2-O-methyl modified nucleotides. The oligonucleotide differs in structure from that shown in FIG. 1a in that the discontinuity as shown in FIG. 3a comprises a 3-carbon spacer (C3 spacer).

FIG. 3b shows an oligonucleotide comprising 2-O-methyl modified nucleotides interspersed with LNA nucleotides. The oligonucleotide differs in structure from that shown in FIG. 1b in that the discontinuity as shown in FIG. 3b comprises a 12-atom ethylene glycol linker. The 12-atom ethylene glycol linker is fairly flexible, so the second ten bases of sequence in each portion of the oligonucleotide could be complementary to target domains further from the domain junction than in the FIG. 1b oligonucleotide. For example, base 12 of the oligonucleotide shown in FIG. 3b may be complementary to base 13 or 14 from the domain junction in the target sequence. For the oligonucleotide shown in more generally according to the invention, more than one spacer may be used if a larger interruption in the continuity of the oligonucleotide sequence with respect to the target sequence is required. Other spacers such as a C3 spacer may additionally be incorporated.

As with the probe,shown in FIG. 1b, the LNA nucleotides in the FIG. 3b oligonucleotide are inserted every second base, avoiding the 5′ terminus. In an alternative symmetric Tₘ design, continuous runs of alternating 2′-O-methyl and LNA nucleotides may also be used, for example an oligonucleotide having the sequence: 5′-(OMe-LNA)₅-Linker-(OMe-LNA)₁₀-Linker-(OMe-LNA)₅-3′.

FIG. 3c shows an oligonucleotide comprising DNA nucleotides interspersed with LNA nucleotides. The oligonucleotide differs in structure from the oligonucleotide shown in FIG. 1c in that the discontinuity as shown in FIG. 3c is an 18-atom ethylene glycol linker. The second ten bases of sequence in each portion of the oligonucleotide could be complementary to target sequences further from the portion junction than in the FIG. 1c oligonucleotide, if a greater interruption in the continuity of the oligonucleotide sequence with respect to the target sequence is required. The alternative design described with respect to FIG. 1c is also suitable when using an 18-atom ethylene glycol linker to produce a mismatch.

FIG. 4 illustrates a PNA-containing oligonucleotide according to the invention. As for the sugar/phosphate oligonucleotide shown in FIGS. 1-3, the PNA probe comprises two portions of sequence complementary to adjacent target nucleic acid domains (such as domains adjacent a splice junction or a gene fusion junction). The two portions are shown on either side of a dashed line marking the portion junction which separates the junctions in the oligonucleotide. The dashed line accordingly corresponds with the domain junction which separates domains in the target. Each of the junction sequences consist of eight bases of sequence. Starting from the portion junction, there are three bases of PNA sequence in the oligonucleotide complementary to the first three bases in the target adjacent the domain junction. Then there is a discontinuity comprising two glycine residues (a spacer equivalent to one PNA residue). This is followed by a further four bases of PNA sequence complementary to the target sequence that starts five bases downstream from the domain junction. This structure is mirrored across the portion junction giving a total oligonucleotide length of sixteen residues of which two are spacer glycine residues acting as discontinuities.

The oligonucleotides in FIGS. 1-4 are shown without labels. However, labels may be incorporated into each of the oligonucleotide, for example at locations as discussed elsewhere.

### Base Modifications

FIGS. 5(i)-(viii) illustrate various bases that can be incorporated into the oligonucleotides of the invention. FIGS. 5(i) to 5(iv) illustrate the standard “Watson-Crick” bases adenine, thymine, cytosine and guanine, respectively. Several modifications may be made to these standard bases. Such modifications may normalise or increase base pairing energies. Non-limiting examples of modified bases are shown in FIGS. 5(v)-5(viii).

FIG. 5(v) shows a cytosine analogue 5-propynyl cytosine which pairs more stably with guanine than standard cytosine (Wagner et al., 1993, Science. 260(5113): 1510-3).

FIG. 5(vi) shows a thymine analogue 5-propynyl uracil which pairs more stably with adenine than standard thymine.

FIG. 5(vii) shows an adenine analogue 2,6-diaminopurine which forms three hydrogen bonds with thymine, rather than two formed with standard adenine. The analogue therefore pairs more stably with thymine than standard adenine (Gaffney et al., 1984, Tetrahedron 40: 3-13).

FIG. 5(viii) shows a cytosine analogue 5-methyl cytosine which pairs more stably with guanine than standard cytosine. 5-methyl cytosine base substitutions when combined with 2′-O-methoxyethyl sugar modifications are particularly stable (see U.S. Pat. No. 6,503,754 and U.S. Pat. No. 6,506,735).

Other examples of modified bases for use in the invention include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-fluoro-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine (1H-pyrimido[5,4-b][1,4]benzoxazin-2(3H)-one), phenothiazine cytidine (1-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further bases include those disclosed in: U.S. Pat. No. 3,687,808; The Concise Encyclopedia Of Polymer Science And Engineering, 1990, pages 858-859, Kroschwitz (ed.), John Wiley & Sons; Englisch et al., 1991, Angewandte Chemie, International Edition 30: 613; and Sanghvi, 1993, Antisense Research and Applications, Chapter 15, pages 289-302, Crooke & Lebleu (eds), CRC Press. Certain of these bases are particularly useful for increasing the binding affinity of the oligonucleotides of the invention. These include 5-substituted pyrimidines, 6-azapyrimidines and N-2, N-6 and 0-6 substituted purines, including 2-aminopropyladenine.

These and other base modifications known in the art make it possible for example to alter the temperature range at which oligonucleotides can hybridise specifically to their target nucleic acid.

### Backbone Modifications

Oligonucleotides may be readily modified in the phosphate moiety. Under certain conditions, such as low salt concentration, analogues such as methylphosphonates, (Miller et al., 1983, Nucleic Acids Res. 11(18): 6225-42), triesters (Miller et al., 1974, Biochemistry 13(24): 4887-96) and phosphoramidates have been shown to increase duplex stability. A known method for synthesis of methylphosphonates produces a mixture of stereoisomers with the methylated phosphate groups acting as chiral centres. It has been shown that if chirally pure methylphosphonates adopt the R-configuration at every methylphosphonates centre, the resulting oligonucleotide has relatively high binding affinity to biological nucleic acids even at physiological salt concentrations (Vyazovkina et al., 1994, Nucleic Acids Res. 22(12): 2404-9). Similar results have been shown for phosphoramidate backbones, where the amide replaces a non-linking oxygen (Kitamura et al., 1990, Nucleic Acids Symp Ser. (22): 65-6). Methylphosphonates, phosphoramidates and triesters also have increased nuclease resistance. So-called N3→P5′ phosphoramidates are also advantageous as they have a neutral backbone and are simple to produce in a specific stereochemical conformation (Gryaznov et al., 1995, PNAS USA 92(13): 5798-802).

Further phosphate modifications include phosphorothioates (Efimov et al., 1995, Nucleic Acids Res. 23(20): 4029-33), phosphorodithioates (Capaldi et al., 2000, Nucleic Acids Res. 28(9): E40) and boranophosphates (Shimizu et al., 2006, J Org Chem. 71(11): 4262-9), each of which increase the resistance of oligonucleotides to exonuclease degradation. Isosteric replacement of phosphorus by sulphur gives nuclease resistant oligonucleotides (Huang & Benner, 2002, J Org Chem. 67(12): 3996-4013). Replacement by carbon at either phosphorus or linking oxygen is also a further possibility (Milligan et al., 1993, J. Med. Chem. 36(14): 1923-1937).

The backbone modifications discussed above are of particular usefulness when the oligonucleotides of the invention are for use in tissue hybridisation or as antisense therapeutics.

### Mass Tagged Oligonucleotides

A variety of mass tags can be used with this invention. For example, suitable mass tags are disclosed in WO 97/27327, WO 97/27325, WO 97/27331 and WO 03/025576. These publications disclose tags that comprise polyamide compounds, essentially peptides or peptide-like tags, which means that these tags can be prepared using a number of peptide synthesis methods that are well known in the art (see for example Jones, 1991, “The chemical synthesis of peptides”, Oxford University Press; Fields & Noble, 1990, Int J Pept Protein Res 35(3): 161-214; Albericio, 2000, Biopolymers 55(2):123-139). In addition, the use of peptide and peptide-like tags enables coupling of these tags to oligonucleotides using a variety of peptide conjugation techniques that are known in the art. Methods for coupling peptides to oligonucleotides “on column” via 5′ amine functionalities are disclosed in Zaramella et al. (2004, J Am Chem Soc 126(43): 14029-14035). Methods for conjugating peptides to oligonucleotides via thiol groups at the termini of the oligonucleotides are disclosed in Arar et al. (1995, Bioconjug Chem. 6(5): 573-577). Oligonucleotides can be coupled to peptides with terminal cysteine residues as disclosed in Wei et al. (1994, Bioconjug Chem. 5(5): 468-74).

In one approach, peptide tags can be synthesised on Controlled Pore Glass (CPG) beads of the kind used for DNA synthesis. As long as these peptides are suitably protected and as long as the peptide has a free hydroxyl (or trityl protected hydroxyl) at the end of the peptide synthesis, an oligonucleotide can be synthesized directly on the peptide (Haralambidis et al., 1990, Nucleic Acids Res. 18(3): 493-499). The peptide should be linked to the resin with a suitable linker that will be cleaved under conditions normally used for cleaving and deprotecting oligonucleotides. A 4-Hydroxymethylbenzoic acid (HMBA) linker (Sheppard et al., 1982, Int. J. Peptide Protein Res. 20: 451) is base-cleavable and suitable for both FMOC peptide synthesis and oligonucleotide synthesis. This can be readily coupled to amino-functionalized CPG beads such as aminopropyl-CPG, which is commercially available.

An example of a synthesis according to this embodiment of the invention is illustrated in FIG. 6. In FIG. 6 it can be seen that tagged oligonucleotide conjugates according to the invention are prepared by initially synthesizing the peptide tag component of the conjugate on a CPG resin that is compatible with automated oligonucleotide synthesis. In step (1) of the method in FIG. 6, aminopropyl CPG is initially coupled to the aforementioned HMBA linker. Peptide synthesis is initiated from this linker. In step (2), an O-trityl protected FMOC serine residue is coupled to the HMBA linker. Spacers such as beta-alanine could be introduced between the serine and the HMBA linker if desired to reduce potential steric interference from the resin. In step (3), multiple cycles of standard FMOC synthesis takes place, i.e. the desired peptide tag is synthesised using commercially available FMOC-amino acids. In this example, an FMOC protected photocleavable linker (4-[4-(1-(FMOC-amino)ethyl)-2-methoxy-5-nitrophenoxy)butanoic acid available from IRIS Biotech GmbH, Marktredwitz, Germany) is introduced followed by FMOC-alanine, followed by 1-Fmoc-piperidin-4-ylacetic acid (Sigma Aldrich, UK), followed again by FMOC-alanine, which is finally acetylated to block the terminal amino group so that it does not interfere in subsequent oligonucleotide synthesis. Between step (3) and step (4), the CPG resin is transferred from the automated peptide synthesizer reaction vessel or column into a column or vessel suitable for use in an automated DNA synthesizer. In step (4), the trityl protection group is removed using standard deprotection conditions in the DNA synthesizer, typically trichloroacetic acid or dichloroacetic acid in dichloromethane. In step (5), an optional Dimethoxtrityl (DMTr) protected linker (or “spacer”) is introduced to reduce steric hindrance from the solid support and the peptide (Spacer Phosphoramidite 9 from Glen Research, Stirling, Va., USA). A wide variety of similar spacers is known in the art and could be substituted for the linker shown, if desired. In step (6), multiple cycles of standard automated phosphoramidite DNA synthesis are carried out to generate the desired DNA sequence of the oligonucleotide, linked to the peptide. In the final step (7), the side chain deprotection groups are cleaved along with the HMBA linker releasing the deprotected conjugate into solution (also referred to as an NH₃/H₂O step). Typically, the released conjugate is then purified by high performance liquid chromatography (HPLC), gel filtration, gel electrophoresis or other standard purification techniques known in the art.

The method shown in FIG. 6 introduces the peptide tag at the 3′ end of the oligonucleotide. However, “reverse’ synthesis” (5′ to 3′) is also possible (see Claeboe et al., 2003, Nucleic Acids Res. 31(19): 5685-91). Reverse synthesis phosphoramidites used with the method in FIG. 6 (and also the methods in FIGS. 7 and 8) would yield oligonucleotides modified at the 5′ terminus. Phosphoramidite monomers for 5′ to 3′ synthesis are commercially available, for example from Glen Research Corporation (Sterling, Va., USA).

Many amino acids are known in the art and not all are available as FMOC derivatives. It is, however, possible for one of ordinary skill in the art to prepare FMOC protected derivatives of unprotected amino acids for use with the present invention using standard methods (for example, Fields & Noble, 1990, Int J Pept Protein Res. 35(3): 161-214).

PNA peptide conjugates can be readily synthesized as both PNA and peptides can be synthesized by FMOC chemistry. Examples of such oligonucleotides are disclosed in Thompson et al. (2007, Nucleic Acids Res. 35(4): e28).

To allow more than one tag to be incorporated per oligonucleotide, mass tags can be incorporated into the oligonucleotide through conjugation to thymidine analogues, for example, as disclosed in Brown et al. (2001, Tetrahedron Lett. 42: 2587-2592). In that publication, a thymidine analogue is described with a linker coupled to the purine ring of the thymidine. This thymidine analogue has a hydroxyl group protected with an FMOC group on the end of the linker that can be made available after the nucleotide has been coupled into an oligonucleotide during automated oligonucleotide synthesis to allow a phosphoramidite modified tag to be incorporated into an oligonucleotide. Since this analogue can be incorporated within the chain, multiple linkers and hence tags can be couple to the oligonucleotide.

Alternatively, branched peptides can be synthesized incorporating multiple tag peptide sequences. The branched peptides can be synthesized on CPG, with subsequent synthesis of the oligonucleotide (see Haralambidis et al., 1990, Nucleic Acids Res. 18(3): 501-505).

Examples of schemes according to this embodiment of the invention are shown in FIGS. 7 and 8. In FIG. 7, it can be seen that tag oligonucleotide conjugates according to this invention are prepared by initially synthesizing the peptide tag component of the conjugate on a controlled pore glass (CPG) resin that is compatible with automated oligonucleotide synthesis. In step (1) of FIG. 7, aminopropyl CPG is initially coupled to the aforementioned HMBA linker. A spacer could be introduced between the aminopropyl glass and the linker of desired or a CPG resin that has a longer linker could be employed. Peptide synthesis is initiated from the HMBA linker. In step (2) of the method in FIG. 7, an N-alpha-Fmoc-N-epsilon-1-(4,4-dimethyl-2,6-dioxocyclohex-1-ylidene)ethyl-L-lysine (α-FMOC-ε-Dde-Lys) residue is coupled to the HMBA linker. One or more spacers such as beta-alanine could be introduced between the lysine and the HMBA linker if desired to reduce potential steric interference from the resin. The Dde protection group is removable under 2% hydrazine in Dimethylformamide (DMF) (Bycroft et al., 1993, J. Chem. Soc., Chem. Commun. 778-779) and is used for the purpose of synthesizing branched peptides. In step (3), a further α-FMOC-ε-Dde-Lys residue is coupled, followed by an α-FMOC-O-trityl protected serine residue. The final FMOC group is removed and the serine amino group is acetylated to prevent further reaction. In step (4), the Dde protection groups are removed. In step (5), multiple cycles of FMOC peptide synthesis are conducted, i.e. the desired peptide tag is synthesized from both of the free epsilon amino groups exposed by the removal of the Dde groups using commercially available FMOC-amino acids. In this example, an FMOC protected photocleavable linker (4-[4-(1-(FMOCamino)ethyl)-2-methoxy-5-nitrophenoxy)butanoic acid available from IRIS Biotech GmbH, Marktredwitz, Germany) is introduced followed by FMOC-alanine, followed by 1-Fmoc-piperidin-4-ylacetic acid (Sigma Aldrich, UK), followed again by FMOC-alanine, which is finally acetylated to block the terminal amino group so that it does not interfere in subsequent oligonucleotide synthesis. Between step (5) and step (6), the CPG resin is transferred from the automated peptide synthesizer reaction vessel or column into a column or vessel suitable for use in an automated DNA synthesizer. In step (6) of FIG. 7, the trityl protection group is removed using standard deprotection conditions in the DNA synthesizer, typically trichloroacetic acid or dichloroacetic acid in dichloromethane. In step (7), an optional Dimethoxtrityl (DMTr) protected linker (or “spacer”) is introduced to reduce steric hindrance from the solid support and the peptide (here, Spacer Phosphoramidite 9 from Glen Research, Stirling, Va., USA). A wide variety of similar spacers is known in the art and could be substituted for the linker shown, if desired. In step (8), multiple cycles of standard automated DNA synthesis are carried out to generate the desired DNA sequence linked to the peptide. In the final step (9, the side chain deprotection groups are cleaved along with the HMBA linker releasing the deprotected conjugate into solution (also referred to as an NH₃/H₂O step). Typically, the released conjugate would then be purified by high performance liquid chromatography, gel filtration, gel electrophoresis or other standard techniques known in the art.

The number of peptides linked to a single oligonucleotide can be varied by varying the number of α-FMOC-ε-Dde-Lysine residues introduced into the peptide in the first steps of the synthesis shown in FIG. 7. If only a single tag is desired then only one α-FMOC-ε-Dde-Lysine residue need be coupled, but if 5 tags were desired this can be achieved by coupling 5 α-FMOC-ε-Dde-Lysine residues.

In principle, the order of certain steps can be varied as well. The introduction of the optional spacer in step (7) of FIG. 7 could take place prior to removal of the Dde groups. This may be desirable if many peptides or if large peptides are to be coupled to a single oligonucleotide causing steric hindrance that might reduce the efficiency of the introduction the linker.

In further variations on the method in FIG. 7, α-Dde-ε-FMOC-Lysine can be employed in place of α-FMOC-ε-Dde-Lysine. In one variation, the O-trityl serine needed to initiate oligonucleotide synthesis can be coupled at the epsilon amino while the peptide tag is synthesized at the alpha position after removal of the Dde group. Alternatively, the alpha-Dde group can be removed prior to the epsilon-FMOC group, and the O-trityl serine can be coupled at this position. One or more optional spacers, such as beta-alanine residues can be introduced before coupling of the O-trityl protected serine, if desired.

In the example shown in FIG. 8, it can be seen that tag oligonucleotide conjugates according to a further embodiment of the present invention are prepared by initially synthesizing the peptide tag component of the conjugate on a CPG resin that is compatible with automated oligonucleotide synthesis. In step (1) of FIG. 8, aminopropyl CPG is initially coupled to an HMBA linker. Peptide synthesis is initiated from this linker. In step (2), an α-FMOC-O-trityl protected serine residue is coupled to the HMBA linker. One or more spacers such as beta-alanine could be introduced between the serine residue and the HMBA linker if desired to reduce potential steric interference from the resin. In step (3), an α-FMOC-ε-FMOC-protected Lysine residue is introduced and coupled to the serine residue. In step (4), multiple cycles of standard FMOC peptide synthesis are conducted, i.e. the desired peptide tag is synthesized from both of the free amino groups exposed by the removal of the FMOC groups on the lysine amino groups. The peptide tags are synthesized using commercially available FMOC-amino acids. In this example, an FMOC protected photocleavable linker (4-[4-(1-(FMOC-amino)ethyl)-2-methoxy-5-nitrophenoxy)butanoic acid available from IRIS Biotech GmbH, Marktredwitz, Germany) is introduced followed by FMOC-alanine, followed by 1-Fmoc-piperidin-4-ylacetic acid (Sigma Aldrich, UK), followed again by FMOC-alanine, which is finally acetylated to block the terminal amino group so that it does not interfere in subsequent oligonucleotide synthesis. Between step (4) and step (5), the CPG resin is transferred from the automated peptide synthesizer reaction vessel or column into a column or vessel suitable for use in an automated DNA synthesizer. In step (5), the trityl protection group is removed using standard deprotection conditions in the DNA synthesizer, typically trichloroacetic acid or dichloroacetic acid in dichloromethane. In step (6), an optional Dimethoxtrityl protected linker (or “spacer”) is introduced to reduce steric hindrance from the solid support and the peptide (Spacer Phosphoramidite 9 from Glen Research, Stirling, Va., USA). A wide variety of similar spacers is known in the art and could be substituted for the linker shown, if desired. In step (7), multiple cycles of standard automated DNA synthesis are carried out to generate the desired DNA sequence linked to the peptide. In the final step (8), the side chain deprotection groups are cleaved along with the HMBA linker releasing the deprotected conjugate into solution (also referred to as an NH₃/H₂O step).

Typically, the released conjugate would then be purified by HPLC, gel filtration, gel electrophoresis or other standard purification techniques known in the art. The number of peptides linked to a single oligonucleotide can be varied by varying the number of α-FMOC-ε-FMOC-Lysine residues introduced into the peptide in the first steps of the synthesis. After the introduction of the first α-FMOC-ε-FMOC-Lysine, removal of the FMOC groups will expose two amino groups. This means in the next cycle of synthesis 2 further α-FMOC-ε-FMOC-Lysine groups could be introduced and four in the next cycle if desired. Three cycles of coupling of α-FMOC-ε-FMOC-Lysine would make 8 amino groups available for the synthesis of peptide tags.

Hybrid methods employing the strategies of FIGS. 6-9 are envisaged. For example, steps (1) to (4) of FIG. 6 could be completed, i.e. up to the removal of the Dde groups, then α-FMOC-ε-FMOC lysine residues could be coupled to the amino groups exposed by removal of the Dde groups allowing further branching to be introduced if desired.

Several photocleavable linkers for use in the invention are commercially available. For example, the FMOC protected linker 4-[4-(1-(FMOCamino)ethyl)-2-methoxy-5-nitrophenoxy)butanoic acid is commercially available from Iris Biotech GmbH (Marktredwitz, Germany) and from Advanced ChemTech, Inc (Kentucky, USA). This can be readily incorporated into peptides during conventional FMOC synthesis.

Branched peptides (or other polyamides) with one or more attached oligonucleotides, and comprising a plurality of cleavable branches, form another aspect of the invention.

Non-natural amino acids may be used in this invention, including FMOC-Piperazin-1-ylacetate, 1-Fmoc-piperidin-4-ylacetic acid, N,N-Dimethyl glycine, β-Dimethylamino-DL-alanine (all commercially available from Sigma Aldrich, UK).

In addition to the tags shown in FIGS. 7 and 8, exemplar peptide sequences of the invention include:

Series 1:

Alanine—Piperazin-1-ylacetic acid—Alanine

Alanine—Piperazin-1-ylacetic acid—Valine

Valine—Piperazin-1-ylacetic acid—Alanine

Valine—Piperazin-1-ylacetic acid—Valine;

Series 2:

N,N-Dimethyl glycine—Alanine—Proline—Alanine [SEQ ID NO: 1]

N,N-Dimethyl glycine—Alanine—Proline—Valine [SEQ ID NO: 2]

N,N-Dimethyl glycine—Valine—Proline—Alanine [SEQ ID NO: 3]

N,N-Dimethyl glycine—Valine—Proline—Valine [SEQ ID NO: 4]; and

Series 3:

Cysteic Acid—Alanine—Aspartic acid—Alanine [SEQ ID NO: 5]

Cysteic Acid—Alanine—Aspartic acid—Valine [SEQ ID NO: 6]

Cysteic Acid—Valine—Aspartic acid—Alanine [SEQ ID NO: 7]

Cysteic Acid—Valine—Aspartic acid—Valine [SEQ ID NO: 8].

In the series 1 peptides, it can be seen that the collision cleavable linker (Piperazin-1-ylacetic acid) is also a charge-carrying group due to the presence of the tertiary amino group in the piperazine ring. In series 2 and 3 peptides, the charge-carrying groups are separate entities, i.e. N,N-Dimethyl glycine and cysteic acid, respectively. In alternative embodiments, the charge-carrying groups could act as the mass modifier as well, allowing the adjacent mass modifier amino acid group to be removed resulting in a smaller tag if that were desirable.

In the series 2 peptides, the proline residue can be substituted with piperidin-4-ylacetic acid. In series 2, the N,N-Dimethyl glycine can be replaced with any easily protonated positive charge-carrying group including the natural amino acids lysine and histidine, tertiary amino group containing molecules such as β-Dimethylamino-alanine. Secondary amino containing groups may also be introduced such as nipecotic acid. Pyridine containing compounds such as nicotinic acid are also appropriate.

FIG. 9a illustrates a mass tagged oligonucleotide probe based on the peptide (N)—Acetate—Valine—Piperazin-1-ylacetic acid—Alanine—Beta-alanine—(C) and its tag product following photocleavage (“P”). This structure is another variant of the series-1 peptides shown above. FIG. 9b illustrates a set of 9 isobaric tags constructed by combinatorial rearrangement of the mass modifier amino acids, beta-alanine, alanine and valine, and isotopes of acetic acid in the tags. All of the isotope-substituted reagents are commercially available (for example, from Cambridge Isotope Laboratories, Inc; Andover, Mass., USA).

Large numbers of different sets of tags can be constructed by changing the amino acids and other entities used as mass modifiers. The acetate group blocking the N-terminal amino group of the peptide tags could be replaced with any carboxylic acid, e.g. propionic or butyric acids, which can be obtained with isotope substitutions (for example, from Cambridge Isotope Laboratories, Inc; Andover, Mass., USA). Similarly, further isobaric compounds can be obtained by swapping the positions of the alanine and valine residues, for example. Synthesis of every combination of isotope substituted amino acids shown in this sequence would give rise to hundreds of different tags, although not all would be isobaric with each other. If every possible, isotopic variant of the sequence shown in FIG. 9b was synthesized, the tags would fall into a series of isobaric sets. Although these variants are too numerous to enumerate here, they are encompassed by the present invention.

In all these series the mass modifiers comprise individual amino acids of valine or alanine or their isotopes. However, other amino acids can be used for this purpose. In addition, more than one amino acid can be inserted into the sequence to act as mass modifiers. To ensure cleavage is most favoured at a particular site in the sequence, beta-amino acids can also be used to act as mass modifiers as these do not readily support formation of oxazolone structures and thus do not cleave as easily as alpha amino acids at any given collision energy. In FIG. 9b the combination of alanine and beta-alanine is used to act as a mass modifier. Similarly, the combination of valine and acetate is also used to act as a single mass modifier in FIG. 9b.

In an alternative embodiment, an acid cleavable linker can be used. Since most MALDI matrix materials are acidic, addition of the matrix will effect cleavage of the mass tags. A simple method for introducing an acid labile group is to include a P3′-N5′ phosphoramidate at the 5′ terminus of the oligonucleotide adjacent to the mass tag (Shchepinov et al., 2001, Nucleic Acids Res. 29(18): 3864-72).

In a further embodiment, the entire probe label complex can be desorbed, and cleavage of the tags can take place by collision using Post Source Decay in a Time-Of-Flight mass spectrometer or in the mass analyzer of an ion trap instrument or in a collision cell in alternative geometries that are used with MALDI, such as the Q-TOF geometry. Suitable PNA probes with a collision cleavable linker between the tag peptide and a PNA probe have been described previously (see Thompson et al., 2007, Nucleic Acids Res. 35(4): e28).

Other useful mass tags for use with this invention include electrophores (Britt et al., 1996, J Mass Spectrom. 31(6): 661-8; Xu et al., 1997, Anal Chem. 69(17): 3595-602; Zhang et al., 2002, Bioconjug Chem. 13(5): 1002-12).

### Fluorescently Tagged Oligonucleotides

A variety of fluorescent tags suitable for labelling the oligonucleotides of the invention are known in the art. Fluorescein is widely used as a fluorescent reagent for oligonucleotide labelling. In addition, related compounds including rhodamine and its derivatives, eosin and its derivatives are all useful. Methods for introducing these kinds of dye into oligonucleotides are well known (see Urdea et al., 1998, Nucleic Acids Res. 16(11): 4937-56; Giusti & Adriano, 1993, PCR Methods Appl. 2(3): 223-7). Cyanine dyes are also suitable labels for oligonucleotides of the present invention (Anderson et al., 1993, “Synthesis of a Carbocyanine Phosphoramidite and its use in Oligonucleotide Labelling”, International Conference on Nucleic Acid Medical Applications; Southwick et al., 1990, Cytometry 11: 418-430). Moreover, Alexa dyes are another suitable class of dyes for labelling of oligonucleotides (see Panchuk-Voloshina et al., 1999, J Histochem Cytochem. 47(9): 1179-88).

Binding of unlabelled oligonucleotides to unlabelled targets may be detected using intercalating dyes. Exemplary dyes include SYBR Green, Thiazole Orange, TOTO (a dimer of Thiazole Orange), Oxazole Yellow (YO and its dimer YOYO) (Rye et al., 1992, Nucleic Acids Res. 20(11): 2803-12; Schneeberger et al., 1995, PCR Methods Appl. 4(4): 234-8.

### Immobilisation of Oligonucleotides

Suitable methods for immobilization of oligonucleotides to solid-phase supports are well known in the art. For example, suitable attachment methods are described by Pease et al. (1994, PNAS USA 91(11): 5022-5026) and Khrapko et al. (1991, Mol Biol (Mosk) (USSR) 25: 718-730). A method for immobilization of 3′-amine oligonucleotides on casein-coated slides is described by Stimpson et al. (1995, PNAS USA 92: 6379-6383). Particularly useful methods of attaching oligonucleotides to solid-state substrates are described by Maskos and Southern (1992, Nucleic Acids Res. 20(7): 1679-1684) and Guo et al. (1994, Nucleic Acids Res. 22: 5456-5465).

### Microarray Construction

In certain embodiments of the invention, modified oligonucleotides are provided as arrays of oligonucleotides on a planar substrate, where each distinct oligonucleotide is present at a distinct location on the planar array. These arrays may also be referred to as microarrays. There are two main methods of manufacturing oligonucleotide arrays: in situ synthesis and robotic spotting of pre-synthesised oligonucleotides (for examples, see Yershov et al., 1996, PNAS USA 93: 4913-4918). In situ synthesis has the advantage, in principle, of low cost as the array is assembled directly from small quantities of the 4-nucleotide monomers. The manufacturing process is highly automated and precise. A disadvantage of the in situ approach, however, is that it is not possible to purify the oligonucleotide located in each spot on the array.

The first in situ synthesised arrays were produced in the laboratories of Ed Southern using a glass “mask” etched with microfabricated fluidic channels linked to a conventional oligonucleotide synthesiser. The mask plate is placed on top of a suitably derivatised glass array surface, such as a microscope slide. The synthesiser then delivers reagents to each channel in the required order to perform conventional oligonucleotide synthesis on the microscope slide surface. In this way, narrow strips of oligonucleotides can be synthesised on a microscope slide.

A second technology uses a circular delivery device linked to an oligonucleotide synthesiser. The delivery device is clamped to the surface of prepared microarray slide. The desired nucleotide monomer is delivered to the array surface. The delivery device is then detached and moved along the surface of the slide so that it still largely overlaps the area to which the first monomer has been delivered, and then the second desired monomer is delivered. This extends the first monomer into a dinucleotide in the region that overlaps with the first delivery area, while adding a single monomer to the area that has not yet been treated. The delivery device is then shifted to a third partially overlapping location where the third monomer is delivered. The process is repeated until the entire desired sequence of a gene is delivered to the surface of the slide. The end result of this sequential synthesis is that the entire sequence of a gene is synthesised on a glass slide in overlapping sequences of a pre-determined length (for references see: Maskos & Southern, 1993, Nucleic Acids Res. 21: 2269-2270; Southern et al, 1994, Nucleic Acids Res. 22: 1368-1373). This approach uses normal phosphoramidates but commercially available universal nucleotides and spacers discussed above could be employed directly with the Southern approach to array construction, allowing arrays of oligonucleotides according to the present invention to be synthesized.

A further approach to in situ manufacture of short oligonucleotides (11-25-mer) employs photolithography. Specialist phosphoramidate monomers with photo-cleavable protective groups are required for this technology and specialist mask illumination equipment. The photolithographic method for in situ array synthesis starts with a reactive surface that has been protected with the photo-labile masking groups. Regions of the surface designated to be reacted with a particular nucleotide monomer are activated by exposing that region with light to remove the photo-labile protective group. In practice, this is effected by shining light through a “mask” so that only desired regions of the array surface are exposed to light. The cleaved protected groups are washed away and the desired protected nucleotide monomer is coupled to the exposed surface. The coupling reaction effectively blocks all the available reactive groups with protected nucleotide monomer. The next designated base can then be added to the array by exposing the required areas with light. If a region that has already been coupled to a nucleotide is exposed then the protective groups are removed from the protected nucleotide making it available for chain extension. Addition of the next protected nucleotide will then either be coupled to the exposed surface of the array or to unprotected nucleotide. Thus after the second cycle of deprotection and coupling the surface will comprise unreacted regions, regions with a single nucleotide attached and regions with dinucleotides attached. The process can be continued until the surface of the array is derivatised at each location with a specific desired nucleotide. To produce an array of N-mers should require no more than 4×N deprotection and coupling steps, and in general it takes fewer steps (for reference see Pease et al., 1994, PNAS USA 91: 5022-5026; Nuwaysir et al., 2002, Genome Res. 12(11): 1749-55).

For the purposes of the present invention, arrays employing the above photolithographic methods require spacer and universal nucleotide analogues that have photocleavable protective groups. A number of other approaches to in situ photolithographic synthesis of oligonucleotide arrays have also been developed that do not require specialist phosphoramidates, and would thus allow arrays of oligonucleotides according to this invention to be constructed using photolithographic methods and conventional nucleotides (see McGall et al., 1996, PNAS USA 93(24): 13555-60; Serafinowski et al., 2003, J Am Chem Soc. 125(4): 962-5).

A further suitable method of in situ oligonucleotide synthesis uses inkjet dispensing to deliver standard phosphoramidates to specific locations on a microarray slide in the required order to produce the desired sequences (Hughes et al., 2001, Nat Biotechnol. 19: 342-347). That method uses conventional oligonucleotide synthesis reagents and protocols, relying on accurate dispensing to deliver the desired reagents to the correct locations on the array.

For the purposes of the present invention, arrays of in situ synthesized modified oligonucleotides according to this invention may be constructed using any of the above in situ oligonucleotide synthesis methods.

Although in situ synthesis has many attractions, it requires specialist equipment. In contrast, robotic spotting uses widely available robotic equipment. In the robotic spotting approach, pins or “quills” are dipped by a robotic arm or translation stage, into microtitre plates with different oligonucleotides in each well and delivered to desired locations on a microscope slide. All that is required are microtitre plates with the desired oligonucleotides. An advantage of robotically spotted oligonucleotides is the pre-synthesis of oligonucleotides. This allows purification of the oligonucleotides and validation of the sequence before use, which is useful for the present invention particularly when oligonucleotides of 50 nucleotides or more in length are used.

Coupling of oligonucleotides to an array surface may involve the synthesis of amine- or thiol-modified oligonucleotides. These nucleophilic functional groups can covalently react with the derivatised surface of a microscope slide glass support. Various reactive functionalities have been used to derivatise array surfaces, including isocyanates, N-hydroxysuccinimide esters and epoxides. The last are a popular choice due to their relative stability, particularly in aqueous environments, which means that they remain active even after periods of storage. However, a more cost effective method of utilising unmodified oligonucleotides attached to a glass surface via hydrogen bonding or electrostatic attraction has also been investigated (Call et al., 2001, Biotechniques. 30(2): 368-372, 374 and 376).

Other coupling approaches include acrylamide derivatised oligonucleotides which can be copolymerised with acrylamide and bis-acrylamide monomers to produce hydrogel spots on microarray surfaces (Yershov et al., 1996, PNAS USA 93: 4913-4918; Vasiliskov et al., 1999, Biotechniques 27: 592-594, 596-598, 600). Immobilisation of oligonucleotides in gel spots can increase the amount of probe oligonucleotide available for hybridisation by between 2 and 3 orders of magnitude with consequent improvements in sensitivity. In addition, the oligonucleotides are held tethered in an aqueous environment so that the physics of their hybridisation is much more like the behaviour of untethered oligonucleotides in solution. This is advantageous from the point of view of oligonucleotide design in the present invention as most design algorithms use hybridisation models based on the parameters of solution phase hybridisation.

Self assembling arrays comprising oligonucleotides synthesized on beads that are allowed to passively “settle” into a pitted surface have also been reported (Oliphant et al., 2002, Biotechniques Suppl: 56-58, 60-51; Ferguson et al., 2000, Anal Chem 72(22): 5618-5624; Steemers et al., 2000, Nat Biotechnol. 18(1): 91-94).

Any of the above coupling or array construction methods are suitable for use in the present invention.

Arrays or microarrays of oligonucleotides according to the invention have a number of applications. In particular, the microarrays allow reliable analysis of different mRNA splice variants. Attempts have been made by various groups to produce microarrays that can resolve different alternative splice variants (see Kane et al., 2000, Nucleic Acids Res. 28: 4552-4557) but these prior art approaches have high levels of cross-hybridisation between different splice variants. Arrays comprising the oligonucleotides of this invention have reduced levels of cross-hybridisation compared with the prior art methods.

### Further Support-Bound Probes

While microarrays are a particularly useful application for the oligonucleotides of this invention, other suitable methods of employing oligonucleotide probes linked to a solid support are known in the art. For example, oligonucleotides probes may be coupled to magnetic beads. Avidin labelled magnetic microparticles are available from a number of sources, for example Avidin Dynabeads® (Invitrogen, Inc., San Diego, Calif., USA) or Sera-Mag® Streptavidin beads (Seradyn, Inc., Indianapolis, Ind., USA). Biotinylated oligonucleotide probes according to the invention can be readily coupled to avidinated beads. Labelled samples can be contacted with probe-coupled magnetic particles under hybridising conditions. The magnetic beads then permit facile separation of hybridised from unhybridised probes by using a magnet to retain beads in the reaction vessel while removing the solution phase, unbound probes. Non-specifically bound probes can then be washed and the wash buffer can be removed while magnetically retaining the probe/particle complexes. The wash process can be repeated multiple times. The captured labelled sample material can then be eluted for detection or it can detected in situ depending on the mode of detection used.

In a further embodiment, fluorescently encoded microparticles may be used, for example the xMAP system from Luminex, Inc (Austin, Tex., USA). The xMAP system provides an array of up to 100 independently resolvable fluorescent beads that can be detected in a flow sorting device. This technology enables multiple hybridisation probes to be labelled with distinct encoded fluorescent beads for multiplex nucleic acid detection applications (see Dunbar, 2006, Clin Chim Acta. 363(1-2): 71-82; Flagella et al., 2006, Anal Biochem. 352(1): 50-60). This technology is very well suited to multiplexed detection of multiple targets using the probes of this invention.

### Amplification and Labelling of Nucleic Acids

Techniques for labelling nucleic acids in biological samples are known in the art. For the purposes of the present invention, genomic DNA and mRNA are common targets and non-limiting techniques for labelling these molecules in particular are discussed below.

Genomic DNA may be amplified by cloning genomic DNA into bacterial plasmids, which can then be amplified in a bacterial vector. Whole genome amplification can also be achieved using Multiple Displacement Amplification (MDA), which is a non-specific amplification approach that allows large amounts of genomic DNA to be produced in a single reaction with generic primers (Hosono et al., 2003, Genome Res. 13(5): 954-64).

Specific amplification of genomic DNA can be achieved using PCR. In addition, PCR can be used to introduce labels into the amplified genomic DNA by linking labels to the primers used in the amplification reaction.

Amplification of mRNA may be achieved by a number of methods known in the art.

Suitable methods include in vitro transcription (IVT). There are various methods of IVT RNA amplification and labelling (see for example: Moll et al., 2004, Anal Biochem. 334(1): 164-174). In such methods, RNA is labelled by incorporation of tagged nucleotides during the IVT reaction.

In addition, PCR-based methods of mRNA amplification are known in the art (see for example Herrler et al. 2000, J Mol Med. 78(7): B23; Petalidis et al., 2003, Nucleic Acids Res. 31(22): e142) and may be used in the present invention.

### Further Applications of Labelled Oligonucleotides

In certain embodiments of the invention, labelled oligonucleotides may have the following applications. Leukemia is characterised by a number of chromosomal translocations, and specific translocations have prognostic implications. There are a number of methods for detecting these translocations but a currently important method is Fluorescence In Situ Hybridisation (FISH). In this technique, intact chromosomes are contacted with labelled probes that bind to specific sequences on the chromosomes allowing the translocations to be identified and the leukemia to be characterized (see for example: Dohner et al., 1997, Leukemia 11(S2): S19-24). The oligonucleotides of the invention would allow enhanced discrimination of the FISH method.

In another application, the oligonucleotides of the invention may be molecular beacons. Molecular beacons are fluorescent probes that fluoresce only in the presence of their target. Molecular beacons can be used in RT-PCR for detection of the amplification products (Tyagi and Kramer, 1996, Nat Biotechnol. 14(3): 303-308). In addition, molecular beacon microarrays can be constructed. These would have the advantage that the target nucleic acid would not have to be labelled and only oligonucleotide-bound material would be detected, in contrast to conventional microarrays where non-specifically adsorbed labelled target is also detected.

### Antisense Oligonucleotides

The oligonucleotides of the invention may be used as antisense compounds with enhanced specificity. Antisense compounds operate by various modes of action. One mode of action of particular interest here is the use of antisense oligonucleotides to modulate alternative splicing (Sazani & Kole, 2003, J Clin Invest. 112(4): 481-6). Antisense sequences complementary to exon/intron boundaries of unspliced mRNA may prevent binding of splicing factors thus inhibiting formation of the spliceasome. This can direct splicing to alternative splice sites. With two antisense oligonucleotides targeting both exon/intron boundaries for a single exon, an exon can be skipped in its entirety.

Exemplary genes that are good targets for splice modulation are transmembrane receptors (such as the TNF-alpha receptor gene) that can be produced in a soluble form by alternative splicing to remove a transmembrane domain. Other targets include genes that regulate apoptosis such as bcl-x. Bcl-xL and bcl-xS are generated by alternative splicing of bcl-x intron 2, via the use of a common 3′ splice site and two alternative 5′ splice sites. Bcl-xL, the longer splice variant, is anti-apoptotic and high levels of bcl-xL have been correlated with resistance to chemotherapy (Liu et al., 1999, Am J Pathol. 155(6): 1861-7). The shorter variant, bcl-xS, which uses a 5′ splice site farther upstream from the 3′ splice site than bcl-xL, has been shown to have pro-apoptotic properties (Minn et al., 1996, J Biol Chem. 271(11): 6306-12). Oligonucleotides, targeted against the bcl-xL 5′ splice site were shown to shift splicing towards production of bcl-xS in various cancer cell lines (Taylor et al., 1999, Nat Biotechnol. 17(11): 1097-100; Mercatante et al., 2001, J Biol Chem. 276(19): 16411-7).

Oligonucleotides of the invention may be used to mediate RNase H based gene inhibition allowing selective suppression of specific splice variants of a given target gene. RNase H specifically degrades the RNA portion of DNA/RNA duplexes. RNase H appears to be a ubiquitous intracellular and extracellular nuclease that is postulated to provide an anti-viral defense mechanism for cells. The presence of this enzyme can be exploited to selectively degrade target ribonucleic acids in cells by designing single-stranded DNA oligonucleotides complementary to specific RNA sequences in cells. Antisense oligonucleotides of the invention can be designed to specifically to degrade particular splice variants of a gene with greatly reduced affinity for alternative splice variants that may comprise one the blocks of sequence in the target. This may be used to investigate the function of splice variants and as a therapeutic approach.

RNase H however only recognizes certain types of modified nucleic acids, so only certain analogues can be used in antisense molecules intended for an RNase H dependent effect. Phophorothioate backbones do recruit RNase to phosphorothioate oligonucleotide/RNA duplexes. However, certain sugar modifications prevent recruitment of RNase H to oligonucleotide/RNA duplexes. 2-O-methyl and larger modifications inhibit RNase H recruitment even if the native phosphodiester or phosphorothioate backbones are present. 2′-deoxy-2′-fluoro-beta-D-arabinonucleic acid is a 2′ modification that does support RNase H activity and is discussed below. Since many modifications, that are otherwise desirable for the enhancement of binding affinity or exonuclease stability, inhibit RNase H activity, a different approach to oligonucleotide design is necessary.

One approach to obtain the benefits of RNase H activity and the benefits of enhanced binding affinity and exonuclease stability, is to employ chimeric oligonucleotides. Chimeric oligonucleotides provide a window of sequence that supports RNase H activity flanked at the 3′ and 5′ ends by other nucleotides that introduce other desirable properties into the sequence (Monia et al., 1993, J Biol Chem. 268(19): 14514-22; Lok et al., 2002, Biochemistry 41(10): 3457-67). The RNase H recruiting region of the antisense oligonucleotide preferably comprise a length of between 5 and 7 nucleotides.

A suitable nucleic acid analogue for antisense applications is 2′-O-methoxyethoxy (2′-MOE), a 2′ sugar modification. Like 2′-O-methyl oligonucleotides, 2′-MOE oligonucleotides are also RNA analogues with enhanced affinity for RNA binding. They have good toxicity profiles in animal studies (Prakash et al., 2008, J Med Chem. 51(9): 2766-76). 2′-MOE oligonucleotides with a phosphorothioate backbone have shown promise as orally deliverable antisense agents (Tillman et al., 2008, J Pharm Sci. 97(1): 225-36).

A further suitable nucleic acid analogue for antisense applications is 2′-deoxy-2′-fluoro-beta-D-arabinonucleic acid (2′-F-ANA). This analogue has been shown to have enhanced RNA affinity, like other 2′-modified nucleotide analogues. In addition, 2′-F-ANA can also mediate RNase H cleavage of RNA in 2′-F-ANA/RNA duplexes. In addition, 2′-F-ANA show significant resistance to exonuclease degradation (Ferrari et al., 2006, Ann NY Acad Sci. 1082: 91-102). 2′-F-ANA analogues can also mediate siRNA (Dowler et al., 2006, Nucleic Acids Res. 34(6): 1669-75).

In a further embodiment, it has been shown that dsRNA-dependent protein kinase PKR, which is normally activated in virally infected cells to induce cell death, can be activated by antisense RNA. It has been shown that antisense RNA oligonucleotides of sufficient length to activate PKR complementary to chimeric mRNA fragments expressed by chimeric genes resulting from chromosomal translocations can be used to kill cancer cells that express mutated genes (Shir & Levitzki, 2002, Nat Biotechnol. 20(9): 895-900). Antisense oligonucleotides with the mismatch modifications of this invention designed to flank deletions or translocations in mutant genes to produce a dsRNA molecule are likely to be more specific than unmodified oligonucleotides for the purposes of inducing cell death in cancerous cells. This PKR-mediated killing strategy may be useful in treating many cancers that express a unique RNA species.

### Computer Simulation of Hybridisation of Various Oligonucleotide Probes

Oligonucleotide designs can be using evaluated using various software modelling tools. Examples of predicted melting curves generated using the open source software tool DINAMeIt (Markham & Zuker, supra; DINAMeIt version used: UNAFoId 3.6) are shown in FIGS. 10 to 15 and are discussed below.

DINAMeIt is a software tool that models the interaction between two nucleic acids. Unlike simple melting temperature models, DINAMeIt can simulate the entire annealing process between two complementary oligonucleotides over the entire temperature range of aqueous solutions, i.e. from 0 to 100° C. In addition, DINAMeIt can model the interaction of pairs of sequences that are not perfectly complementary, i.e. an oligonucleotide comprising a mismatch compared with its target sequence. DINAMeIt also accounts for self-interactions of individual sequences both in terms of intra-molecular interactions and inter-molecular interactions. This allows evaluation of oligonucleotide probes such as molecular beacons that are designed to form stable structures in the absence of target.

Thus DINAMeIt is well suited to assess oligonucleotide probe designs according to the invention, which is a useful exercise prior to synthesis of such oligonucleotides. In following examples, DINAMelt was used to show the effect of various oligonucleotide designs using the BCR-ABL gene fusion as a model system. A characteristic B2A2 fusion between the BCR and ABL genes found in numerous leukemias (Bohlander 2000, supra) was selected as a model target to asses distinguish from the parent BCR and ABL genes.

Sequences of the B2A2 mRNA domains extending 25 bases flanking the B2A2 fusion junction (i.e. domain junction), corresponding parent BCR and ABL sequences, and exemplar oligonucleotide probe designs are shown in Table 1.

TABLE 1
B2A2, ABL, BCR and exemplar oligonucleotides
used for DINAMelt analysis

| Name | Sequence (shown 5′-3′) |
|---|---|
| B2A2 | ATTCCGCTGACCATCAATAAGGAAGAAGCCCTTCAGCGGCCAGTAGCATC |
| | [SEQ ID NO: 9] |
| ABL | CACTGCAATGTTTTTGTGGAACATGAAGCCCTTCAGCGGCCAGTAGCATC |
| | [SEQ ID NO: 10] |
| BCR | ATTCCGCTGACCATCAATAAGGAAGATGATGAGTCTCCGGGGCTCTATGG |
| | [SEQ ID NO: 11] |
| PROBE1 | TACTGGCCGCTGAAGGGCTTCTTCCTTATTGATGGTCAGC |
| | [SEQ ID NO: 12] |
| PROBE2 | TACTGGCCGCCGAAGGGCTTCTTCCTTATCGATGGTCAGC |
| | [SEQ ID NO: 13] |
| PROBE3 | GGCCGCTGAAGGGCTTCTTCCTTATTGATGGTCAGCGGAA |
| | [SEQ ID NO: 14] |
| PROBE4 | GGCCGCCGAAGGGCTTCTTCCTTATTGATTGTCAGCGGAA |
| | [SEQ ID NO: 15] |
| PROBE5 | GCCGCTGAAGGGCTTCTTCCTTATTGATGG |
| | [SEQ ID NO: 16] |
| PROBE6 | GCCGCTGTAGGGCTTCTTCCTTTTTGATGG |
| | [SEQ ID NO: 17] |
| PROBE7 | CCGCTGAAGGGCTTCTTCCTTATTGATGGTCAGC |
| | [SEQ ID NO: 18] |
| PROBE8 | CCGCTGTAGGGCTTCTTCCTTATTGTTGGTCAGC |
| | [SEQ ID NO: 19] |

In the B2A2, ABL and BCR sequences shown above, the domain sequences shared by B2A2 and ABL are shown underlined, while the domain sequences shared by B2A2 and BCR are shown in bold. The design of oligonucleotides (PROBE1-PROBE8) to allow maximisation of discrimination between the sequences is discussed below.

In these examples, DINAMeIt has been used to calculate the melting curves for the interaction of the oligonucleotides (PROBE1 to PROBE8) listed in Table 1 with the three targets (B2A2, BCR and ABL). The oligonucleotides are all designed to bind to B2A2 but can cross-hybridise with ABL and BCR because of the presence of shared domain sequence. In the PROBE sequences, parts of the oligonucleotides that can bind to ABL are shown underlined while the portions of the oligonucleotides that can bind to BCR are shown in bold. Discontinuities in the oligonucleotide sequences are shown in italics.

DINAMeIt calculates melting curves for the interaction of two sequences only using a relatively small number of user-defined parameters: the sequence of each oligonucleotide in the pair; oligonucleotide concentration; type of oligonucleotide (DNA or RNA); and salt concentration. Duplex simulations were carried out using equal concentrations of oligonucleotide and target. The concentration of oligonucleotides or target was set to be 10⁻⁵ molar. DINAMeIt can simulate DNA duplex hybridisations with different concentrations of sodium and magnesium ions. In the DINAMeIt version we used, for RNA hybridisations the parameters only support simulations in 1M sodium with no correction available for magnesium. In Examples 1 to 4 below, salt (NaCl) concentration was set to be 50 mM and oligonucleotide probes and target were assumed to be DNA. In Examples 5 and 6, salt (NaCl) was set to 1M to evaluate the effect of salt on DNA hybridisation and to evaluate the hybridisation of RNA, respectively. In each of Examples 1 to 6, the concentration of MgCl₂ was set to zero.

### EXAMPLE 1

### Symmetric Length Probes

In the first modelling example, a relatively simple oligonucleotide design was employed to allow discrimination of the B2A2 target from the parent cross-hybridising sequences. A 40-mer probe was designed so that 20 bases of the oligonucleotide match the sequence from BCL and 20 bases of the oligonucleotide match the sequence from ABL. This is referred to as a “symmetric length” or “symmetric length probe” design, in that portions of the oligonucleotide probe are the same length on either side of the portion junction, and is used in the prior art to design so-called “junction probes” for microarrays (Su et al., 2008, BMC Genomics. 9: 273; Johnson et al., 2003, Science. 302(5653): 2141-2144; Kane et al., 2000, Nucleic Acids Res. 28: 4552-4557).

PROBE 1 as shown in Table 1 was designed to be perfectly complementary to the target fusion sequence B2A2. This also means that 20 bases of PROBE 1 are complementary to the BCR gene and 20 bases are complementary to the ABL gene. PROBE2 as shown in Table 1 is also a 40-mer designed to bind to the B2A2 sequence at the junction between the BCR and ABL donor-sequences. PROBE2, however, has two mismatches introduced into the sequence at the 10^{th} position downstream from the junction between the BCR and ABL sequences in the B2A2 sequence. Thus, only 19 bases of PROBE2 are complementary to the BCR sequence and only 19 bases of PROBE2 are complementary to the ABL sequence. In Table 1, the PROBE2 mismatches are marked in italics. By comparison with PROBE1, it can be seen that two T residues have been changed to C residues in the mismatched PROBE2.

The melting curve predicted by DINAMeIt for the hybridisation of PROBE1 to its intended target B2A2 is shown in FIG. 10 as a dense solid line and the melting curves for the hybridisation of PROBE1 to ABL and BCR under the same hybridisation conditions are shown in FIG. 10 as dense dashed lines. The graph shows the mole fraction that is present as a duplex on the y-axis, i.e. the fraction of the starting quantities of oligonucleotide and target that are duplexed for a given temperature on the x-axis. The curves for the hybridisation of each oligonucleotide probe with the three targets are overlaid On the same graph to give an idea of the expected amount of cross-hybridisation for a given set of hybridisation conditions if the B2A2 target and cross-hybridising BCR and ABL sequences were hybridised under the same conditions. While this is a simulated experiment, it allows for an assessment of relative specificity of different oligonucleotide probe designs.

The melting curve for the hybridisation of PROBE2 to its intended target B2A2 is shown as a light solid line in FIG. 10 and the melting curves for hybridisation of PROBE2 to ABL and BCR are shown in FIG. 10 as light dashed lines to enable comparison with the non-mismatched probe design.

Various features of the invention are illustrated by the melting curves shown in FIG. 10. It can be seen by comparison of the melting curves of PROBE1 and PROBE2 that the introduction of the mismatches into PROBE2 reduces the overall binding affinity of PROBE2 compared with PROBEI as the PROBE2 curves are shifted to the left, i.e. to a lower temperature profile. Similarly, it can be seen that there is significant binding of PROBE1 to both the BCR and ABL sequences. In particular, there is very significant binding of PROBE1 to the ABL sequence. Although the overall binding affinity of PROBE2 is reduced compared to PROBE1, the difference in binding affinity between PROBE2 bound to its intended B2A2 target and PROBE2 bound to either the ABL or BCR sequence is increased compared to PROBE1.

The results in FIG. 10 can be quantitatively compared in various ways. For example, hybridising PROBE1 at about 74.5° C. with its intended target in the presence of the ABL and BCR targets yields a mole fraction of PROBE1 that binds to ABL of about 0.5×10⁻⁶ while the mole fraction of PROBE1 binding to its intended target is about 2.9×10⁻⁶. The ratio of PROBE1 binding with B2A2 to PROBE1 binding with ABL is thus 5.8:1. The mole fraction binding to BCR would be negligible at this temperature.

In contrast, when PROBE2 is hybridised at 68° C. in the presence of the B2A2 target and the cross-hybridising targets, the mole fraction of PROBE2 that binds to ABL is about 0.5×10⁻⁶ while the mole fraction of PROBE2 binding to its intended target is about 4.8×10⁻⁶. The ratio of PROBE2 binding with B2A2 to PROBE2 binding with ABL is thus 9.6:1. Again, the mole fraction binding to BCR would be negligible. This difference between PROBE1 and PROBE2 clearly illustrates the advantage of introducing mismatches into oligonucleotide probes to enhance discrimination.

### EXAMPLE 2

### Symmetric Tₘ Probes

In a second example, an alternative oligonucleotide probe design is employed to enable higher levels of discrimination of the B2A2 target from the parent targets, ABL and BCR. Here, a 40-mer oligonucleotide probe was designed so that the Tₘ (calculated using the Nearest Neighbour method) of the oligonucleotide portion that hybridises to the sequence from BCL is as similar as possible to the portion of the oligonucleotide that hybridises to the sequence from ABL in the probe. We refer to this as a symmetric Tm design. While the Tₘs of the oligonucleotide probe portions are similar, the lengths of the portions will typically differ in order to make the portion Tₘs symmetrical, hence the probes are typically or often asymmetric with regard to the relative lengths of the two oligonucleotide portions.

PROBE3 shown in Table 1 was designed to be perfectly complementary to its target. In addition, a 24 base portion of PROBE3 is complementary to the BCR gene while a 16 base portion of PROBE3 is complementary the ABL gene. The Tₘs of these probe portions were designed to be as similar as possible. PROBE4 shown in Table 1 is also a 40-mer oligonucleotide designed to bind to the B2A2 sequence at the junction between the BCR and ABL sequences. PROBE4, however, has two mismatches introduced into the sequence at position 7 from the 5′-end of the oligonucleotide and at position 30 from the 5′-end of the oligonucleotide. These mismatches are shown in italics. Therefore, 22 bases of PROBE4 binds to the BCR sequence and 11 bases of PROBE4 binds to the ABL sequence. By comparison with PROBE3, it can be seen that a T residue and a G residue have been changed to a C and a T residue, respectively, in the mismatched PROBE4 sequence. These mismatches have been introduced so that the melting temperatures of the sub-sequences on either side of the mismatch within each probe portion are as close to each other as possible. This has the effect of minimising the melting temperatures of subsequences (or subportions) on either side of the mismatch.

In FIG. 11, the melting curve for the hybridisation of PROBE3 to its intended target B2A2 is shown as a dense solid line and the melting curves for hybridisation of PROBE3 to ABL and BCR are shown as dense dashed lines. The melting curve for the hybridisation of PROBE4 to its intended target B2A2 is shown as a light solid line and the melting curves for hybridisation of PROBE4 to ABL and BCR are shown as light dashed lines.

Various features of the improved oligonucleotide probe design are illustrated by the melting curves shown in FIG. 11. As with the symmetric length design in Example 1, it can be seen by comparison of the melting curves of PROBE3 and PROBE4 that the introduction of the mismatches into PROBE4 reduces the overall binding affinity of PROBE4 compared with PROBE3 as the PROBE4 curve is shifted to the left, i.e. to a lower temperature profile. Similarly, by comparison with Example 1 it can be seen that there is still significant binding of PROBE3 to both the BCR and ABL sequences. However, there is considerably less binding of PROBE3 to the ABL sequence compared with the binding of PROBE1 with the ABL sequence. In contrast, binding of PROBE3 to the BCR sequence has increased so that it shows a pattern of binding that is very similar to that of PROBE3 binding to the ABL sequence. In short, the symmetric Tₘ probe design has reduced the differences in the amount of cross-hybridisation between PROBE3 and the BCR and ABL targets when compared with PROBE1 but increased the average discrimination against both “off-target” hybridisations.

In FIG. 10, it could be seen that for a given mole fraction of non-specific binding, i.e. for a mole fraction of binding to either BCR or ABL of 0.5×10⁻⁶, the specific binding of the probes to B2A2 was relatively low, 2.9×10⁻⁶ and 4.9×10⁻⁶ for PROBE1 and PROBE2 respectively. In FIG. 11 it can be seen that the overall increase in discrimination enabled by the improved symmetric Tₘ probe design means that the mole fraction of specific probe binding to B2A2 for a given mole fraction of non-specific binding under the same conditions, e.g. 0.5×10⁻⁶, is now greater than 9.0×10⁻⁶ over a range of hybridisation temperatures, shown as the shaded area under the PROBE3 curve. However, for PROBE3 it can be seen that the range where specific binding is greater than 9.0×10⁻⁶ and non-specific binding is less than 0.5×10⁻⁶ is from 69.5° C. to 70.5° C., or about 1° C.

It can be seen from the melting curves of PROBE4 that although the overall binding affinity is reduced compared to PROBE3, the difference in binding affinity between the oligonucleotide bound to its intended B2A2 target and the oligonucleotide bound to either the ABL or BCR sequence is increased compared to PROBE3 (as is found in Example 1 for PROBE2 compared with PROBE1). As for PROBE3, this improvement in specificity of PROBE4 means that the mole fraction of specific oligonucleotide probe binding to B2A2 for a given mole fraction of non-specific binding, e.g. 0.5×10⁻⁶, is now greater than 9.0×10⁻⁶ over a range of hybridisation temperatures shown as the shaded area under the PROBE4 curve. In contrast to PROBE3, it can be seen for PROBE4 that the range where specific binding is greater than 9.0×10⁻⁶ and non-specific binding is less than 0.5×10⁻⁶ is from 59.5° C. to 63.5° C., or about 4° C.

Thus both PROBE3 and PROBE4 show a significant improvement in specificity for target sequence compared with PROBE1, which clearly demonstrates the advantage of the symmetric Tₘ design. In addition, the greater range of temperature at which high specificity can be achieved with PROBE4 compared with PROBE3 illustrates that the improved design also increases the “robustness” of the oligonucleotide probe. This difference between PROBE3 and PROBE4 clearly illustrates that there is still a further advantage gained by introducing mismatches into symmetric Tₘ-designed oligonucleotides to enhance discrimination.

### EXAMPLE 3

### Effect of Reducing Probe Length

In this example, a symmetric length design is employed again but using a shorter oligonucleotide probe compared to Example 1 to determine if shorter oligonucleotides show higher levels of discrimination of the B2A2 target from the parent targets, ABL and BCR.

PROBES as shown in Table 1 was designed to be perfectly complementary to its target. Thus, 15 bases of PROBE5 are complementary to the BCR gene while 15 bases of PROBE5 are complementary the ABL gene. PROBE6 shown in Table 1 has two mismatches introduced into the sequence at position 8 and at position 23 from the 5′-end of the oligonucleotide probe. The mismatches are shown in italics in Table 1. By comparison with PROBE5, it can be seen that two A residues have been changed to two T residues in the mismatched probe. PROBE6 thus has 14 bases complementary to the BCR sequence and 15 bases complementary to the ABL sequence.

In FIG. 12, the melting curve for the hybridisation of PROBE5 to its intended target B2A2 is shown as a dense solid line and the melting curves for the hybridisation of PROBE5 to ABL and BCR are shown as dense dashed lines. The melting curve for the hybridisation of PROBE6 to its intended target B2A2 is shown as a light solid line and the melting curves for hybridisation of PROBE4 to ABL and BCR are shown as light dashed lines.

Various features of the shortened symmetric length probe design are illustrated by the melting curves shown in FIG. 12. As with the longer symmetric length design in Example 1, it can be seen by comparison of the melting curves of PROBE5 and PROBE6 that the introduction of the mismatches into PROBE6 reduces the overall binding affinity of PROBE6 compared with PROBE5 as the PROBE6 curve is shifted to the left, i.e. to a lower temperature profile. Similarly, it can be seen that there is still significant binding of PROBE5 to both the BCR and ABL sequences. In particular, there is significant binding of PROBE5 to the ABL sequence although relatively less compared with the binding of PROBE1 with the ABL sequence. As with PROBE1, binding of PROBE5 to the BCR sequence is considerably less than the binding of PROBE5 to the ABL sequence. Thus, the reduced length of PROBE5 has enhanced discrimination against the cross-hybridising targets, BCR and ABL, when compared with PROBE1.

Similarly, it can be seen from the melting curves of PROBE6 that although the overall binding affinity is reduced compared to PROBE5, the difference in binding affinity between PROBE6 bound to its intended B2A2 target and PROBE6 bound to either the ABL or BCR sequence is increased compared to PROBE5 (as found in Example 1 for PROBE2 compared with PROBE1, respectively). Again, this improvement in specificity can be quantitatively compared as in Example 1. Thus, hybridising PROBE5 at about 69° C. with its intended target in the presence of the ABL and BCR targets yields a mole fraction of PROBE5 that binds to ABL is about 0.5×10⁻⁶ while the mole fraction of PROBES binding to its intended target is about 4.2×10⁻⁶. The ratio of PROBE5 binding with B2A2 to PROBE5 binding with ABL is thus 8.4:1. This is a significant improvement compared with PROBE1 and clearly demonstrates the advantage of a shortened probe. The mole fraction binding to BCR would still be negligible at this Tₘ though.

Similarly, if PROBE6 is hybridised at 62.5° C. in the presence of the B2A2 target and the cross-hybridising targets, the mole fraction of PROBE2 that binds to ABL is also about 0.5×10⁻⁶ while the mole fraction of PROBE2 binding to its intended target is almost 6×10⁻⁶. The ratio of PROBE2 binding with B2A2 to PROBE2 binding with ABL is thus 12:1. Again, the mole fraction binding to BCR would be negligible. This difference between PROBES and PROBE6 clearly illustrates that there is still a further advantage of introducing mismatches into oligonucleotide probes to enhance discrimination even in shorter oligonucleotide probes.

### EXAMPLE 4

### Shortened Symmetric Tₘ Probes

In this example, the symmetric Tₘ design was employed to enable higher levels of discrimination of the B2A2 target from the parent targets, ABL and BCR. A 34-mer oligonucleotide probe was designed so that the Tₘ of the oligonucleotide portion that hybridises to the sequence from BCL is as similar as possible to the portion that hybridises to the sequence from ABL. As before, Tm is calculated using the Nearest Neighbour method. The oligonucleotides are shown in Table 1 as PROBE7 and PROBE8.

PROBE7 was designed to be perfectly complementary to its target. In addition, 20 bases of PROBE7 are complementary to the BCR gene while 14 bases of PROBE7 are complementary the ABL gene. The Tₘs of these two oligonucleotide probe portions are designed to be as similar as possible. PROBE8 in Table 1 is also a 34-mer designed to bind to the B2A2 sequence at the boundary between the BCR and ABL sequences but has two mismatches introduced into the sequence at position 7 and at position 26 from the 5′-end of the oligonucleotide probe. In Table 1, these mismatches are shown in italics. By comparison with PROBE7, it can be seen that two A residues have been changed to T residues in PROBE8. The mismatches have been introduced so that the melting temperatures of the subsequences on either side of the mismatch within each oligonucleotide probe portion are as close to each other as possible. This has the effect of minimizing the Tₘs of the subsequences on either side of the mismatch.

In FIG. 13, the melting curve for the hybridisation of PROBE7 to its intended target B2A2 is shown as a dense solid line and melting curves for the hybridisation of PROBE7 to ABL and BCR are shown as dense dashed lines. The melting curve for the hybridisation of PROBE8 to its intended target B2A2 is shown as a light solid line and the melting curve for hybridisation of PROBE8 to ABL and BCR are shown as light dashed lines.

Various features of the shortened symmetric Tₘ design are illustrated by the melting curves shown in FIG. 13. As with the symmetric length design, it can be seen by comparison of the melting curves of PROBE7 and PROBE8 that the introduction of the mismatches into PROBE4 reduces the overall binding affinity of PROBE8 compared with PROBE7 as the PROBE8 curve is shifted to the left, i.e. to a lower temperature profile. Similarly, it can be seen that there is still significant binding of PROBE7 to both the BCR and ABL sequences. However, there is considerably less binding of PROBE7 to the ABL sequence compared with the binding of PROBE5 with the ABL sequence. In contrast, binding of PROBE7 to the BCR sequence has increased and shows a very similar profile to the binding of PROBE7 with the ABL sequence. In short, the symmetric Tₘ design has reduced the differences in the amount of cross-hybridisation between PROBE7 and the BCR and ABL targets when compared with PROBE5. It can be seen from the melting curves of PROBE8 that although the overall binding affinity is reduced compared to PROBE7, the difference in binding affinity between PROBE8 bound to its intended B2A2 target and the probe bound to either the ABL or BCR sequence is increased compared to PROBE7, as is found in the previous examples. Again, this improvement in specificity can be quantitatively compared as in Example 2.

In FIG. 12 for Example 3, it could be seen that for a given mole fraction of non-specific binding, i.e. for a mole fraction of binding to either BCR or ABL of 0.5×10⁻⁶, the specific binding of the oligonucleotide probes to B2A2 was relatively low, 4.2×10⁻⁶ and 6×10⁻⁶ for PROBE5 and PROBE6, respectively. However, in FIG. 13 it can be seen that the overall increase in discrimination enabled by the improved symmetric Tₘ probe design means that the mole fraction of specific probe binding of PROBE7 to B2A2 for a given mole fraction of non-specific binding under the same conditions, e.g. 0.5×10⁻⁶, is now greater than 9.0×10⁻⁶ over a range of hybridisation temperatures, shown as the shaded area under the PROBE7 curve. However, for PROBE7 it can be seen that the range where specific binding is greater than 9.0×10⁻⁶ and non-specific binding is less than 0.5×10⁻⁶ is from just over 64° C. to just over 65° C., or about 1° C.

As in previous examples when comparing mismatched oligonucleotide probes with perfect match oligonucleotide probes, it can be seen from the melting curves of PROBE8 in FIG. 12 that although the overall binding affinity is reduced compared to PROBE7, the difference in binding affinity between the probe bound to its intended B2A2 target and the probe bound to either the ABL or BCR sequence is increased compared to PROBE7. As for PROBE7, this improvement in specificity of PROBE8 means that the mole fraction of specific probe binding to B2A2 for a given mole fraction of non-specific binding, e.g. 0.5×10⁻⁶, is now greater than 9.0×10⁻⁶ over a range of hybridisation temperatures shown as the shaded area under the PROBE4 curve. In contrast to PROBE3, it can be seen for PROBE4 that the range where specific binding is greater than 9.0×10⁻⁶ and non-specific binding is less than 0.5×10⁻⁶ is from 56.5° C. to just over 59° C., or a little over 2.5° C. Thus both PROBE7 and PROBE8 show a significant improvement in specificity compared with PROBE5, which clearly demonstrates the advantage of the symmetric Tₘ design. In addition, the greater range of temperature at which high specificity can be achieved with PROBE8 compared with PROBE7 illustrates that the improved design also increases the “robustness” of the probe. Interestingly, the shorter symmetric Tₘ design doesn't show an improvement over the longer symmetric Tₘ 40-mer in Example 2 by the measures used here.

### EXAMPLE 5

### Effect of Increased Salt Concentration on Symmetric Tₘ 34-mer Probes

In this example, the effect of increasing salt in hybridisation reactions was evaluated using the 34-mer symmetric Tₘ designed PROBE7 and PROBE8. Thus hybridisation of PROBE7 and PROBE8 to B2A2, ABL and BCR was evaluated using 1M salt rather than 50 mM salt as used in Example 4.

The melting curves for PROBE7 and PROBE8 with their targets are shown in FIG. 14. By comparison with the results of Example 4 shown in FIG. 13, it can be seen in FIG. 14 that increasing the salt concentration generally increases the temperature profiles of all the interaction but a more careful analysis reveals that the effect is not homogenous. Using the same specificity criteria as in Example 4, i.e. determining the temperature range in which the mole fraction of oligonucleotide probe binding to the B2A2 target is greater than 9×10⁻⁶ and off-target binding of oligonucleotide probe to ABL and BCR is less than 0.5×10⁻⁶, these temperature ranges are increased at higher salt concentration. This means that specific binding can occur over a wider range: the “specific range” of both PROBE7 and PROBE8 have increased by approximately 1.5° C. with increased salt concentration but the high specificity range of PROBE8 is still greater than for PROBE7.

### EXAMPLE 6

### Effect of RNA Hybridisation on Symmetric Tₘ 34-mer Probes

In this example, the effect of switching to RNA hybridisation was evaluated using the 34-mer symmetric Tₘ design from Examples 4 and 5, i.e. the binding of PROBE7 and PROBE8 were evaluated as if they and their targets were RNA. As in Example 5, the hybridisation of PROBE7 and PROBE8 to B2A2, ABL and BCR was evaluated using 1M salt rather than 50 mM salt that was used in Example 4.

The melting curves for PROBE7 and PROBE8 with their targets under these conditions are shown in FIG. 15. By comparison with the results of Example 5 shown in FIG. 14, it can be seen in FIG. 15 that changing from DNA hybridisation to RNA hybridisation generally increases the temperature profiles of all the interactions further but again, a more careful analysis reveals that the effect is not homogenous. Using the same specificity criteria as in Example 5, i.e. determining the temperature range in which the mole fraction of oligonucleotide probe binding to the B2A2 target is greater than 9×10⁻⁶ and off-target binding of oligonucleotide probe to ABL and BCR is less than 0.5×10⁻⁶, it can be seen that these temperature ranges are further increased by switching from DNA to RNA, i.e. specific binding can occur over an even wider range. The increase in range is just over 1° C. for both PROBE7 and PROBE8. PROBE7 now binds with the desired degree of specificity and sensitivity over a range of just over 4° C. (the lighter shaded region in FIG. 15, which overlaps with PROBE7). PROBE8 now binds with the desired degree of specificity with a range of 5° C. (the darker shaded region in FIG. 15), so the high specificity range of PROBE8 is still greater than for PROBE7.

As discussed above, one aspect of the invention is to employ nucleic acid analogues with enhanced binding affinity compared to natural phosphodiester deoxyribosenucleic acids, particularly RNA analogues such as 2′-O-methyl RNA or LNA. It is expected that these analogues will have a similar effect on probe binding as the switch to RNA shown in Example 6, i.e. there will be a general increase in the Tₘ of the oligonucleotide probe compared to the corresponding DNA oligonucleotide probe along with an increase in specificity when used against RNA targets.

### FRET Studies of Probe Hybridization

It is possible to experimentally evaluate probe designs according to this invention using a thermal cycler with a fluorescence detection capability. In the examples below, the hybridization of synthetic targets labeled with the dye Rhodamine (ROX) to synthetic probes labeled with the dye Fluorescein (FAM) was followed by measuring Fluorescence Resonance Energy Transfer (FRET) on a BioRad MiniOpticon Real Time PCR machine (BioRad Inc, Hercules, Calif., USA) as the temperature of the reaction was increased. During FRET, hybridization of the FAM probe to the ROX target results in loss of FAM signal and increase in ROX signal when the probe is excited at the FAM excitation frequency.

The following examples, like the DINAmelt examples above, evaluate the effect of various probe designs using the BCR-ABL gene fusion as a model system. The sequences of the synthetic oligonucleotides used in the experiments below are shown in Table 2.

TABLE 2
Synthetic oligonucleotides used for FRET analysis

| Name | Sequence (shown 5′-3′) |
|---|---|
| B2A2_FRET | ATTCCGCTGACCATCAAT^{ROX}AAGGAAGAAGCCCTTCAGCGGCCAGTAGCATC |
| | [SEQ ID NO: 20] |
| ABL_FRET | CACTGCAATGTTTTTGTGGAACAT^{ROX}GAAGCCCTTCAGCGGCCAGTAGCATC |
| | [SEQ ID NO: 21] |
| BCR_FRET | ATTCCGCTGACCATCAAT^{ROX}AAGGAAGATGATGAGTCTCCGGGGCTCTATGG |
| | [SEQ ID NO: 22] |
| PROBE9 | TACTGGCCGCTGAAGGGCTTCT^{FAM}TCCTTATTGATGGTCAGC |
| | [SEQ ID NO: 23] |
| PRQBE10 | TACTGGCCGCCGAAGGGCTTCT^{FAM}TCCTTATCGATGGTCAGC |
| | [SEQ ID NO: 24] |
| PROBE11 | GGCCGCTGAAGGGCTTCT^{FAM}TCCTTATTGATGGTCAGCGGAA |
| | [SEQ ID NO: 25] |
| PROBE12 | GGCCGCCGAAGGGCTTCT^{FAM}TCCTTATTGATTGTCAGCGGAA |
| | [SEQ ID NO: 26] |
| PROBE13 | GCCGCTGAAGGGCTTCT^{FAM}TCCTTATTGATGG |
| | [SEQ ID NO: 27] |
| PROBE14 | GCCGCTGTAGGGCTTCT^{FAM}TCCTTTTTGATGG |
| | [SEQ ID NO: 28] |
| PROBE15 | CCGCTGAAGGGCTTCT^{FAM}TCCTTATTGATGGTCAGC |
| | [SEQ ID NO: 29] |
| PROBE16 | CCGCTGTAGGGCTTCT^{FAM}TCCTTATTGTTGGTCAGC |
| | [SEQ ID NO: 30] |
| PROBE17 | gCcgCtgAagGgcTtcTt^{FAM}cCttAttGatGg |
| | [SEQ ID NO: 31] |
| PROBE18 | gCcgCtgTagGgcTtcTt^{FAM}cCttTttGatGg |
| | [SEQ ID NO: 32] |
| PROBE19 | CcgCtgAagGgcTtcTt^{FAM}cCttAttGatGgtCagC |
| | [SEQ ID NO: 33] |
| PROBE20 | CcgCtgTagGgcTtcTt^{FAM}cCttAttGttGgtCagC |
| | [SEQ ID NO: 34] |

The probes in Table 2 are designed to bind to B2A2 but can cross-hybridize with ABL and BCR because of the presence of shared portions of sequence. The portion of sequence shared by B2A2 and ABL is shown underlined, while the portion of sequence shared by B2A2 and BCR is shown in bold. The design of probes to maximize the discrimination of the B2A2 probe for its target over the two parent sequences is discussed below. The Tₘs of the portions of the sequence corresponding to each target are discussed below although only the perfect match probe sequences have been calculated. Probes 17 to 20 contain LNA nucleotides. In these sequences, LNA nucleotides are marked in upper case while normal DNA nucleotides are in lower case.

Probes and target oligonucleotides were made up in stock solutions of 50 pmol/μl. Hybridization reactions were made up as follows:

1 μl of Probe

1 μl of Target

5 μl of 10× Hybridization Buffer

43 μl of water to make reaction up to 50 μl.

Reactions were made up in BioRad white 0.1 ml reaction tubes. Optically clear caps were used to allow the reactions to be followed. Two different hybridization buffers were used:

10× Sodium only Hybridization Buffer (Na) comprising 500 mM NaCl; 200 mM TRIS HCl (pH 7.5) in water.

10× Magnesium-containing Hybridization Buffer (Mg) comprising 100 mM MgCl₂; 250 mM NaCl; 200 mM TRIS HCl (pH 7.5) in water.

The following protocol was used on the thermal cycler for all experiments:

1. Hold 3 minutes at 98° C.;

2. Drop to 20° C. hold for 30 seconds;

3. Record fluorescence in FAM channel

4. Increase by 0.5° C. and hold for 30 seconds

5. Record fluorescence in FAM channel

6. Repeat step 4 and 5 until 98° C. is reached

7. Drop to 4° C. and hold.

Note that although the dye Rhodamine was used for the FRET analysis, the FRET reaction was monitored with the FAM calibration supplied with the MiniOpticon instrument as the changes in the FAM channel gave the best signals.

For melting curve studies, the results are recorded as fluorescence difference curves (dF/dT curves), i.e. the difference between the fluorescence intensities of successive measurements at slightly different melting temperatures. Essentially, this is the derivative of the raw fluorescence intensity data.

For comparison with the earlier results, DINAmelt melt curves can also be analysed in a similar fashion. FIG. 16 shows a mole fraction difference curve (dM/dT curve) that corresponds to the melting curves for PROBE1 shown in FIG. 10. Again, the first derivatives of the melt curves in FIG. 10 are shown in FIG. 16.

FIG. 17 illustrates a typical set of dF/dT curves for the hybridization of PROBE9 to its intended target, B2A2_FRET (solid line), and the corresponding hybridizations to the ABL_FRET target (Dot-Dashed line) and BCR_FRET target (Dashed line), which share half their sequences with B2A2_FRET. The maximum points on the dF/dT curves can be regarded as the melting temperatures of the corresponding probe/target duplex. For the comparison of the specificity of different probe designs we define the Discriminating Difference in melt Temperature (DdT) as the difference between the melting temperature of the hybridization of the probe with the correct B2A2_FRET target and the melting temperature of the probe with the nearest cross-hybridizing target. In FIGS. 16 and 17, this is illustrated as the difference in the peaks of the dM/dT or dF/dT curves respectively for the B2A2 target and the nearest cross-hybridizing sequence, which for this particular set of targets is usually the ABL_FRET sequence.

Table 3 below lists the probes and the Tₘ determined from the dF/dT curves for the hybridization of each probe with the targets B2A2_FRET, ABL_FRET and BCR_FRET. The DdT values in Table 3 are defined as the difference between the melting temperature of the correct B2A2-FRET duplex and the next highest melting temperature for the cross-hybridizing duplexes of the probe with either ABL_FRET or BCR_FRET.

### EXAMPLE 7

### 40-mer DNA Symmetric Length Probes

As in Example 1, the symmetric length design approach is applied again, with the design of 40-mer probe such that 20 bases of the probe matches the sequence from BCL and 20 bases matches the sequence from ABL in the probe. By calculation with the program MELTING (Le Novère N, 2001, Bioinformatics. 17(12):1226-7) ₐₛsuming a concentration of 50 mM NaCl and a concentration of 1 microMolar of both oligonucleotides, the 5′ segment complementary to ABL has a Tₘ of 62.3° C., while the 3′ segment complementary to BCR has a Tₘ of 52.1° C. These clearly differ quite substantially.

The dF/dT curves measured for the hybridization of PROBE9 to its intended target B2A2_FRET is shown in FIG. 17 as the dense solid line and the dF/dT curves for the hybridization of PROBE1 to ABL and BCR under the same hybridization conditions are shown in FIG. 17 as the dense dashed lines. The graph shows the change in fluorescence intensity (dF/dT) on the y-axis. The curves for the hybridization of the probe with the three targets are overlaid on the same graph to give an idea of the amount of cross hybridization to expect for a given set of hybridization conditions if the B2A2 target and cross-hybridizing BCR and ABL sequences were hybridized under the same conditions.

TABLE 3
List of probes and Tₘ (in ° C.) determined from the dF/dT curves for the
hybridization of each probe with the targets B2A2_FRET, ABL_FRET and BCR_FRET

| | | | | Backbone | | | | | Discriminating |
|---|---|---|---|---|---|---|---|---|---|
| Probe | Design | Probe Structure | Length | Chemistry | Buffer | B2A2 Tₘ | ABL Tₘ | BCR Tₘ | deltaT (DdT) |
| PROBE9 | Symmetric length | Perfect Match | 40 | DNA | Na | 70 | 67 | 47 | 3 |
| PROBE9 | Symmetric length | Perfect Match | 40 | DNA | Mg | 76.5 | 75.5 | 60 | 1 |
| PROBE10 | Symmetric length | Artificial Mismatch | 40 | DNA | Na | 64.5 | 60 | 40.5 | 4.5 |
| PROBE10 | Symmetric length | Artificial Mismatch | 40 | DNA | Mg | 71.5 | 69 | 53 | 2.5 |
| PROBE11 | Symmetric Tₘ | Perfect Match | 40 | DNA | Na | 71 | 63 | 56.5 | 8 |
| PROBE11 | Symmetric Tₘ | Perfect Match | 40 | DNA | Mg | 77.5 | 72.5 | 67 | 5 |
| PROBE12 | Symmetric Tₘ | Artificial Mismatch | 40 | DNA | Na | 63 | 53.5 | 47 | 9.5 |
| PROBE12 | Symmetric Tₘ | Artificial Mismatch | 40 | DNA | Mg | 70 | 63 | 58 | 7 |
| PROBE13 | Symmetric length | Perfect Match | 30 | DNA | Na | 64.5 | 60 | 32.5 | 4.5 |
| PROBE13 | Symmetric length | Perfect Match | 30 | DNA | Mg | 72 | 70 | 47 | 2 |
| PROBE14 | Symmetric length | Artificial Mismatch | 30 | DNA | Na | 57 | 52 | 26 | 5.0 |
| PROBE14 | Symmetric length | Artificial Mismatch | 30 | DNA | Mg | 66.5 | 62 | 41 | 4.5 |
| PROBE15 | Symmetric Tₘ | Perfect Match | 34 | DNA | Na | 66.5 | 56.5 | 46.5 | 10 |
| PROBE15 | Symmetric Tₘ | Perfect Match | 34 | DNA | Mg | 73.5 | 66.5 | 60.5 | 7 |
| PROBE16 | Symmetric Tₘ | Artificial Mismatch | 34 | DNA | Na | 58 | 47.5 | 38 | 10.5 |
| PROBE16 | Symmetric Tₘ | Artificial Mismatch | 34 | DNA | Mg | 66.5 | 56.5 | 52 | 10 |
| PROBE17 | Symmetric length | Perfect Match | 30 | LNA | Na | 76 | 68 | 42 | 8 |
| PROBE17 | Symmetric length | Perfect Match | 30 | LNA | Mg | 86 | 81.5 | 63 | 4.5 |
| PROBE18 | Symmetric length | Artificial Mismatch | 30 | LNA | Na | 68 | 58.5 | 34.5 | 9.5 |
| PROBE18 | Symmetric length | Artificial Mismatch | 30 | LNA | Mg | 79 | 73.5 | 56.5 | 5.5 |
| PROBE19 | Symmetric Tₘ | Perfect Match | 34 | LNA | Na | 80.5 | 67 | 62 | 12.5 |
| PROBE19 | Symmetric Tₘ | Perfect Match | 34 | LNA | Mg | 88.5 | 78 | 74.5 | 9.5 |
| PROBE20 | Symmetric Tₘ | Artificial Mismatch | 34 | LNA | Na | 72.5 | 55.5 | 55 | 17 |
| PROBE20 | Symmetric Tₘ | Artificial Mismatch | 34 | LNA | Mg | 81.5 | 68 | 69.5 | 12 |

While this is an artificial scenario, it allows for an assessment of relative specificity of different probe designs.

PROBE9 is designed to be perfectly complementary to its target (B2A2_FRET). Similarly, 20-bases of PROBE9 will be complementary to either the BCR gene or the ABL gene. PROBE10 in Table 2 is also a 40-mer designed to bind to the B2A2 sequence at the boundary between the BCR and ABL sequences so that 20 bases of PROBE10 binds to the BCR sequence and 20 bases of PROBE10 binds to the ABL sequence. PROBE10, however, has two mismatches introduced into the sequence at the 10^{th} position from the boundary between the BCR and ABL sequences on either side of the boundary. In Table 2, the first mismatch is marked as the bold C nucleotide in the underlined portion of the probe that binds to the ABL sequence while the second mismatch is also a C nucleotide that is not shown in bold, in the bold portion of the probe sequence that bind to the BCR sequence in the B2A2 target. By comparison with PROBE9, it can be seen that two T residues have been changed to C residues in the mismatched probe.

The advantage of introducing artificial mismatches according to this invention is illustrated by the “Discriminating deltaT” (DdT) values listed in Table 3. PROBE9 has a Tₘ of 70° C. for hybridization with B2A2_FRET and a DdT value of 3° C. in the sodium only (Na) buffer. PROBE9 has a Tₘ of 76.5° C. for hybridization with B2A2_FRET and a DdT value of only 1° C. in the buffer containing magnesium ions (Mg). Similarly, PROBE10 has a Tₘ of 64.5° C. for hybridization with B2A2_FRET and a DdT value of 4.5° C. in the Na buffer. Furthermore, PROBE10 has a Tₘ of 71.5 for hybridization with B2A2_FRET and DdT value of 2.5° C. in the Mg buffer. Thus, from Table 3 it can be seen that the introduction of the mismatches into PROBE10 reduces the overall Tₘ of PROBE10 compared with PROBE9 in both the Na and Mg buffer. However, there is an increase in DdT of PROBE10 in comparison to PROBE9 of about 1.5° C. for both types of hybridization buffer. This difference between PROBE9 and PROBE10 clearly illustrates the advantage of introducing mismatches into probes to enhance probe discrimination.

### EXAMPLE 8

### Symmetric Tₘ Probes

As for Example 2, the improved probe design according to this invention is employed to demonstrate higher levels of discrimination of the B2A2 target from the parent targets, ABL and BCR. Again, the 40-mer probe is designed using the symmetric Tₘ design and PROBE11 and PROBE12 in Table 2 have the same sequences as PROBE3 and PROBE4 in Table 1 respectively. Note that, while the Tₘs of the probe portions are as similar as possible (5′ ABL-segment is 59.1° C. and the 3′ BCR-segment is 58.5° C. as determined by MELTING using the same conditions as Example 7), the lengths of the probe portions differ, hence the probes may be asymmetric with regard to the relative lengths of the two probe portions.

Certain advantages of the symmetric Tₘ probe design according to the invention are illustrated by the DdT values listed in Table 3. PROBE11 has a Tₘ of 71° C. for hybridization with B2A2_{——}FRET and a DdT value of 8° C. in the Na buffer. PROBE9 has a Tₘ of 77.5° C. for hybridization with B2A2_FRET and a DdT value of 5° C. in the Mg buffer. Similarly, PROBE12 has a Tₘ of 63° C. for hybridization with B2A2_FRET and a DdT value of 9.5° C. in the Na buffer. Furthermore, PROBE12 has a Tₘ of 70 for hybridization with B2A2_FRET and DdT value of 7° C. in the Mg buffer. Thus, it can be seen by comparison of the DdT value of PROBE9 with PROBE11 and the corresponding DdT values of PROBE10 with PROBE12 that there is a significant increase in DdT values for the symmetric Tₘ probe design according to the invention. Moreover, the introduction of the mismatches into PROBE12 reduces the overall Tm of PROBE12 compared with PROBE11 in both the Na and Mg buffers. However, there is an increase in DdT of PROBE12 in comparison to PROBE11 of about 1.5 and 2° C. for the Na and Mg buffers respectively.

Thus both PROBE11 and PROBEI2 show an improvement in specificity compared with PROBE9, which clearly demonstrates an advantage of the symmetric Tₘ design. In addition, the increase of DdT of PROBE12 compared with PROBE11 illustrates that there is still a further advantage gained by introducing mismatches into probes designed with a symmetric Tₘ to enhance probe discrimination and that the combined introduction of artificial mismatches and symmetric Tₘ probe designs have an additive effect on probe specificity

### EXAMPLE 9

### Effect of Reducing Probe Length

Example 3 used DINAmelt to explore the specificity of a shorter probe compared to Example 1 to see if shorter probes show higher levels of discrimination of the B2A2 target from the parent targets, ABL and BCR. In the present example, PROBE13 and PROBE14 have the same sequences as PROBE5 and PROBE6 from Example 3. By calculation with the program MELTING using the same conditions as Example 7, the 5′ segment complementary to ABL has a Tₘ of 56.2° C., while the 3′ segment complementary to BCR has a Tₘ of 40.0° C. These clearly differ quite substantially.

An advantage of introducing artificial mismatches according to the invention is further illustrated by the DdT values listed in Table 3. PROBE13 has a Tₘ of 64.5° C. for hybridization with B2A2_FRET and a DdT value of 4.5° C. in the Na buffer. PROBE13 has a Tₘ of 72 for hybridization with B2A2_FRET and a DdT value of only 2° C. in the Mg buffer. Similarly, PROBE14 has a Tₘ of 57° C. for hybridization with B2A2_FRET and a DdT value of 5.0° C. in the Na buffer. Furthermore, PROBE14 has a Tₘ of 66.5 for hybridization with B2A2_FRET and DdT value of 4.5° C. in the Mg buffer. Thus, from Table 3 it can be seen that the introduction of the mismatches into PROBE14 reduces the overall Tₘ of PROBE10 compared with PROBE9 in both the Na and Mg buffer. However, there is an increase in DdT of PROBE14 in comparison to PROBE13 of about 0.5 and 2.5° C. for the Na and Mg hybridization buffers respectively. This difference between PROBE13 and PROBE14 clearly illustrates the advantage of introducing mismatches into shorter probes to enhance probe discrimination. As expected, the shortened probes have a lower Tₘ than the longer probes. In addition, the shorter probes appear to be slightly more specific, i.e. the 30-mer probes have higher DdT values than the corresponding 40-mer probes.

### EXAMPLE 10

### Shortened Symmetric Tₘ Probes

In this example, PROBE15 and PROBE16 have the same sequences as PROBE7 and PROBE8 from Example 4, to test the improved symmetric Tₘ probe design according to the invention to see if it will deliver higher levels of discrimination of the B2A2 target from the parent targets, ABL and BCR. Note that, while the Tₘs of the probe portions are as similar as possible (5′ ABL-segment is 52.0° C. and the 3′ BCR-segment is 52.1° C. as determined by MELTING using the same conditions as Example 7), the lengths of the probe portions will typically differ, hence the probes are often asymmetric with regard to the relative lengths of the two probe portions.

Advantages of the symmetric Tₘ probe design according to this invention are illustrated by the DdT values listed in Table 3. PROBE15 has a Tₘ of 66.5° C. for hybridization with B2A2_FRET and a DdT value of 10° C. in the Na buffer. PROBE15 has a Tₘ of 73.5° C. for hybridization with B2A2_FRET and a DdT value of 7° C. in the Mg buffer. Similarly, PROBE16 has a Tₘ of 58° C. for hybridization with B2A2_FRET and a DdT value of 10.5° C. in the Na buffer. Furthermore, PROBE16 has a Tₘ of 66.5 for hybridization with B2A2_FRET and DdT value of 10° C. in the Mg buffer. Thus, it can be seen by comparison of the DdT value of PROBE15 with PROBE13 and the corresponding DdT values of PROBE16 with PROBE14 that there is a significant increase in DdT values for the symmetric Tₘ probe design according to this invention, although the lengths of these symmetric Tₘ probes is slightly greater (34 bases) than for the symmetric length probes (30 bases). Moreover, the introduction of the mismatches into PROBE16 reduces the overall Tₘ of PROBE16 compared with PROBE15 in both the Na and Mg buffer. However, there is an increase in DdT of PROBE16 in comparison to PROBE15 of about 0.5 and 3° C. for the Na and Mg buffers respectively, again showing the benefit of the mismatch for enhancing discrimination.

The improved symmetric Tₘ design of the 34-mer probes also shows a small increase in DdT over the longer symmetric Tₘ 40-mer probe design in Example 8 by the measures used here.

### EXAMPLE 11

### Effect of 2′-Modification on the Hybridization of Symmetric Length 30-mer Probes

As discussed herein, probes designed according to this invention can benefit from 2′ sugar modifications, which increase binding affinity but also increase sensitivity of the probes to structural deformation of probe/target duplexes. One preferred modification is Locked Nucleic Acid (Nielsen et al., 1999, J Biomol Struct Dyn. 17(2):175-91; Koshkin and Wengel, 1998, J. Org. Chem. 63: 2778-2781, 1998; Koshkin et al., 1998, J. Am. Chem. Soc. 120:13252-13253; Kvaerno and Wengel, 1999, Chem. Commun. 657-658; Jensen et al., 2001, J. Chem. Soc., Perkin Trans. 2: 1224-1232). PROBE17 and PROBE18 use the same symmetric length probe design as Examples 3 and 9, with a number of LNA nucleotides introduced into the sequence. LNA nucleotides and oligonucleotides are supplied by Exiqon A/S (Vedbaek, Denmark). Melting Temperatures for LNA/DNA oligonucleotide duplexes can be calculated using a tool on the Exiqon website (http://www.exiqon.com/oligo-tools). The Exiqon model does not take into account salt or oligonucleotide concentration and so the prediction is approximate. According to the Exiqon Tₘ model, the 5′ segment of PROBE17 complementary to ABL has a Tₘ of 76° C., while the 3′ segment complementary to BCR has a Tₘ of 60° C. These clearly differ quite substantially.

An advantage of combining artificial mismatches with LNA according to this invention is illustrated by the DdT values listed in Table 3. PROBE17 has a Tₘ of 76° C. for hybridization with B2A2_FRET and a DdT value of 8° C. in the Na buffer. PROBE17 has a Tₘ of 86 for hybridization with B2A2_FRET and a DdT value of only 4.5° C. in the Mg buffer. Similarly, PROBE18 has a Tₘ of 68° C. for hybridization with B2A2_FRET and a DdT value of 9.5° C. in the Na buffer. Furthermore, PROBE18 has a Tₘ of 79° C. for hybridization with B2A2_FRET and DdT value of 5.5° C. in the Mg buffer.

By comparison of PROBE13 with PROBE17 and PROBE14 with PROBE18, it can be seen that the introduction of LNA residues into PROBE17 and PROBE 18 greatly enhances the discrimination of PROBE17 and PROBE18 over the DNA-only probes. In addition, introduction of the mismatches into PROBE18 reduces the overall Tₘ of PROBE18 compared with PROBE17 in both the Na and Mg buffer. However, there is an increase in DdT of PROBE17 in comparison to PROBE18 of about 1.5 and 1.0° C. for the Na and Mg hybridization buffers respectively. This difference between PROBE17 and PROBE18 clearly illustrates an advantage of introducing mismatches into LNA-containing probes to enhance probe discrimination.

### EXAMPLE 12

### Effect of 2′-Modification on the Hybridization of Symmetric Tₘ 34-mer Probes

In a further analysis of the benefits of LNA, PROBE19 and PROBE20 use the same symmetric Tₘ probe design as Examples 4 and 10, with a number of LNA nucleotides introduced into the sequence. According to the Exiqon Tₘ model, the 5′ segment of PROBE19 complementary to ABL has a Tₘ of 71° C., while the 3′ segment complementary to BCR has a Tₘ of 72° C. These segments clearly have very similar Tₘs but different lengths.

The advantages of the symmetric Tₘ probe design according to this invention are illustrated by the DdT values listed in the Table 3. PROBE19 has a Tₘ of 80.5° C. for hybridization with B2A2_FRET and a DdT value of 12.5° C. in the Na buffer. PROBE19 has a Tₘ of 88.5° C. for hybridization with B2A2_FRET and a DdT value of 9.5° C. in the Mg buffer. Similarly, PROBE20 has a Tₘ of 72.5° C. for hybridization with B2A2_FRET and a DdT value of 17° C. in the Na buffer. Furthermore, PROBE20 has a Tₘ of 81.5 for hybridization with B2A2_FRET and DdT value of 12° C. in the Mg buffer.

It can be seen by comparison of the DdT values of PROBE19 with PROBE15 and the corresponding DdT values of PROBE20 with PROBE16 that there is a significant increase in DdT values as a result of introducing LNA nucleotides into the probes of this invention. Furthermore, by comparison of the DdT value of PROBE19 with PROBE17 and the corresponding DdT values of PROBE20 with PROBE18 it can be seen that there is a significant increase in DdT values for the symmetric Tₘ probe design according to this invention, although the lengths of these symmetric Tₘ probes is slightly different from the symmetric length 30-mers. Moreover, the introduction of the mismatches into PROBE20 reduces the overall Tₘ of PROBE20 compared with PROBE19 in both the Na and Mg buffer. However, there is an increase in DdT of PROBE20 in comparison to PROBE19 of about 4.5 and 2.5° C. for the Na and Mg buffers respectively.

Finally, it can be seen by comparison of the DdT values of PROBE20 (DdT=17° C. in Na Buffer) with PROBE9 (DdT=3° C. in Na Buffer) that there is a substantial additive benefit from combining all of the probe design features according to this invention, resulting in a large overall increase in probe specificity.

As discussed above, in some aspect of the invention it may be preferable to employ nucleic acid analogues with enhanced binding affinity compared to natural phosphodiester deoxyribosenucleic acids, particularly RNA analogues such as 2′-O-methyl RNA or 2′-Fluoro RNA. It is expected that these analogues will have a similar effect on probe binding as the switch to LNA shown in Examples 11 and 12, i.e. there will be a general increase in the Tₘ of the probe compared to the corresponding DNA probe along with an increase in specificity when used against RNA targets.

Although the present invention has been described with reference to preferred or exemplary embodiments, those skilled in the art will recognize that various modifications and variations to the same can be accomplished without departing from the spirit and scope of the present invention and that such modifications are clearly contemplated herein. No limitation with respect to the specific embodiments disclosed herein and set forth In the appended claims is intended nor should any be inferred.

All documents cited herein are incorporated by reference in their entirety.

What is claimed:

1. An oligonucleotide which is hybridisable with greater affinity to a target nucleic acid than to a nucleic acid variant of the target nucleic acid, the target nucleic acid comprising a first domain and a second domain flanking a domain junction, the first domain having a sequence which is conserved with a first sequence in the nucleic acid variant, wherein the oligonucleotide has a first portion and a second portion flanking a portion junction, the first portion being complementary in part to the first domain and the second portion being complementary in part to the second domain, but wherein the first portion comprises at least a first discontinuity relative to the first domain and the second portion comprises at least a second discontinuity relative to the second domain, each discontinuity comprising or consisting of a sequence mismatch and/or a non-nucleotide spacer.

2. The oligonucleotide according to claim 1, in which the first and second portions are of substantially equal length.

3. The oligonucleotide according to claim 1, in which the first and second portions are structurally bilaterally symmetrical about the portion junction.

4. The oligonucleotide according to claim 1, in which the first and second portions have substantially the same melting temperature (Tₘ).

5. The oligonucleotide according to claim 1, in which each of the first and second discontinuities are positioned adjacent nucleotide 2 to 20 of the oligonucleotide, relative to the portion junction.

6. The oligonucleotide according to claim 1, in which each discontinuity is of a length equivalent to 1 to 5 nucleotides.

7. The oligonucleotide according to claim 1, in which the sequence mismatch comprises a natural nucleotide which is non-complementary to a base at a corresponding position in the target nucleic acid.

8. The oligonucleotide according to claim 1, in which the sequence mismatch comprises an artificial mismatch.

9. The oligonucleotide according to claim 8, in which the artificial mismatch comprises a universal base analogue or an abasic mismatch.

10. The oligonucleotide according to claim 1, in which each discontinuity is a non-nucleotide spacer selected from the group consisting of polyethylene glycol, a phosphoramidite spacer, a C3 phosphoramidite spacer, and an amino acid.

11. The oligonucleotide according to claim 1, in which the oligonucleotide comprises a natural nucleotide or a nucleotide analogue, such as a 2-O-methyl analogue, a bridged nucleic acid monomer, locked nucleic acid [LNA] monomer, a peptide nucleic acid (PNA) monomer, a universal nucleoside, and combinations thereof.

12. The oligonucleotide according to claim 1, in which the oligonucleotide comprises a label selected from the group consisting of a fluorescent tag, a mass tag, biotin, an enzyme, and a nanoparticle.

13. The oligonucleotide according to claim 12, in the form of a molecular beacon.

14. The oligonucleotide according to claim 1, the oligonucleotide having a 5’ to 3′ structure comprising the first portion, the portion junction and the second portion, wherein the first portion has a 5′ to 3′ structure of 1 to 45 nucleotides, a discontinuity, and 2 to 12 nucleotides, and the second portion has a 5′ to 3′ structure of 2 to 12 nucleotides, a discontinuity, and 1 to 45 nucleotides.

15. The oligonucleotide according to claim 1, in which the oligonucleotide is from 1 to 100 nucleotides in length, from 40 to 70 nucleotides in length, less than 40 nucleotides in length, less than 30 nucleotides in length, less than 20 nucleotides in length, or at least 16 nucleotides in length.

16. The oligonucleotide according to claim 1, in which the target nucleic acid is an mRNA molecule.

17. The oligonucleotide according to claim 16, in which the mRNA molecule is a splice variant of a gene, the nucleic acid variant being an alternative splice variant of the same gene.

18. The oligonucleotide according to claim 17, in which the portion junction of the oligonucleotide corresponds in position with the domain junction in the target nucleic acid.

19. The oligonucleotide according to claim 1, in which the target nucleic acid is a chimeric gene or its expressed mRNA.

20. The oligonucleotide according to claim 1, in which the target nucleic acid is a chromosome or any portion thereof.

21. The oligonucleotide according to claim 1, said oligonucleotide capable of use in detection of the target nucleic acid by in situ hybridisation.

22. The oligonucleotide according to claim 1, for use in an array.

23. The oligonucleotide according to claim 1, comprising an antisense oligonucleotide.

24. A set of oligonucleotides comprising two or more oligonucleotides, in which a first oligonucleotide is as defined in claim 1 and a second oligonucleotide is as defined in claim 23 provided that the second or further oligonucleotide is hybridisable with greater affinity to one or more nucleic acid variants of the target nucleic acid.

25. The set of oligonucleotides according to claim 24, for use in simultaneous or sequential detection of the target nucleic acid and the one or more nucleic acid variants.

26. The set of oligonucleotides according to claim 24, wherein said set of oligonucleotides is attached to a solid surface such as an array or a magnetic bead.

27. An array comprising an oligonucleotide as defined by claim 1 or a set of oligonucleotides as defined by claim 24.

28. A method for detecting the presence or absence of a target nucleic acid in a sample, the target nucleic acid comprising a first domain adjacent a second domain, the first domain having a sequence which is conserved with a first sequence in a nucleic acid variant of the, target nucleic acid, comprising:
(i) labelling nucleic acids in the sample;
(ii) contacting the labelled nucleic acids with an oligonucleotide as defined in claim 1 under conditions which allow hybridisation of the oligonucleotide to the target nucleic acid to form a duplex molecule;
(iii) optionally washing the duplex molecule; and
(iv) detecting the presence or absence of the target nucleic target nucleic acid by determining the presence of absence of label bound to the oligonucleotide.

29. A method for detecting the presence or absence of a target nucleic acid and one or more nucleic acid variants of the target nucleic acid in a sample, the target nucleic acid comprising a first domain adjacent a second domain, the first domain having a. sequence which is conserved with a first sequence in the or each nucleic acid variant, comprising the steps of:
(i) labelling nucleic acids in the sample;
(ii) contacting the labelled nucleic acids with a set of oligonucleotides as defined in claim 24 under conditions which allow hybridisation of the oligonucleotides to form duplex molecules with the target nucleic acid and the nucleic acid variants;
(iii) optionally washing the duplex molecules; and
(iv) detecting the presence or absence of the target nucleic acid and the nucleic acid variants by determining the presence of absence of label bound to the oligonucleotides.

30. The method according to claim 28, in which at least one oligonucleotide is immobilised on a solid surface selected from the group consisting of (a matrix, a planar surface, a bead, magnetic bead, or a fluorescently encoded microparticle.

31. A method for detecting the presence or absence of a target nucleic acid in a sample, the target nucleic acid comprising a first domain adjacent a second domain, the first domain having a sequence which is conserved with a first sequence in a nucleic acid variant of the target nucleic acid, comprising:
(i) labelling an oligonucleotide as defined in claim 1 (or providing an oligonucleotide as defined in claim 1 which has been previously labelled;
(ii) contacting the sample with the labelled oligonucleotide under conditions which allow hybridisation of the oligonucleotide to the target nucleic acid to form a duplex molecule;
(iii) optionally washing the duplex molecule; and
(iv) detecting the presence or absence of the target nucleic target nucleic acid by determining the presence of absence of label bound to the oligonucleotide.

32. A method for detecting the presence or absence of a target nucleic acid and one or more nucleic acid variants of the target nucleic acid in a sample, the target nucleic acid comprising a first domain adjacent a second domain, the first domain having a sequence which is conserved with a first sequence in the or each nucleic acid variant, comprising:
(i) labelling a set of oligonucleotides as defined in claim 24 (or providing a set of oligonucleotides as defined in claim 24 which have been previously labelled;
(ii) contacting the sample with the labelled oligonucleotides under conditions which allow hybridisation of the oligonucleotides to form duplex molecules with the target nucleic acid and the nucleic acid variants;
(iii) optionally washing the duplex molecules; and
(iv) detecting the presence or absence of the target nucleic acid and the nucleic acid variants by determining the presence of absence of label bound to the target nucleic acid and the nucleic acid variants.

33. The method according to claim 31, in which the method is an in situ hybridisation method.

34. The method according to claim 33, in which at least one oligonucleotide is labelled with a fluorescent tag, a mass tag, biotin, an enzyme, a nanoparticle and combinations thereof.

35. The method according to claim 28, in which the conditions which allow hybridisation of the oligonucleotide to the target nucleic acid to form a duplex molecule are optimised for salt concentration temperature or both such that the or each oligonucleotide binds with enhanced specificity to its target nucleic acid.

36. The method according to claim 28, further comprising quantifying the amount of target nucleic acid or nucleic acid variant by measuring the amount of bound label.

37. A kit comprising one or more oligonucleotides as defined in claim 1 or a set of oligonucleotides as defined in claim 24.

38. (canceled)


SEQUENCE LISTING (34 sequences)

SEQ ID NO: 1 - protein, 4 aa, Artificial Sequence
  Synthetic peptide
XAPA

SEQ ID NO: 2 - protein, 4 aa, Artificial Sequence
  Synthetic peptide
XAPV

SEQ ID NO: 3 - protein, 4 aa, Artificial Sequence
  Synthetic peptide
XVPA

SEQ ID NO: 4 - protein, 4 aa, Artificial Sequence
  Synthetic peptide
XVPV

SEQ ID NO: 5 - protein, 4 aa, Artificial Sequence
  Synthetic peptide
XADA

SEQ ID NO: 6 - protein, 4 aa, Artificial Sequence
  Synthetic peptide
XADV

SEQ ID NO: 7 - protein, 4 aa, Artificial Sequence
  Synthetic peptide
XVDA

SEQ ID NO: 8 - protein, 4 aa, Artificial Sequence
  Synthetic peptide
XVDV

SEQ ID NO: 9 - DNA, 50 bp, Artificial Sequence
  Synthetic oligonucleotide
attccgctgaccatcaataaggaagaagcccttcagcggccagtagcatc

SEQ ID NO: 10 - DNA, 50 bp, Artificial Sequence
  Synthetic oligonucleotide
cactgcaatgtttttgtggaacatgaagcccttcagcggccagtagcatc

SEQ ID NO: 11 - DNA, 50 bp, Artificial Sequence
  Synthetic oligonucleotide
attccgctgaccatcaataaggaagatgatgagtctccggggctctatgg

SEQ ID NO: 12 - DNA, 40 bp, Artificial Sequence
  Synthetic oligonucleotide
tactggccgctgaagggcttcttccttattgatggtcagc

SEQ ID NO: 13 - DNA, 40 bp, Artificial Sequence
  Synthetic oligonucleotide
tactggccgccgaagggcttcttccttatcgatggtcagc

SEQ ID NO: 14 - DNA, 40 bp, Artificial Sequence
  Synthetic oligonucleotide
ggccgctgaagggcttcttccttattgatggtcagcggaa

SEQ ID NO: 15 - DNA, 40 bp, Artificial Sequence
  Synthetic oligonucleotide
ggccgccgaagggcttcttccttattgattgtcagcggaa

SEQ ID NO: 16 - DNA, 30 bp, Artificial Sequence
  Synthetic oligonucleotide
gccgctgaagggcttcttccttattgatgg

SEQ ID NO: 17 - DNA, 30 bp, Artificial Sequence
  Synthetic oligonucleotide
gccgctgtagggcttcttcctttttgatgg

SEQ ID NO: 18 - DNA, 34 bp, Artificial Sequence
  Synthetic oligonucleotide
ccgctgaagggcttcttccttattgatggtcagc

SEQ ID NO: 19 - DNA, 34 bp, Artificial Sequence
  Synthetic oligonucleotide
ccgctgtagggcttcttccttattgttggtcagc

SEQ ID NO: 20 - DNA, 50 bp, Artificial Sequence; identical to SEQ ID NO: 9
  Synthetic oligonucleotide

SEQ ID NO: 21 - DNA, 50 bp, Artificial Sequence; identical to SEQ ID NO: 10
  Synthetic oligonucleotide

SEQ ID NO: 22 - DNA, 50 bp, Artificial Sequence; identical to SEQ ID NO: 11
  Synthetic oligonucleotide

SEQ ID NO: 23 - DNA, 40 bp, Artificial Sequence; identical to SEQ ID NO: 12
  Synthetic oligonucleotide

SEQ ID NO: 24 - DNA, 40 bp, Artificial Sequence; identical to SEQ ID NO: 13
  Synthetic oligonucleotide

SEQ ID NO: 25 - DNA, 40 bp, Artificial Sequence; identical to SEQ ID NO: 14
  Synthetic oligonucleotide

SEQ ID NO: 26 - DNA, 40 bp, Artificial Sequence; identical to SEQ ID NO: 15
  Synthetic oligonucleotide

SEQ ID NO: 27 - DNA, 30 bp, Artificial Sequence; identical to SEQ ID NO: 16
  Synthetic oligonucleotide

SEQ ID NO: 28 - DNA, 30 bp, Artificial Sequence; identical to SEQ ID NO: 17
  Synthetic oligonucleotide

SEQ ID NO: 29 - DNA, 34 bp, Artificial Sequence; identical to SEQ ID NO: 18
  Synthetic oligonucleotide

SEQ ID NO: 30 - DNA, 34 bp, Artificial Sequence; identical to SEQ ID NO: 19
  Synthetic oligonucleotide

SEQ ID NO: 31 - DNA, 30 bp, Artificial Sequence; identical to SEQ ID NO: 16
  Synthetic oligonucleotide

SEQ ID NO: 32 - DNA, 30 bp, Artificial Sequence; identical to SEQ ID NO: 17
  Synthetic oligonucleotide

SEQ ID NO: 33 - DNA, 34 bp, Artificial Sequence; identical to SEQ ID NO: 18
  Synthetic oligonucleotide

SEQ ID NO: 34 - DNA, 34 bp, Artificial Sequence; identical to SEQ ID NO: 19
  Synthetic oligonucleotide